(19) United States
(12) Patent Application Publication
(10) Pub. No.: US 2025/0025450 A1
(43) Pub. Date: Jan. 23, 2025

(54) METHODS OF TREATING INTERSTITIAL LUNG DISEASES AND OTHER FIBROTIC-MEDIATED PULMONARY DISEASES AND DISORDERS WITH DEUPIRFENIDONE

(71) Applicant: PureTech LYT 100, Inc., Boston, MA (US)

(72) Inventors: Michael C. Chen, Norwood, MA (US); Eric Elenko, Boston, MA (US); Camilla S. Graham, Framingham, MA (US); Varun Garg, Framingham, MA (US)

(21) Appl. No.: 18/899,597

(22) Filed: Sep. 27, 2024

Related U.S. Application Data
(63) Continuation of application No. PCT/US2023/017213, filed on Mar. 31, 2023.
(60) Provisional application No. 63/432,208, filed on Dec. 13, 2022.
(60) Provisional application No. 63/431,530, filed on Dec. 9, 2022.
(60) Provisional application No. 63/403,481, filed on Sep. 2, 2022.
(60) Provisional application No. 63/374,362, filed on Sep. 1, 2022.
(60) Provisional application No. 63/356,653, filed on Jun. 29, 2022.
(60) Provisional application No. 63/352,107, filed on Jun. 14, 2022.
(60) Provisional application No. 63/341,828, filed on May 13, 2022.
(60) Provisional application No. 63/341,269, filed on May 12, 2022.
(60) Provisional application No. 63/341,279, filed on May 12, 2022.
(60) Provisional application No. 63/341,281, filed on May 12, 2022.
(60) Provisional application No. 63/326,132, filed on Mar. 31, 2022.
(60) Provisional application No. 63/326,129, filed on Mar. 31, 2022.

(51) Int. Cl. A61K 31/4412; A61P 11/00

(52) U.S. Cl. CPC A61K 31/4412; A61P 11/00

(57) ABSTRACT

Disclosed herein is a method of treating interstitial lung disease and other fibrotic-mediated pulmonary diseases or disorders. The method includes administering to a subject in need thereof the deuterium-enriched pirfenidone LYT-100 at a total daily dose from about 825 mg to about 2475 mg.

### CROSS-REFERENCE TO RELATED APPLICATIONS

This application is a continuation of International Application No. PCT/US2023/017213, filed Mar. 31, 2023 which claims the benefit of U.S. Provisional Application No. 63/432,208, filed Dec. 13, 2022, and claims the benefit of U.S. Provisional Application No. 63/431,530, filed Dec. 9, 2022, and claims the benefit of U.S. Provisional Application No. 63/403,481, filed Sep. 2, 2022, and claims the benefit of U.S. Provisional Application No. 63/374,362, filed Sep. 1, 2022, and claims the benefit of U.S. Provisional Application No. 63/356,653, filed Jun. 29, 2022, and claims the benefit of U.S. Provisional Application No. 63/352,107, filed Jun. 14, 2022, and claims the benefit of U.S. Provisional Application No. 63/341,828, filed May 13, 2022, and claims the benefit of U.S. Provisional Application No. 63/341,269, filed May 12, 2022, and claims the benefit of U.S. Provisional Application No. 63/341,279, filed May 12, 2022, and claims the benefit of U.S. Provisional Application No. 63/341,281, filed May 12, 2022, and claims the benefit of U.S. Provisional Application No. 63/326,132, filed Mar. 31, 2022, and claims the benefit of U.S. Provisional Application No. 63/326,129, filed Mar. 31, 2022, all of which are herein incorporated by reference in their entirety and for all purposes.

### BACKGROUND

Current antifibrotics suffer from, among other things, poor drug tolerability, including dose-limiting side effects and toxicity associated with gastrointestinal intolerability (e.g., nausea, diarrhea, and other GI events), headache, and photosensitivity, as well as other adverse side effects, which significantly limitcurrent treatments for interstitial lung diseases and other fibrotic-mediated pulmonary diseases and disorders. The dose-limiting side effects and/or toxicity typically require, and are therefore managed by, one or more of the following treatment options: administration of lower, less efficacious doses, periodic reduction(s) of efficacious dose, periodic or permanent cessation of drug (treatment interruption or discontinuation), and/or inability to maintain patients on a sustained treatment program or long-term maintenance dose (e.g., without treatment interruption). For example, pirfenidone, one of only two drugs currently approved in the US by the FDA for treatment of idiopathic pulmonary fibrosis (IPF), suffers from poor tolerability issues which significantly limit the usage of the drug, resulting in dose reduction, switch of drug, and/or interruption or discontinuation of antifibrotic therapy. Studies (see, e.g., Dempsey, 2021) have indicated that only 21% of patients who initiated therapy with pirfenidone remained on pirfenidone at the recommended dose after 2 years. Poor tolerability and the aforementioned management thereof, including a significant incidence of permanent dose reduction and treatment discontinuation, is associated with reduced clinical efficacy and a lost opportunity for full clinical benefit.

Accordingly, there exists a need for a therapy having a superior tolerability profile compared to current antifibrotics for the treatment of interstitial lung disease and other fibrotic-mediated pulmonary diseases and disorders. Particularly, there exists a need for a treatment option that allows for dosing which can achieve higher drug exposure than the current treatment options which are limited due to dose-limiting side effects and/or toxicity, which possess a superior tolerability profile compared to current antifibrotics, or both, such that continuous (e.g., uninterrupted) treatment can be maintained.

### SUMMARY

In one aspect is provided a method of treating an interstitial lung disease or other fibrotic-mediated pulmonarydisease or disorder, the method comprising administering to a subject in need thereof total daily dose from about 825 to about 2475 mg of a deuterium-enriched pirfenidone having the structure:

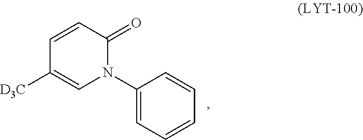 [SMILES: [2H]C([2H])([2H])c1ccc(=O)n(-c2ccccc2)c1]

wherein the interstitial lung disease or other fibrotic-mediated pulmonarydisease or disorder is treated in the subject.

In some embodiments, the total daily dose is 1650 mg.

In some embodiments, the total daily dose is 2475 mg.

In some embodiments, the total daily dose is administered in three equal administrations.

In some embodiments, the total daily dose is administered in three equal administrations of 825 mg each (825 mg TID).

In some embodiments, the total daily dose is administered in three equal administrations of 550 mg each (550 mg TID).

In some embodiments, the LYT-100 is administered without regard to food.

In some embodiments, the LYT-100 is administered without food.

In some embodiments, the LYT-100 is administered with food.

In some embodiments, the LYT-100 is administered without dose escalation.

In some embodiments, administering comprises titrating up to the total daily dose from an initial total daily dose which is below the total daily dose.

In some embodiments, administering comprises titrating up to the total daily dose from an initial total daily dose which is below the total daily dose, and wherein titrating comprises administering the LYT-100 in three daily doses of 550 mg each for an initial period of time, followed by administering the LYT-100 in three daily doses of 825 mg each for a period of time. In some embodiments, the titrating comprises administering LYT-100 in three daily doses of 275 mg each for an initial period of time, followed by administering the the LYT-100 in three daily doses of 550 mg each for a period of time, optionally followed by administering the LYT-100 in three daily doses of 825 mg each for a period of time. In some embodiments, the initial period of time is 3-14 days. In some embodiments, the initial period of time is 3-7 days.

In some embodiments, the fibrotic-mediated pulmonary disease or disorder is an interstitial lung disease (ILD). In some embodiments, the ILD is an exposure-related ILD, a drug-induced ILD, an autoimmune interstitial lung disease, unclassifiable interstitial lung disease (uILD), progressive fibrotic interstitial lung disease (pfILD), respiratory bronchiolitis-ILD (RB-ILD), a connective tissue disease-related ILD (CTD-ILD), rheumatoid arthritis (RA-ILD), systemic sclerosis (SSc-ILD), mixed connective tissue disease-ILD, scleroderma related ILD, or ILD related to chronic sarcoidosis. In some embodiments, the ILD is a progressive fibrosing ILD (PF-ILD).

In some embodiments, the interstitial lung disease disease or disorder is not idiopathic pulmonary fibrosis (IPF).

In some embodiments, the interstitial lung disease or other fibrotic-mediated pulmonary disease or disorder is alleviated.

In some embodiments, progression of the interstitial lung disease or other fibrotic-mediated pulmonary disease or disorder is delayed, slowed, or arrested.

In another aspect is provided a method of treating an interstitial lung disease or other fibrotic-mediated pulmonary disease or disorder, the method comprising administering to a subject in need thereof a deuterium-enriched pirfenidone having the structure:

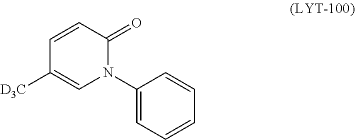 [SMILES: [2H]C([2H])([2H])c1ccc(=O)n(-c2ccccc2)c1]

wherein the administering is at a dose that achieves a systemic exposure of LYT-100 in the subject which is the same or about the same as the systemic exposure of pirfenidone achieved when pirfenidone is administered at a total daily dose of 2403 mg.

In some embodiments, the dose of LYT-100 is a total daily dose of 1650 mg.

In some embodiments, the total daily dose is administered in three equal administrations.

In some embodiments, the total daily dose is administered in three equal administrations of 550 mg each (550 mg TID).

In some embodiments, the LYT-100 is administered without regard to food.

In some embodiments, the LYT-100 is administered without food.

In some embodiments, the LYT-100 is administered with food.

In some embodiments, the LYT-100 is administered without dose escalation.

In some embodiments, the fibrotic-mediated pulmonary disease or disorder is an interstitial lung disease (ILD). In some embodiments, the ILD is an exposure-related ILD, a drug-induced ILD, an autoimmune interstitial lung disease, unclassifiable interstitial lung disease (uILD), progressive fibrotic interstitial lung disease (pfILD), respiratory bronchiolitis-ILD (RB-ILD), a connective tissue disease-related ILD (CTD-ILD), rheumatoid arthritis (RA-ILD), systemic sclerosis (SSc-ILD), mixed connective tissue disease-ILD, scleroderma related ILD, or ILD related to chronic sarcoidosis. In some embodiments, the ILD is a progressive fibrosing ILD (PF-ILD).

In some embodiments, the interstitial lung disease disease or disorder is not idiopathic pulmonary fibrosis (IPF). In some embodiments, the interstitial lung disease or other fibrotic-mediated pulmonaryd isease or disorder is alleviated.

In some embodiments, progression of the interstitial lung disease or other fibrotic-mediated pulmonary disease or disorder is delayed, slowed, or arrested.

In yet another aspect is provided a method of treating a interstitial lung disease or other fibrotic-mediated pulmonary disease or disorder, the method comprising administering to a subject in need thereof a deuterium-enriched pirfenidone having the structure:

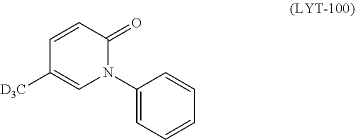 [SMILES: [2H]C([2H])([2H])c1ccc(=O)n(-c2ccccc2)c1]

wherein the administering is at a dose that achieves a systemic exposure of LYT-100 in the subject which is greater than the systemic exposure of pirfenidone achieved when pirfenidone is administered at a total daily dose of 2403 mg.

In some embodiments, the administering is at a dose that achieves a systemic exposure of LYT-100 in the subject which is greater than the systemic exposure of pirfenidone achieved when pirfenidone is administered at a total daily dose of 2403 mg, wherein the total daily dose of pirfenidone is administered in three doses of 801 mg each (801 mg TID).

In some embodiments, the administering is at a dose that achieves a systemic exposure of LYT-100 in the subject which is about 1.1× to about 1.9× the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is about 1.25× to about 1.75× the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the dose of LYT-100 administered achieves a systemic exposure that is 1.25× to 1.75× the systemic exposure of pirfenidone achieved when pirfenidone is administered at a total daily dose of 2403 mg.

In some embodiments, the administering is at a dose that achieves a systemic exposure of LYT-100 in the subject which is about 1.4× to 1.6× the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is 1.4× to 1.5× the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is about 1.5× the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is about 85% to about 125% the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is 125% to 175% the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is 140% to 160% greater than the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is 140% to 150% greater than the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is about 150% of the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is about 10% to about 90% greater than the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is about 25% to about 75% greater than the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is 25% to 75% greater than the systemic exposure of pirfenidone achieved when pirfenidone is administered at a total daily dose of 2403 mg, optionally wherein the total daily dose of pirfenidone is administered in three doses of 801 mg each (801 mg TID). In some embodiments, the administering is at a dose that achieves a systemic exposure of LYT-100 in the subject which is 40% to 60% greater than the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is 40% to 50% greater than the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the administering is at a dose that achieves a systemic exposure of LYT-100 in the subject which is about 50% greater than the systemic exposure of pirfenidone achieved when pirfenidone is administered at a total daily dose of 2403 mg, optionally wherein the total daily dose of pirfenidone is administered in three doses of 801 mg each (801 mg TID).

In some embodiments, the dose of LYT-100 that achieves a systemic exposure of LYT-100 in the subject which is greater than the systemic exposure of pirfenidone achieved when pirfenidone is administered at a total daily dose of 2403 mg is a total daily dose of 2475 mg.

In some embodiments, the total daily dose is administered in three equal administrations.

In some embodiments, the total daily dose is administered in three equal administrations of 825 mg each (825 mg TID).

In some embodiments, the LYT-100 is administered without regard to food.

In some embodiments, the LYT-100 is administered without food.

In some embodiments, the LYT-100 is administered with food.

In some embodiments, the LYT-100 is administered without dose escalation.

In some embodiments, administering comprises titrating up to the total daily dose from an initial total daily dose which is below the total daily dose.

In some embodiments, administering comprises titrating up to the total daily dose from an initial total daily dose which is below the total daily dose, and wherein titrating comprises administering the LYT-100 in three daily doses of 550 mg each for an initial period of time, followed by administering the LYT-100 in three daily doses of 825 mg each for a period of time. In some embodiments, the titrating comprises administering LYT-100 in three daily doses of 275 mg each for an initial period of time, followed by administering the the LYT-100 in three daily doses of 550 mg each for a period of time, optionally followed by administering the LYT-100 in three daily doses of 825 mg each for a period of time. In some embodiments, the initial period of time is 3-14 days. In some embodiments, the initial period of time is 3-7 days.

In some embodiments, the fibrotic- or collagen-mediated disease or disorder is an interstitial lung disease (ILD). In some embodiments, the ILD is an exposure-related ILD, a drug-induced ILD, an autoimmune interstitial lung disease, unclassifiable interstitial lung disease (uILD), progressive fibrotic interstitial lung disease (pfILD), respiratory bronchiolitis-ILD (RB-ILD), a connective tissue disease-related ILD (CTD-ILD), rheumatoid arthritis (RA-ILD), systemic sclerosis (SSc-ILD), mixed connective tissue disease-ILD, scleroderma related ILD, or ILD related to chronic sarcoidosis. In some embodiments, the ILD is a progressive fibrosing ILD (PF-ILD).

In some embodiments, the fibrotic- or collagen-mediated disease or disorder is not idiopathic pulmonary fibrosis (IPF).

In some embodiments, the fibrotic- or collagen-mediated disease or disorder is alleviated.

In some embodiments, progression of the fibrotic- or collagen-mediated disease or disorder is delayed, slowed, or arrested.

### BRIEF DESCRIPTION OF THE DRAWINGS

FIG. 1 is a graphical illustration of a crossover clinical trial study design according to a non-limiting embodiment of the disclosure.

FIG. 2 is a graphical illustration of another crossover clinical trial study design according to a non-limiting embodiment of the disclosure.

FIG. 3 is a table showing the extrapolated steady-state exposures (AUC₂₄ₛₛ) and steady-state Cₘₐₓ values of LYT-100 for 450 mg-550 mg TID dosing based on PK data from two separate cohorts (12A and 12B) and a pooled dataset. The pharmacokinetic parameters were calculated using steady state AUC₀₋₂₄ after administration of LYT-100 dosed at 1000 mg BID or pifenidone dosed at 801 mg TID. The data demonstrates that a dose of 550 mg TID LYT-100 has a steady-state exposure (AUC) that is calculated to be equivalent to 98.5% of the steady-state exposure (AUC) of pirfenidone dosed at 801 mg TID, and a Cₘₐₓ that is calculated to be equivalent to 67.4% of the Cₘₐₓ of pirfenidone dosed at 801 mg TID.

FIG. 4 is a table showing the extrapolated steady-state exposures (AUC₂₄ₛₛ) and steady-state Cₘₐₓ values of LYT-100 for 700 mg-1000 mg BID dosing (1400 mg-2000 mg daily dose) versus 450 mg-850 mg TID dosing (1350 mg-2550 mg daily dose). The data demonstrates that a dose of 825 mg BID LYT-100 (1650 mg daily dose) has a steady-state exposure (AUC) that is calculated to be equivalent to 98.5% of the steady-state exposure (AUC) and 101.1% of the steady-state Cₘₐₓ of pirfenidone dosed at 801 mg TID. In contrast, a dose of 550 mg TID LYT-100 (1650 mg daily dose) has a steady-state exposure (AUC) that is calculated to be equivalent to 98.5% of the steady-state exposure (AUC) and 67.4% of the steady-state Cₘₐₓ of pirfenidone dosed at 801 mg TID.

FIG. 5A is a summary of the pharmacokinetic and tolerability results of a Phase 1 cross-over study conducted in healthy adults dosed with 850 mg BID LYT-100.

FIG. 5B is a table showing the incidence of treatment-emergent adverse events (TEAEs) in a cross-over study of healthy older adults comparing LYT-100 850 mg BID versus pirfenidone 801 mg TID. The data shows that the incidence of gastrointestinal AEs with LYT-100 was 37.1% with LYT-100 versus 29.7% with pirfenidone; the incidence of nervous system AEs was 45.7% with LYT-100 versus 35.1% with pirfenidone; and the incidence of nausea was increased with both LYT-100 and pirfenidone when dosed after fasting.

FIG. 6 is a graphical depiction of side effects encountered in a healthy older patient population for LYT-100 at 550 mg TID and pirfenidone at 801 mg TID.

FIG. 7A is a graphical depiction of time versus exposure for LYT-100 for a dose of 550 mg TID.

FIG. 7B is a graphical depiction of time versus exposure for LYT-100 for a dose of 824 mg TID.

FIG. 7C is a graphical depiction of time versus exposure for the major metabolite for a dose of 550 mg TID.

FIG. 7D is a graphical depiction of time versus exposure for the major metabolite for a dose of 824 mg TID.

FIG. 8 is a table showing the pharmacokinetic parameters for LYT-100 and the major metabolite for doses of 550 mg TID and 824 mg TID.

FIG. 9A is a graphical depiction of time versus exposure for LYT-100 for doses of 550 mg TID and 824 mg TID in the crossover study of Example 1 and two prior dosing studies.

FIG. 9B is a graphical depiction of time versus exposure for the major metabolite for doses of 550 mg TID and 824 mg TID in the crossover study of Example 1 and two prior dosing studies.

FIG. 10 is a graphical illustration of the mean plasma concentrations over time for pirfenidone dosed at 801 mg TID, and for LYT-100 dosed at 550 mg TID and 824 mg TID.

FIG. 11 is a graphical illustration of the mean plasma concentrations of the major metabolite over time for pirfenidone dosed at 801 mg TID, and for LYT-100 dosed at 550 mg TID and 824 mg TID.

FIG. 12 is a graphical depiction of plasma concentration versus time for pirfenidone at 550 mg TID and LYT-100 at 824 mg TID following day 3 in the crossover study of Example 1.

FIG. 13A is a graphical depiction of subject weight versus exposure for LYT-100 for 550 mg TID and 824 mg TID doses in the crossover study of Example 1 and in three prior dosing studies.

FIG. 13B is a graphical depiction of subject weight versus exposure for the major metabolite for 550 mg TID and 824 mg TID doses in the crossover study of Example 1 and in three prior dosing studies.

FIG. 14A is a graphical depiction of subject age versus exposure for LYT-100 normalized to 550 mg TID in the crossover study of Example 1 and in three prior dosing studies.

FIG. 14B is a graphical depiction of subject age versus exposure for the major metabolite of LYT-100 normalized to 550 mg TID in the crossover study of Example 1 and in three prior dosing studies.

FIG. 15A is a graphical summary of exposure versus dose in the crossover study of Example 1 and a prior dosing study demonstrating the achievement of bioequivalence to 801 mg TID pirfenidone for 550 mg TID LYT-100.

FIG. 15B is a graphical summary of exposure versus dose in the crossover study of Example 1 and a prior dosing study demonstrating the achievement of bioequivalence to 801 mg TID pirfenidone for 550 mg TID LYT-100.

FIG. 15C is a graphical summary of exposure versus dose in the crossover study of Example 1 and a prior dosing study demonstrating the achievement of bioequivalence to 801 mg TID pirfenidone for 550 mg TID LYT-100.

FIG. 15D is a graphical summary of exposure versus dose in the crossover study of Example 1 and pooled data from a prior dosing study demonstrating the achievement of bioequivalence to 801 mg TID pirfenidone for 550 mg TID LYT-100.

FIG. 16 is a graphical summary of exposure versus dose for pooled data from the crossover study of Example 1 and three prior dosing studies and demonstrating the achievement of bioequivalence to 801 mg TID pirfenidone for 550 mg TID and 687 mg TID LYT-100.

FIG. 17 is a table showing the predicted bioequivalence for various LYT-100 TID doses using data from the crossover study of Example 1 and three prior dosing studies.

FIG. 18A is a graphical cartoon illustration of predicted plasma concentrations over time for pirfenidone at 801 mg TID, LYT-100 at 550 mg TID, and LYT-100 at 825 mg TID.

FIG. 18B is a table showing the ratio of predicted plasma concentrations for pirfenidone at 801 mg TID versus LYT-100 dosed at 550 mg TID and 825 mg TID.

FIG. 19 is a table showing a summary of baseline demographic characteristics with respect to age and sex for subjects in the COVID-19 clinical study of Example 3.

FIG. 20 is a table showing a summary of baseline demographic characteristics with respect to ethnicity, race, and time from COVID diagnosis for subjects in the COVID-19 clinical study of Example 3.

FIG. 21 is a table showing a summary of subject disposition for the enrolled population in the COVID-19 clinical study of Example 3.

FIG. 22 is a table showing a summary of treatment emergent adverse events judged to be at least possibly related to LYT-100 in the COVID-19 clinical study of Example 3.

FIG. 23 is a table showing the metabolism of pirfenidone and LYT-100 in the presence of individual CYP isozymes in the assay of Example 4.

FIG. 24 is a graphical depiction of activity results for LYT-100 and pirfenidone in the BioMap Fibrosis Panel of Example 5.

FIG. 25A is a graphical illustration showing that TNF-α response to LPS was reduced by pretreatment with both pirfenidone and LYT-100.

FIG. 25B is a graphical illustration showing that IL-6 response to LPS was reduced by pretreatment with both pirfenidone and LYT-100.

FIG. 26 depicts representative photomicrographs of Sirius-red stained liver sections demonstrating that LYT-100 significantly reduced the area of fibrosis.

FIG. 27 is a graphical illustration showing the percent fibrosis area for LYT-100 versus vehicle and control.

FIG. 28A is a graphical illustration showing that LYT-100 does not induce survival of Primary Mouse Lung Fibroblasts (PMFL).

FIG. 28B and FIG. 28C are graphical illustrations showing that LYT-100 reduced TGF-β-induced total collagen level in PMFLs in a 6-well and 96-well format, respectively.

FIG. 28D and FIG. 28E are graphical illustrations showing that LYT-100 reduced TGF-β-induced soluble fibronectin levels and soluble collagen levels.

FIG. 29A is a graphical illustration showing that LYT-100 does not affect survival of L929 cells.

FIG. 29B is a graphical illustration showing that LYT-100 inhibits TGF-induced collagen synthesis.

FIG. 29C is a graphical illustration showing that LYT-100 significantly inhibits TGF-β-induced total collagen levels.

FIG. 29D is a graphical illustration showing that LYT-100 significantly inhibits TGF-β-induced soluble collagen levels.

FIG. 29E is a graphical illustration showing that LYT-100 signficantly reduces soluble fibronectin levels in the absence and presence of TGF-β-induction.

FIGS. 30A-30D depict results of once daily administration of LYT-100 to reduce swelling in a mouse lymphedema model.

FIG. 31 is a graphical depiction of percent change in body weight over time for rats in Phase I of the bleomycin induced lung fibrosis model of Example 11.

FIG. 32A is a graphical depiction of lung weight to body weight percentage over time for rats in Phase I of the bleomycin induced lung fibrosis model of Example 11.

FIG. 32B is a graphical depiction of lung weight to body weight percentage over time for rats in Phase I of the bleomycin induced lung fibrosis model of Example 11.

FIG. 33A is a graphical depiction of body weight over time for rats in Phase II of the bleomycin induced lung fibrosis model of Example 11.

FIG. 33B is a graphical depiction of percent change in body weight over time for rats in Phase II of the bleomycin induced lung fibrosis model of Example 11.

FIG. 34A is a graphical depiction of lung weight over time for rats in Phase II of the bleomycin induced lung fibrosis model of Example 11.

FIG. 34B is a graphical depiction of lung weight over time for rats in Phase II of the bleomycin induced lung fibrosis model of Example 11.

FIG. 35A is a graphical depiction of lung weight to body weight percentage over time for rats in Phase II of the bleomycin induced lung fibrosis model of Example 11.

FIG. 35B is a graphical depiction of lung weight to body weight percentage over time for rats in Phase II of the bleomycin induced lung fibrosis model of Example 11.

FIG. 36A is a graphical depiction of hydroxyproline content in left lung tissue for rats in Phase II of the bleomycin induced lung fibrosis model of Example 11.

FIG. 36B is a graphical depiction of hydroxyproline content in left lung tissue for rats in Phase II of the bleomycin induced lung fibrosis model of Example 11.

FIG. 37 is a table showing the hydroxyproline content in left lung tissue across the various treatment groups in Phase II of the bleomycin induced lung fibrosis model of Example 11.

FIG. 38A is a graphical depiction of hydroxyproline content in lung tissue across the various treatment groups in Phase II of the bleomycin induced lung fibrosis model of Example 11.

FIG. 38B is a graphical depiction of hydroxyproline content in lung tissue across the various treatment groups in Phase II of the bleomycin induced lung fibrosis model of Example 11.

FIG. 39 is a table showing the hydroxyproline content in lung tissue across the various treatment groups in Phase II of the bleomycin induced lung fibrosis model of Example 11.

FIG. 40A is a graphical depiction of mean lung fibrosis score across the various treatment groups in Phase II of the bleomycin induced lung fibrosis model of Example 11.

FIG. 40B is a graphical depiction of mean lung fibrosis score across the various treatment groups in Phase II of the bleomycin induced lung fibrosis model of Example 11.

FIG. 40C is a graphical depiction of median lung fibrosis score across the various treatment groups in Phase II of the bleomycin induced lung fibrosis model of Example 11.

FIG. 40D is a graphical depiction of median lung fibrosis score across the various treatment groups in Phase II of the bleomycin induced lung fibrosis model of Example 11.

FIG. 41 is a graphical depiction of frequency of lung fibrosis scores across the various treatment groups in Phase II of the bleomycin induced lung fibrosis model of Example 11.

### DETAILED DESCRIPTION

Disclosed herein is a method of treating an interstitial lung disease or other fibrotic-mediated pulmonary disease or disorders, the method comprising administering to a subject in need thereof LYT-100. In some embodiments, the method comprises administering a total daily dose of LYT-100 that achieves a systemic exposure comparable to (e.g., the same or about the same as) the systemic exposure of 2403 mg daily dosing of pirfenidone (including, e.g., 801 mg TID dosing). In some embodiments, the method comprises administering a total daily dose of LYT-100 that achieves a systemic exposure greater than the systemic exposure of pirfenidone dosed at 2403 mg daily dose, e.g., 801 mg TID dosing. The method may in some embodiments engender increased patient compliance, provide a higher exposure of LYT-100 than that of pirfenidone achieved with the currently approved dose (801 mg TID) of pirfenidone, or both, and can ultimately result in a more effective therapeutic agent to address the underlying mechanisms of fibrotic- or collagen-mediated diseases and disorders.

Overall, the results disclosed herein indicate that LYT-100 has the potential for use in treating indications where pirfenidone is shown to have benefit but where tolerability concerns limit its dose, and potentially its efficacy. Accordingly, the disclosed method may be beneficial in treating a range of interstitial lung diseases and other fibrotic-mediated pulmonary diseases and disorders. The features, benefits, and utility of the method are each described further herein below.

### Definitions

While the terms used herein are believed to be well understood by one of ordinary skill in the art, definitions are set forth herein to facilitate explanation of the presently disclosed subject matter.

The articles “a” and “an” are used herein to refer to one or to more than one (i.e., to at least one) of the grammatical object of the article.

The term “about” used throughout this specification is used to describe and account for small fluctuations. For example, the term “about” can refer to greater than, less than or equal to ±10%, such as greater than, less than or equal to ±5%, greater than, less than or equal to ±2%, greater than, less than or equal to ±1%, greater than, less than or equal to ±0.5%, greater than, less than or equal to ±0.2%, greater than, less than or equal to ±0.1% or greater than, ess than or equal to ±0.05%. All numeric values herein are modified by the term “about,” whether or not explicitly indicated. A value modified by the term “about” of course includes the specific value. For instance, “about 5.0” must include 5.0.

The term “Adverse Event” refers to any event, side-effect, or other untoward medical occurrence that occurs in conjunction with the use of a medicinal product in humans, whether or not considered to have a causal relationship to this treatment. An AE can, therefore, be any unfavourable and unintended sign (that could include a clinically significant abnormal laboratory finding), symptom, or disease temporally associated with the use of a medicinal product, whether or not considered related to the medicinal product. Events meeting the definition of an AE include: Exacerbation of a chronic or intermittent pre-existing condition including either an increase in frequency and/or intensity of the condition; New conditions detected or diagnosed after study drug administration that occur during the reporting periods, even though it may have been present prior to the start of the study; Signs, symptoms, or the clinical sequelae of a suspected interaction; Signs, symptoms, or the clinical sequelae of a suspected overdose of either study drug or concomitant medications (overdose per se will not be reported as an AE/SAE). AE's may have a causal relationship with the treatment, may be possibly related, or may be unrelated. Severity of AEs may be graded as one of: Mild (Grade 1): A type of AE that is usually transient and may require only minimal treatment or therapeutic intervention. The event does not generally interfere with usual activities of daily living; Moderate (Grade 2): A type of AE that is usually alleviated with additional specific therapeutic intervention. The event interferes with usual activities of daily living, causing discomfort but poses no significant or permanent risk of harm to the research participant; Severe (Grade 3): A type of AE that interrupts usual activities of daily living, or significantly affects clinical status, or may require intensive therapeutic intervention; Life-threatening (Grade 4): A type of AE that places the participant at immediate risk of death; Death (Grade 5): Events that result in death.

As used herein, the term “clinically effective amount,” “clinically proven effective amount,” and the like, refer to an effective amount of an API as shown through a clinical trial, e.g., a U.S. Food and Drug Administration (FDA) clinical trial.

The term “is/are deuterium,” when used to describe a given variable position in a molecule or formula, or the symbol “D,” when used to represent a given position in a drawing of a molecular structure, means that the specified position is enriched with deuterium above the naturally occurring distribution of deuterium. In some embodiments, deuterium enrichment is of no less than about 1%, no less than about 5%, no less than about 10%, no less than about 20%, no less than about 50%, no less than about 70%, no less than about 80%, no less than about 90%, no less than about 98%, or in some embodiments no less than about 99% of deuterium at the specified position. In some embodiments, the deuterium enrichment is above 90% at each specified position. In some embodiments, the deuterium enrichment is above 95% at each specified position. In some embodiments, the deuterium enrichment is about 99% at each specified position.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods, such as mass spectrometry and nuclear magnetic resonance spectroscopy.

The term “fibrosis” refers to the deposition of extracellular matrix components, excessive fibrous connective tissue, or scarring within an organ or tissue.

The term “idiopathic pulmonary fibrosis (IPF)” refers to a type of lung disease that results in scarring of the lungs (pulmonary fibrosis) for which the origin of the disease state may be unknown.

Terms such as “treating” or “treatment” or “to treat” or “alleviating” or “to alleviate” refer to therapeutic measures that cure, slow down, ameliorate or lessen one or more symptoms of, halt progression of, and/or ameliorate or lessen a diagnosed pathologic condition or disorder. Thus, those in need of treatment include those already with the disorder. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. In some embodiments, a subject is successfully “treated” for a disease or disorder according to the methods provided herein if the patient shows, e.g., total, partial, or transient alleviation or elimination of symptoms associated with the disease or disorder. “treating” can include, but is not limited to, decreasing or alleviating one or more symptoms of the disease or disorder; delaying, slow downing, halting, ameliorating, lessening, and/or decreasing fibrosis; delaying, slow downing, halting, ameliorating or lessening the progression of the disease or disorder; delaying, slow downing, halting, ameliorating or lessening the onset of the disease or disorder; decreasing swelling, inflammation, fibrosis and/or pain; and/or improving pulmonary or respiratory function.

Used in comparison with LYT-100, the term “pirfenidone administered at a dose or dosing that achieves the same or about the same systemic exposure” refers to the dose, dosing, or administration of pirfenidone at which the AUC of pirfenidone in a subject is the same or about the same as the AUC achieved with LYT-100 in a subject at the specified dosing of LYT-100. In some instances, “pirfenidone administered at a dose or dosing that achieves the same or about the same systemic exposure” may refer to pirfenidone administered to a subject at a total daily dose of 2403 mg. In some instances, “pirfenidone administered at a dose or dosing that achieves the same or about the same systemic exposure” may refer to pirfenidone administered to a subject at 801 mg TID.

The term “pharmaceutical composition” refers to a preparation that is in such form as to permit the biological activity of the active ingredient to be effective, and which contains no additional components that are unacceptably toxic to a subject to which the composition would be administered. Pharmaceutical compositions can be in numerous dosage forms, for example, tablet, capsule, liquid, solution, soft gel, suspension, emulsion, syrup, elixir, tincture, film, powder, hydrogel, ointment, paste, cream, lotion, gel, mousse, foam, lacquer, spray, aerosol, inhaler, nebulizer, ophthalmic drops, patch, suppository, and/or enema. Pharmaceutical compositions typically comprise a pharmaceutically acceptable carrier, and can comprise one or more of a buffer (e.g. acetate, phosphate or citrate buffer), a surfactant (e.g. polysorbate), a stabilizing agent (e.g. human albumin), a preservative (e.g. benzyl alcohol), a penetration enhancer, an absorption promoter to enhance bioavailability and/or other conventional solubilizing or dispersing agents. Choice of dosage form and excipients depends upon the active agent to be delivered and the disease or disorder to be treated or prevented, and is routine to one of ordinary skill in the art.

The terms “subject” and “patient” refers to a mammalian subject, including a human subject. In some embodiments, the patient is human subject.

The term “LYT-100” refers to a selectively deuterium-enriched form of pirfenidone. Specifically, LYT-100 is 5-(methyl-d₃)-1-phenylpyridin-2-(1H)-one (CAS #1093951-85-9) which may alternatively be referred to as deupirfenidone or 2(1H)-Pyridinone, 5-(methyl-d3)-1-phenyl. LYT-100 has the following structure:

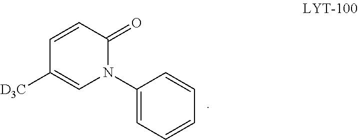 [SMILES: [2H]C([2H])([2H])c1ccc(=O)n(-c2ccccc2)c1]

Reference to “LYT-100” herein further includes any hydrate, solvate, crystalline polymorph, amorphous form, or the like, of 5-(methyl-d₃)-1-phenylpyridin-2-(1H)-one. LYT-100 can be prepared by methods known to one of skill in the art and routine modifications thereof, and/or procedures found in Esaki et al., Tetrahedron 2006, 62, 10954-10961, Smith et al., Organic Syntheses 2002, 78, 51-56, U.S. Pat. Nos. 3,974,281, 8,680,123, WO2003/014087, WO 2008/157786, WO 2009/035598, WO 2012/122165, or WO 2015/112701; the entirety of each of which is hereby incorporated by reference; and references cited therein and routine modifications thereof.

### Methods for Treating Interstitial Lung Disease and Other Fibrotic-Mediated Pulmonary

### Diseases and Disorders

### Pirfenidone

Pirfenidone (Deskar®), CAS #53179-13-8, Pirespa, AMR-69, Pirfenidona, Pirfenidonum, Esbriet, Pirfenex, 5-methyl-1-phenyl-1H-pyridin-2-one, 5-Methyl-1-phenyl-2-(1H)-pyridone, 5-methyl-1-phenylpyridin-2(1H)-one, is an orally administered small molecule with anti-fibrotic effects which has been approved in the United States and elsewhere for treatment of idiopathic pulmonary fibrosis (IPF).

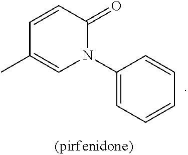

Pirfenidone has established anti-inflammatory and antifibrotic properties. It is likely that multiple mechanisms contribute to the unique profile of pirfenidone. Pirfenidone attenuates fibroblast proliferation, production of fibrosis-associated proteins and cytokines, and biosynthesis and accumulation of extracellular matrix in response to cytokine growth factors such as TGF-β and platelet-derived growth factor, or PDGF (Schaefer et al., Eur Respir Rev. 2011; 20:85-97; InterMune UK, Ltd. Esbriet® Summary of Product Characteristics, 2011). Specifically, pirfenidone blocks the production and activity of TGF-β, a key growth factor that increases collagen production while decreasing its degradation. Moreover, administration of pirfenidone reduces the production of other fibrogenic factors that are induced by TGF-β, such as fibronectin and connective tissue growth factor (Schaefer et al., 2011). Pirfenidone is capable of blocking bleomycin-induced PDGF production as well as fibroblast and hepatic stellate cell proliferation in response to PDGF (DiSario et al., J. Hepatol. 2002 Nov. 37.5.584-591). Pirfenidone inhibits the expression of TNF-α, IL-6, IL-1, and intercellular adhesion molecule 1 (ICAM-1) (Schaefer et al., 2011). In a murine macrophage-like cell line, pirfenidone suppressed TNF-α production or secretion through mitogen-activated protein kinase and c-Jun N-terminal kinase-independent mechanisms and increased the levels of IL-10, an anti-inflammatory cytokine (Schaefer et al., 2011).

In IPF clinical trials, many of the most common adverse reactions were GI (nausea, abdominal pain, diarrhea, dyspepsia, vomiting, and gastroesophageal reflux disease), in addition to fatigue, rash, and photosensitivity reactions. The frequency of these adverse reactions led to discontinuation of 14.6% of patients participating in those clinical trials. In a prospective, real-world observational study of 1009 patients with IPF initiating pirfenidone, 35% of patients had an adverse reaction and dose adjustment, 28% discontinued, and 11% had dose adjustments and discontinued. (Cottin, E R J Open Res, 2018, 4(4)). Consequently, the overall adoption of anti-fibrotic medications, including pirfenidone, has been low. A study used the US OptumLabs Data Warehouse to identify 10,996 patients with IPF with medical and pharmacy claims between Oct. 1, 2014, to Jul. 31, 2019, The study showed that 73.6% of patients with IPF never received an antifibrotic (pirfenidone or nintedanib) during the observation period (Dempsey et al. Ann Am Thorac Soc. 2021; 18(7):1121-1128). In a large post-marketing analysis of 10996 patients diagnosed with IPF, only 13.2% received treatment with pirfenidone during a 5-year follow-up period, the same percentage that received treatment with the other marketed antifibrotic drug nintedanib. (Dempsey, 2021). AEs were noted as a barrier to both adoption and persistence of pirfenidone and nintedanib in IPF patients.

Nintedanib (Ofev; Boehringer Ingelheim) received FDA approval in 2014 for the treatment of patients with idiopathic pulmonary fibrosis. Subsequently, it has been approved for slowing the progression of lung fibrosis in patients with systemic sclerosis (scleroderma), as well as those with other rheumatologic disease who have progressive lung fibrosis (progressive fibrosing interstitial lung disease). Nintedanib is a small molecule that inhibits multiple receptor tyrosine kinases and nonreceptor tyrosine kinases. Specifically, nintedanib inhibits platelet-derived growth factor (PDGF) receptor-alpha and -beta, fibroblast growth factor (FGF) receptor 1-3, vascular endothelial growth factor (VEGF) receptor 1-3, and fms-like tyrosine kinase-3. Of these tyrosine kinase receptors, FGF, PDGF, and VEGF have been implicated in the pathogenesis of idiopathic pulmonary fibrosis. Nintedanib binds competitively to the adenosine triphosphate binding pocket of these receptors and blocks the intracellular signaling, which is crucial for the proliferation, migration, and transformation of fibroblasts, representing essential mechanisms of the idiopathic pulmonary fibrosis pathology.

As reported in Prescribing Information for Ofev, in the study leading to FDA approval, nintedanib was associated with numerous side effects. The most common adverse reactions (≥5%) with nintedanib therapy included diarrhea (62%), nausea (24%), abdominal pain (15%), vomiting (12%), liver enzyme elevation (14%), decreased appetite (11%), headache (8%), weight loss (10%), and hypertension (5%). Overall, 21% of patients who received nintedanib and 15% of patients who received placebo discontinued treatment because of an adverse event. The most frequent adverse reactions leading to the discontinuation of nintedanib were diarrhea, nausea, and decreased appetite. In a 2019 retrospective study, nausea, vomiting or thrombocytopenia was reported to have led to permanent discontinuation of nintedanib, and temporary discontinuation due to adverse effects was common (Nakamura et al. Ann Transl Med. 2019 June; 7(12): 262). Nintedanib may have adverse effects on the liver, and blood tests for liver enzymes are recommended at the start of medication and regular intervals during the first 3 months of treatment.

Pirfenidone has not been tested for clinical efficacy above doses of 801 mg TID due to poor tolerability, including gastrointestinal adverse effects, nausea, weight loss, and photosensitive skin rash (among other AEs). Although some studies have been performed using higher doses of pirfenidone, well-controlled efficacy studies have not yet been done with pirfenidone doses higher than 2403 mg daily dose. Thus, while high doses of pirfenidone—up to 801 mg TID pirfenidone—are associated with improved efficacy in IPF (compared with doses less than 2403 mg daily), an upper threshold to improved clinical efficacy has not been achieved to date because doses higher than 801 mg TID have not been tested in well-controlled clinical efficacy studies due to the poor tolerability. Thus, there is a clear need for novel therapies that improve on the AE profile of pirfenidone while maintaining the anti-inflammatory and antifibrotic activity of pirfenidone for treatment of interstitial lung disease or other fibrotic-mediated pulmonary diseases and disorders.

### LYT-100 Pharmacology

As disclosed herein. LYT-100 retains the pharmacology of pirfenidone. Particularly, LYT-100 possesses anti-inflammatory and antifibrotic properties consistent with pirfenidone. Preclinical data disclosed herein demonstrate the antifibrotic and anti-inflammatory activity of LYT-100 (see, e.g., Examples 6-11). For instance, pretreatment with oral doses of 100 and 300 mg/kg LYT-100 inhibited TNFα and IL-6 in a rat lipopolysaccharide (LPS) model of systemic inflammation (Example 6), and LYT-100 at a dose of 60 mg/kg/day significantly reduced the area of liver fibrosis in a streptozocin-induced non-alcoholic steatohepatitis (NASH) mouse model (Example 7).

LYT-100 also reduced pro-inflammatory cytokines and suppressed TGF-β and downstream signaling to inhibit fibrosis in Primary Mouse Lung Fibroblasts (Example 8). Particularly, LYT-100 was found to: (i) reduce TGF-β-induced cell proliferation, (ii) reduce both background and TGF-β-induced levels of insoluble (structural) collagen; (iii) reduce both background and TGF-β-induced levels of soluble collagen; and (iv) reduce both background and TGF-β-induced levels of soluble fibronectin in primary mouse lung fibroblast (Example 8).

With reference to Example 9, LYT-100 (i) inhibits collagen synthesis, in the absence or presence TGF-β induction; (ii) inhibits total collagen levels in the absence or presence TGF-β induction; (iii) inhibits soluble collagen levels in the absence or presence TGF-β induction; and (iv) reduces soluble fibronectin levels in the absence and presence of TGF-β-induction.

Further, a DiscoverX BioMAP Fibrosis Panel was used to evaluate LYT-100 and pirfenidone as described in Example 5. Similar results were observed with both compounds in the three systems, indicating that the antifibrotic profile of pirfenidone is retained in LYT-100,

LYT-100 demonstrated activity in a mouse model of lymphederna (Example 10) and in a rat bleomycin-induced pulmonary fibrosis model (Example 11).

LYT-100 maintains the pharmacological profile of pirfenidone, and by virtue of the deuterium kinetic isotope effect on enzyme kinetics, has a differentiated pharmacokinetic profile relative to pirfenidone. Further, LYT-100 has an unexpectedly high tolerability, including a higher GI tolerability. The deuteration of pirfenidone to create LYT-100 slows its metabolism (Chen et al., Clinical Phar, in Drug Dev. 2021, 11(2), 220-234), and the altered metabolism may be associated with the reduced adverse effects and improved tolerability observed with LYT-100. allowing for higher dosing for greater effectiveness without the adverse effects seen at equivalent doses for pirfenidone. This discovery also allows for dosing without titration to immediately, and potentially more effectively, treat patients

Accordingly, in one aspect is provided a method of treating an interstitial lung disease or other fibrotic-mediated pulmonary disease or disorder, the method comprising administering to a subject in need thereof a total daily dose from about 825 mg to about 2550 mg of a deuterium-enriched pirfenidone having the structure:

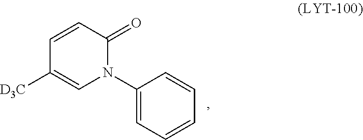 [SMILES: [2H]C([2H])([2H])c1ccc(=O)n(-c2ccccc2)c1]

wherein the interstitial lung disease or other fibrotic-mediated pulmonary disease or disorder is treated in the subject.

To arrive at the method disclosed herein (i.e., the dose range), numerous dose-ranging PK studies of LYT-100 were performed (e.g., several dose-ranging MAD studies ranging from total daily doses of 1000 mg to 4000 mg of LYT-100). PK modeling data incorporated the results of various MAD PK studies to reduce variability inherent in multiple studies of small sample size. The results of the pooled data from these various dose-ranging studies is shown below in Table 18 and indicated that 1) a dose of 550 mg TID LYT-100 had a systemic exposure (AUC) of about 90-98% (average about 95%) of the AUC achieved with pirfenidone (2403 mg dose, 801 mg TID) and a Cmax of about 73-80% (average about 77%) of the Cmax achieved with pirfenidone (2403 mg dose, 801 mg TID); and 2) a dose of 825 mg TID had a systemic exposure (AUC) of about 139-148% (average about 143%) of the AUC achieved with pirfenidone (2403 mg dose, 801 mg TID) and a Cmax of about 109-121% (average about 115%) of the Cmax achieved with pirfenidone (2403 mg dose, 801 mg TID). Notably, pirfenidone has not previously been tested for clinical efficacy above doses of 801 mg TID due to poor tolerability.

Table 1 summarizes the pharmacokinetic results of a cross-over study administering a dose of LYT-100 550 mg TID versus pirfenidone 801 mg TID. The results are expressed as Mean (SD), and shows that at the 550 mg TID dose, the AUC of LYT-100 is similar to that of pirfenidone dosed at the 801 mg TID dose while the Cₘₐₓ is lower. The AUC₀₋₂₄ of LYT-100 meets the criterion for bioequivalence (geometric mean ratio=0.875; 90% Confidence Interval=0.842 to 0.910) with pirfenidone 801 mg TID, while the Cₘₐₓ does not). The major metabolite of both pirfenidone and LYT-100, 5-carboxypirfenidone, showed lower Cₘₐₓ and AUC₀₋₂₄ after LYT-100 dosing at 550 mg TID compared to pirfenidone 801 mg TID. The reduced Cₘₐₓ of the parent and the 5-carboxypirfenidone with LYT-100 may be responsible for lowering the gastric side effects of pirfenidone while the similar level of total exposure (AUC) is expected to provide efficacy in interstitial lung disease and other fibrotic-mediated pulmonary diseases and disorders. Similar results were also seen on Day 4 or 14 after a single 550 mg dose of LYT-100 or 801 mg of pirfenidone was administered in the fasted state (Table 2). The Cₘₐₓ of the parent and the 5-carboxy metabolite were increased to a smaller extent after LYT-100 dosing than after pirfenidone dosing.

TABLE 1
Pharmacokinetic Parameters of LYT-100, Pirfenidone, and 5-Carboxypirfenidone
after the 3 Days of Dosing in the Fed State

| | LYT-100 550 mg TID PK | | Pirfenidone 801 mg TID PK | |
|---|---|---|---|---|
| | Parameters on Day 3/13 (Fed) | | Parameters on Day 3/13 (Fed) | |
| | Mean (SD) | | Mean (SD) | |
| | Cₘₐₓ | AUC₀₋₂₄ | Cₘₐₓ | AUC₀₋₂₄ |
| Analyte | (μg/mL) | (μg · hr/mL) | (μg/mL) | (μg · hr/mL) |
| Parent | 8.66 (3.0) | 131.4 (44.9) | 11.3 (4.8) | 155.2 (50.6) |
| 5-carboxy | 4.02 (1.0) | 67.19 (16.7) | 7.16 (2.5) | 108.2 (33.6) |
| pirfenidone | | | | |

TABLE 2
Pharmacokinetic Parameters of LYT-100, Pirfenidone, and 5-Carboxy-
pirfenidone after the 3 Days of Dosing in the Fed State Followed
by a Single Dose in the Fasted State or Day 4/14

| | LYT-100 550 mg TID PK | | Pirfenidone 801 mg TID PK | |
|---|---|---|---|---|
| | Parameters on Day 4/14 (Fasted) | | Parameters on Day 4/14 (Fasted) | |
| | Mean (SD) | | Mean (SD) | |
| | Cₘₐₓ | AUC₀₋₆ | Cₘₐₓ | AUC₀₋₆ |
| Analyte | (μg/mL) | (μg · hr/mL) | (μg/mL) | (μg · hr/mL) |
| Parent | 9.78 (3.0) | 36.43 (11.5) | 13.1 (3.2) | 45.92 (12.1) |
| 5-carboxy | 4.14 (1.2) | 16.83 (4.68) | 7.88 (2.4) | 29.84 (9.29) |
| pirfenidone | | | | |

In addition, further PK studies were performed to determine dosing frequency and dose amounts that were associated with improved tolerability (compared to the currently approved treatment of IPF, e.g., pirfenidone 801 mg TID). The dose that minimized AEs with a similar overall exposure level (AUC) to pirfenidone 801 mg TID was LYT-100 550 mg TID.

As shown in Table 3, LYT-100 550 mg TID and pirfenidone 801 mg TID PK and AE data were compared in the fed and fasted states (LYT-100-2021-103, Part 2). At the 550 mg TID (e.g., similar drug exposure level to approved 801 TID pirfenidone), lower AEs were observed with LYT-100 in both the fed and fasted states compared with pirfenidone. Specifically, administering a daily dose of 1650 mg LYT-100 demonstrated that LYT-100 550 mg TID was associated with improved tolerability compared to pirfenidone, including a 50% reduction in gastrointestinal-related AEs and a 45% reduction in CNS-related AEs (see Example 1 and Results for LYT-100-2021-103 Part 2, shown in Table 3).

Although the AEs observed with the administration of 550 mg TID LYT-100 in the fasted state were higher than the AEs seen in the fed state, the AEs with LYT-100 550 mg TID in the fasted state were still much lower than those seen with pirfenidone 801 mg TID in the fasted state. These results demonstrate that, at the same/similar drug exposure level of 801 TID pirfenidone, LYT-100 administered 550 TID has improved tolerability (less AEs) and the option of being given in the fasted state if needed, such as with individual variation in timing of meals. These data provide the rationale for selecting the 550 mg TID dose of LYT-100 in the treatment of interstitial lung disease and other fibrotic-mediated pulmonary diseases and disorders. When rates of AEs were ordered from lowest to highest, Cmax values for parent compound for each of these conditions similarly sorted from lowest to highest: Lowest AE rates and Cmax to highest AEs and Cmax=LYT-100 550 mg (fed) to LYT-100 550 mg (fasted) to pirfenidone 801 mg (fed) to pirfenidone 801 mg (fasted)—Table 3 (LYT-100-2021-103 Part 2). Levels of the 5-carboxy metabolite also sorted from lowest to highest in the above order (Table 3) (LYT-100-2021-103 Part 2).

TABLE 3
LYT-100 550 mg TID and pirfenidone 801 mg TID, fed and fasted, PK and AE data

| | LYT-100 | | Pirfenidone | | LYT-100 | | Pirfenidone | |
|---|---|---|---|---|---|---|---|---|
| | 550 mg TID | | 801 mg TID | | 550 mg TID | | 801 mg TID | |
| | Fed | | Fed | | Fasted | | Fasted | |
| | N = 46 PK | | N = 47 PK | | N = 44 PK | | N = 47 PK | |
| | Cₘₐₓ | AUC₀₋₂₄ | Cₘₐₓ | AUC₀₋₂₄ | Cₘₐₓ | AUC₀₋₆ | Cₘₐₓ | AUC₀₋₆ |
| | (mcg/mL) | mcg*hr/mL | (mcg/mL) | mcg*hr/mL | (mcg/mL) | mcg*hr/mL | (mcg/mL) | mcg*hr/mL |
| Pharmacokinetics | Mean (SD) | | Mean (SD) | | Mean (SD) | | Mean (SD) | |
| PK Parent | 8.66 | 131.4 | 11.3 | 155.2 | 9.78 | 36.43 | 13.1 | 45.92 |
| | (3.0) | (44.9) | (4.8) | (50.6) | (3.0) | (11.5) | (3.2) | (12.1) |
| PK 5-carboxy | 4.02 | 67.19 | 7.16 | 108.2 | 4.14 | 16.83 | 7.88 | 29.84 |
| metabolite | (1.0) | (16.7) | (2.5) | (33.6) | (1.2) | (4.68) | (2.4) | (9.29) |
| Adverse Events* | N = 46 | | N = 47 | | N = 45 | | N = 46 | |
| | n (%) | | n (%) | | n (%) | | n (%) | |
| GI Disorders | 3 (6.5) | | 5 (10.6) | | 5 (11.1) | | 13 (28.3) | |
| Nausea | 2 (4.3) | | 4 (8.5) | | 5 (11.1) | | 12 (26.1) | |
| Vomiting | 2 (4.3) | | 2 (4.3) | | 0 | | 1 (2.2) | |
| Diarrhea | 0 | | 0 | | 0 | | 0 | |
| Dyspepsia | 0 | | 0 | | 0 | | 0 | |
| Abdominal Pain/ | 1 (2.2) | | 1 (2.2) | | 0 | | 2 (4.3) | |
| Discomfort/ | | | | | | | | |
| Distension | | | | | | | | |
| Nervous System | 4 (8.7) | | 7 (14.9) | | 4 (8.9) | | 10 (21.7) | |
| Disorders | | | | | | | | |

| | | | | |
|---|---|---|---|---|
| Headache | 4 (8.7) | 5 (10.6) | 2 (4.4) | 5 (10.9) |
| Dizziness | 0 | 3 (6.4) | 1 (2.2) | 5 (10.9) |

Table 4 summarizes the pharmacokinetic results and shows that at the 550 mg TID dose, the PK parameters of LYT-100 and the metabolite, 5-carboxypirfenidone were similar to those seen in Part 2 of the study at the 550 mg TID dose of LYT-100. At the higher dose of 824 mg TID, the AUC₀₋₂₄ and Cₘₐₓ were higher than those seen with pirfenidone; however, the corresponding parameters of the metabolite 5-carboxypirfenidone were similar/slightly lower. The adverse event data (Table 6) shows that even at the 824 mg TID dose, the frequency of the most common adverse events was very low. The higher exposures combined with low frequency of adverse events provide the rationale for using the 825 mg TID dose of LYT-100 in the treatment of interstitial lung disease and other fibrotic-mediated pulmonary diseases and disorders.

TABLE 4
Pharmacokinetic Parameters of LYT-100, Pirfenidone, and 5-Carboxypirfenidone
after the 3 Days of Dosing in the Fed State

| | LYT-100 550 mg TID | | LYT-100 824 mg TID | |
|---|---|---|---|---|
| | PK Parameters on Day 3 (Fed) | | PK Parameters on Day 6 (Fed) | |
| | N = 13, Mean (SD) | | N = 13, Mean (SD) | |
| | Cₘₐₓ | AUC₀₋₂₄ | Cₘₐₓ | AUC₀₋₂₄ |
| Analyte | (μg/mL) | (μg · hr/mL) | (μg/mL) | (μg · hr/mL) |
| Parent | 9.34 (2.4) | 132.0 (31.2) | 13.7 (5.1) | 193.9 (60.6) |
| 5-carboxy | 4.74 (2.0) | 69.12 (22.9) | 6.75 (2.6) | 95.24 (32.3) |
| pirfenidone | | | | |

The dose of LYT-100 was optimized to achieve similar systemic exposure (AUC) to pirfenidone 801 mg TID. The dose of LYT-100 was also optimized to achieve similar Cmax to pirfenidone 801 mg TID while maximizing exposure (AUC). The Cmax and AUC values obtained using the pooled LYT-100 PK data in comparison with 801 mg TID pirfenidone were confirmed in subsequent individual studies of 550 mg TID LYT-100 and 824 mg TID LYT-100, thus confirming our confidence in the modeling data and the use of 550 mg TID and 825 mg TID LYT-100 doses.

The dose of LYT-100 was optimized to achieve similar Cmax to pirfenidone 801 mg TID while maximizing drug exposure (AUC). Study LYT-100-2021-103 Part 3 was a randomized, double-blinded, parallel arm, placebo-controlled study conducted in healthy older adults to evaluate the safety and tolerability of titrated high dose LYT-100 compared to placebo under fed conditions. Based on the observations of improved tolerability (but comparable total exposure) for a lower TID dose of LYT-100 compared to pirfenidone in Part 2 (550 TID LYT-100), the decision was made to test the safety and tolerability of a higher TID dose of LYT-100, to achieve a higher overall predicted AUC or total exposure than the approved dose of pirfenidone (801 mg TID). Subjects between the ages of 60 and 80 were randomized to receive LYT-100 or placebo. Subjects were administered up to 550 mg LYT-100 TID for 3 days (to steady state [Day 1 to Day 3]) compared to placebo administered TID for 3 days to steady state. On Day 4 to Day 6, subjects were administered 824 mg LYT-100 TID for 3 days compared to placebo TID for 3 days to steady state. A summary of the dosing scheme is provided below in the Example section (Example 2).

Table 5 summarizes the pharmacokinetic results and shows that at the 550 mg TID dose, the PK parameters of LYT-100 and the metabolite, 5-carboxypirfenidone were similar to those seen in Part 2 of the study at the 550 mg TID dose of LYT-100. At the higher dose of 824 mg TID, the AUC₀₋₂₄ and Cₘₐₓ were higher than those seen with pirfenidone 801 mg TID; however, the corresponding parameters of the metabolite 5-carboxypirfenidone were similar or slightly lower. The adverse event data (Table 6) shows that even at the 824 mg TID dose, the frequency of the most common adverse events was very low. The higher exposures combined with low frequency of adverse events provide the rationale for using the 825 mg TID dose of LYT-100 in treating interstitial lung disease and other fibrotic-mediated pulmonary diseases and disorders.

TABLE 5
Pharmacokinetic Parameters of LYT-100, Pirfenidone, and 5-Carboxypirfenidone
after the 3 Days of Dosing in the Fed State

| | LYT-100 550 mg TID | | LYT-100 824 mg TID | |
|---|---|---|---|---|
| | PK Parameters on Day 3 (Fed) | | PK Parameters on Day 6 (Fed) | |
| | N = 13, Mean (SD) | | N = 13, Mean (SD) | |
| | Cₘₐₓ | AUC₀₋₂₄ | Cₘₐₓ | AUC₀₋₂₄ |
| Analyte | (μg/mL) | (μg · hr/mL) | (μg/mL) | (μg · hr/mL) |
| Parent | 9.34 (2.4) | 132.0 (31.2) | 13.7 (5.1) | 193.9 (60.6) |
| 5-carboxy | 4.74 (2.0) | 69.12 (22.9) | 6.75 (2.6) | 95.24 (32.3) |
| pirfenidone | | | | |

LYT-100 824 mg TID achieved approximately 25% higher AUC with a modestly higher Cmax compared to historic pirfenidone PK values. Surprisingly, as shown in Table 6, this high dose of LYT-100 (825 mg TID) was well-tolerated. Prior to completing the tolerability study shown in Table 6, it was not known such high dose—825 mg TID LYT-100 which is the equivalent of about 120-150% exposure of 801 TID pirfenidone)—could be sufficiently tolerated to be included in a clinical efficacy study.

TABLE 6
Pharmacokinetic parameters and adverse events
for LYT-100 and 5-carboxypirfenidone metabolite

| | Healthy Older Part 3 | | | |
|---|---|---|---|---|
| | LYT-100 | | LYT-100 | |
| | 550 mg TID | | 824 mg TID | |
| | Fed | | Fed | |
| | N = 13 | | N = 13 | |
| | Cₘₐₓ | AUC₀₋₂₄ | Cₘₐₓ | AUC₀₋₂₄ |
| | (mcg/mL) | mcg*hr/mL | (mcg/mL) | mcg*hr/mL |
| Pharmacokinetics | Mean (SD) | | Mean (SD) | |
| PK Parent | 9.34 (2.4) | 132.0 (31.2) | 13.7 (5.1) | 193.9 (60.6) |
| PK 5-carboxy | 4.74 (2.0) | 69.12 (22.9) | 6.75 (2.6) | 95.24 (32.3) |
| metabolite | | | | |
| Adverse Events* | N = 13 | | N = 13 | |
| | n (%) | | n (%) | |
| GI Disorders | 2 (8.3) | | 0 | |
| Nausea | 0 | | 0 | |
| Vomiting | 0 | | 0 | |
| Diarrhea | 1 (4.2) | | 0 | |
| Dyspepsia | 1 (4.2) | | 0 | |
| Abdominal Pain/ | 0 | | 0 | |
| Discomfort/ | | | | |
| Distension | | | | |
| Nervous System | 3 (12.5) | | 0 | |
| Disorders | | | | |

| | | |
|---|---|---|
| Headache | 3 (12.5) | 0 |
| Dizziness | 1 (4.2) | 0 |

*AEs reported are those that are most common for pirfenidone and seen across LYT-100 studies including GI and Nervous System Disorders

Table 7 summarizes the pharmacokinetic results for the 550 mg TID doses and the 825 mg TID, along with the observed adverse events and frequency, and further provides comparative data for pirfenidone in a related study.

TABLE 7
Pharmacokinetic parameters and adverse events for LYT-
100, pirfenidone, and 5-carboxypirfenidone metabolite

| | | | | | Healthy Older Adult Part 2 (pirf. | |
|---|---|---|---|---|---|---|
| | Healthy Older Adult Part 3 | | | | Arm- for comparison only) | |
| | LYT-100 | | LYT-100 | | Pirfenidone | |
| | 550 mg TID | | 824 mg TID | | 801 mg TID | |
| | Fed | | Fed | | Fed | |
| | N = 13 | | N = 13 | | N = 47 PK | |
| | Cₘₐₓ | AUC₀₋₂₄ | Cₘₐₓ | AUC₀₋₂₄ | Cₘₐₓ | AUC₀₋₂₄ |
| | (mcg/mL) | mcg*hr/mL | (mcg/mL) | mcg*hr/mL | (mcg/mL) | mcg*hr/mL |
| Pharmacokinetics | Mean (SD) | | Mean (SD) | | Mean (SD) | |
| PK Parent | 9.34 | 132.0 | 13.7 | 193.9 | 11.3 | 155.2 |
| | (2.4) | (31.2) | (5.1) | (60.6) | (4.8) | (50.6) |
| PK 5-carboxy | 4.74 | 69.12 | 6.75 | 95.24 | 7.16 | 108.2 |
| metabolite | (2.0) | (22.9) | (2.6) | (32.3) | (2.5) | (33.6) |
| Adverse Events* | N = 13 | | N = 13 | | N = 47 | |
| | n (%) | | n (%) | | n (%) | |
| GI Disorders | 2 (8.3) | | 0 | | 5 (10.6) | |
| Nausea | 0 | | 0 | | 4 (8.5) | |
| Vomiting | 0 | | 0 | | 2 (4.3) | |
| Diarrhea | 1 (4.2) | | 0 | | 0 | |
| Dyspepsia | 1 (4.2) | | 0 | | 0 | |
| Abdominal Pain/ | 0 | | 0 | | 1 (2.2) | |
| Discomfort/ | | | | | | |
| Distension | | | | | | |

| Nervous System | 3 (12.5) | 0 | 7 (14.9) |
|---|---|---|---|
| Disorders | | | |

| | | | |
|---|---|---|---|
| Headache | 3 (12.5) | 0 | 5 (10.6) |
| Dizziness | 1 (4.2) | 0 | 3 (6.4) |

*AEs reported are those that are most common for pirfenidone and seen across LYT-100 studies inc. GI and Nervous System Disorders

The 550 mg TID and 825 mg TID doses of LYT-100 were optimized to key PK parameters and demonstrated to improve tolerability as compared with 2304 mg daily dose (801 mg TID) pirfenidone, surprisingly even at a higher systemic drug exposure. This improved tolerability of LYT-100 relative to pirfenidone was unexpected and may significantly improve compliance with a sustained high efficacious dose (e.g., by reducing the frequency of dose reductions, treatment interruptions, and/or temporary or permanent discontinuations experienced with the use of pirfenidone).

Overall, as described in Examples 1 and 2 of the present disclosure, in Parts 1 and 2 of the clinical study, 850 mg BID of LYT-100 closely matched the AUC with slightly higher Cₘₐₓ with pirfenidone 801 mg TID; and 550 mg TID LYT-100 dose matched the AUC (within BE) with lower Cₘₐₓ compared to pirfenidone 801 mg TID. With continued reference to the Examples, in the first study, on day 3, LYT-100 550 mg TID dosed in the fed state, had a 24% lower Cₘₐₓ with a similar AUC versus fed pirfenidone 801 mg TID; on day 4, both pirfenidone and 550 mg TID LYT-100, dosed in the fasted state, resulted in higher drug exposures, with a larger Cₘₐₓ increase for pirfenidone; on day 3, in the fed state, the metabolite Cₘₐₓ was 46% lower for LYT-100 vs pirfenidone; and AE rates trended with Cₘₐₓ of parent and metabolite (for fasted pirfenidone, fed pirfenidone, fasted LYT-100, and fed LYT-100, the AE rates respectively, were: GI 28.3%, 10.6%, 11.1%, 6.5%; CNS 21.7%, 14.9%, 8.9%, 8.7%). With continued reference to the Examples, in the second study, on day 3, LYT-100 dosed at 824 mg TID had a Cₘₐₓ 57% higher and an AUC₀₋₂₄ 43% higher than those for LYT-100 dosed at 550 mg. The AEs were low and comparable between LYT-100 824 mg TID and placebo (GI 0%, CNS 0%, 16.7% infection (COVID-19) for both arms).

With further reference to the Examples, results of Part 3 of the Study were unexpected given the predictions based on Study Parts 1 and 2). Specifically, 1) LYT-100 550 mg TID had much lower AUC but similar Cmax compared to Part 2; 2) LYT-100 824 mg TID had lower AUC than predicted; Cmax was 17% higher than with pirfenidone; 3) although higher variability was seen in PK parameters of LYT-100 in Part 3, the Metabolite/Parent Ratio was consistently lower with LYT-100 compared to pirfenidone (i.e., the 5-carboxy metabolite exposures are lower when comparing the same doses of LYT-100 and pirfenidone); 4) the GI AE's and nausea are much lower with LYT-100 (550 mg TID) compared to pirfenidone 801 mg TID; 5) dosing in the fed state lowered the GI-related AE's, especially for pirfenidone, but had less of an impact on AEs with LYT-100; 6) the GI AE's appear early during treatment; and 7) better tolerability of LYT-100 at the 550 mg TID dose allows subjects to have better adherence with the full dose, which may result in a better clinical outcome in various interstitial lung disease and other fibrotic-mediated pulmonary diseases and disorders.

In the disclosed method, the dose and frequency of dosing may vary based on the diseases or disorder and the severity thereof, as well as on the desired pharmacokinetic parameters and tolerability profile. Particularly, the improved tolerability of LYT-100 (e.g., less adverse side effects) can allow dosing at therapeutic (efficacious) levels, e.g., including dosing at the current approved therapeutic dose for pirfenidone, with less or no treatment interruption, less or no treatment discontinuation, less or no dose-lowering in treating interstitial lung disease and other fibrotic-mediated pulmonary diseases or disorders. This greater tolerability of deuterium-enriched pirfenidone LYT-100 can allow for sustained or long-term treatment at therapeutic dosing resulting in effective treatment of patients afflicted with a variety of interstitial lung diseases and other fibrotic-mediated pulmonary diseases and disorders. The improved tolerability also provides the potential for dosing without titration or with a reduced duration of titration, to more rapidly and effectively treat patients with a variety of interstitial lung diseases and other fibrotic-mediated pulmonary diseases and disorders. The improved tolerability also provides the potential for higher dosing (systemic exposure) for greater effectiveness without the adverse effects seen at equivalent doses (systemic exposure) for pirfenidone.

As described above, the method generally comprises administering LYT-100 at a total daily dose from about 825 mg to about 2550 mg of LYT-100. In some embodiments, LYT-100 is administered at a daily dose that achieves the same or about the same systemic exposure as pirfenidone administered at a dose of 2403 mg/day. In some embodiments, the method comprises administering a total daily dose of LYT-100 that achieves a systemic exposure greater than the systemic exposure of pirfenidone dosed at 2403 mg daily dose, e.g., 801 mg TID dosing.

In some embodiments, the total daily dose is from about 825 to about 1650 mg, such as about 825, about 1100, about 1375, or about 1650 mg. In some embodiments, the total daily dose in 825 mg.

In some embodiments, the total daily dose is from about 1650 to about 2550 mg of LYT-100, such as about 1650, about 1700, about 1750, about 1800, about 1850, about 1900, about 1950, about 2000, about 2050, about 2100, about 2150, about 2200, about 2250, about 2300, about 2350, about 2400, about 2450, about 2475, about 2500, or about 2550 mg. In some embodiments, the total daily dose is from about 1650 mg to about 2475 mg. In some embodiments, the total daily dose is 1650 mg. In some embodiments, the total daily dose is 2475 mg.

In some embodiments, the total daily dose is administered in three equal administrations. In some embodiments, the LYT-100 is administered in three equal doses of 550 mg each (550 mg TID). In some embodiments, the LYT-100 is administered in three equal doses of 825 mg each (825 mg TID). In some embodiments, the LYT-100 is administered in three equal doses of 275 mg each (275 mg TID).

In some embodiments, the LYT-100 is administered without regard to food. In some embodiments, the LYT-100 is administered without food. In some embodiments, the LYT-100 is administered with food.

In some embodiments, the LYT-100 is administered orally without food in three daily doses of 550 mg each. In some embodiments, the LYT-100 is administered orally with food in three daily doses of 550 mg each.

In some embodiments, the LYT-100 is administered orally without food in three daily doses of 825 mg each. In some embodiments, the LYT-100 is administered orally with food in three daily doses of 825 mg each.

In some embodiments, the LYT-100 is administered orally without food in three daily doses of 275 mg each. In some embodiments, the LYT-100 is administered orally with food in three daily doses of 275 mg each.

In some embodiments, the LYT-100 is administered without dose escalation. In some embodiments, the LYT-100 is administered in three equal administrations of 550 mg each, without dose escalation. In some embodiments, the LYT-100 is administered in three equal administrations of 825 mg each, without dose escalation.

In some embodiments, the LYT-100 is administered with dose escalation. In some embodiments, three daily doses of 550 mg each are administered for three days, followed by administering LYT-100 at a dosage of 825 mg TID. Referring to the crossover study described in Example 2, initial data for the occurrence of adverse events in healthy elderly subjects taking doses of 550 mg TID (1650 mg/day) followed by 824 TID (2472 mg/day) indicates that adverse events (particularly gastrointestinal (GI) disorders and nervous system disorders) do not increase and may decrease or even disappear with this dose titration scheme.

In some embodiments, administering comprises titrating up to the total daily dose from an initial total daily dose which is below the total daily dose, and wherein titrating comprises administering the LYT-100 in three daily doses of 550 mg each for an initial period of time, followed by administering the LYT-100 in three daily doses of 825 mg each for a period of time. In some embodiments, the titrating comprises administering LYT-100 in three daily doses of 275 mg each for an initial period of time, followed by administering the the LYT-100 in three daily doses of 550 mg each for a period of time, optionally followed by administering the LYT-100 in three daily doses of 825 mg each for a period of time. In some embodiments, the initial period of time is 3-14 days. In some embodiments, the initial period of time is 3-7 days.

In comparison, as reported in regulatory summaries leading to approval of Esbriet (pirfenidone), escalating daily doses (801, 1602, 2403, 3204, and 4005 mg/day, provided in three equal doses) of pirfenidone were tested in a cohort of healthy older subjects (PIPF-005). The number of AEs (headache, dyspepsia, nausea, back pain) reported increased with increasing total daily dose. The higher Cₘₐₓ values at higher dosages increased the odds of experiencing a gastrointestinal (GI) AE, and it was noted that this was consistent with previous studies for pirfenidone. As reported for study PIPF-005, for the three times daily dose of 801 mg (2403 mg/day), the Cₘₐₓ was 11.85 μg/mL, which falls between the Cₘₐₓ values reported for 550 mg TID and 824 mg TID in Example 2.

In some embodiments, the method comprises administering LYT-100 at a first total daily dose of 825 mg for a first period and a second total daily maintenance dose of 1650 mg. In some embodiments, the method comprises administering LYT-100 at a first total daily dose of 1650 mg for a first period and a second total daily maintenance dose of 2475 mg. In some embodiments, the method comprises administering LYT-100 at a first total daily dose of 825 mg for a first period, a second total daily dose of 1650 mg for a second period, and then a total maintenance dose of 2475 mg. In some embodiments, the method comprises administering LYT-100 at a first total daily dose of 825 mg for a first period of about 7 days and a second total daily maintenance dose of 1650 mg. In some embodiments, the method comprises administering LYT-100 at a first total daily dose of 1650 mg for a first period of about 7 days and a second total daily maintenance dose of 2475 mg. In some embodiments, the method comprises administering LYT-100 at a first total daily dose of 825 mg for a first period of about 7 days, a second total daily dose of 1650 mg for a second period of about 7 days, and then a total maintenance dose of 2475 mg. In some embodiments, the method comprises administering LYT-100 at a first total daily dose of 825 mg for a first period of about 14 days and a second total daily maintenance dose of 1650 mg. In some embodiments, the method comprises administering LYT-100 at a first total daily dose of 1650 mg for a first period of about 14 days and a second total daily maintenance dose of 2475 mg. In some embodiments, the method comprises administering LYT-100 at a first total daily dose of 825 mg for a first period of about 14 days, a second total daily dose of 1650 mg for a second period of about 14 days, and then a total maintenance dose of 2475 mg. In some embodiments, the method comprises administering LYT-100 at a first total daily dose of 825 mg for a first period of 7-14 days and a second total daily maintenance dose of 1650 mg. In some embodiments, the method comprises administering LYT-100 at a first total daily dose of 1650 mg for a first period of 7-14 days and a second total daily maintenance dose of 2475 mg. In some embodiments, the method comprises administering LYT-100 at a first total daily dose of 825 mg for a first period of 7-14 days, a second total daily dose of 1650 mg for a second period of 7-14 days, and then a total maintenance dose of 2475 mg.

In some embodiments, the method comprises administering LYT-100 in three daily doses of 275 mg each for a first period and in three daily doses of 550 mg each for a second maintenance dose. In some embodiments, the method comprises administering LYT-100 in three daily doses of 550 mg each for a first period and in three daily doses of 825 mg each for a second maintenance dose. In some embodiments, the method comprises administering LYT-100 in three daily doses of 275 mg each for a first period, in three daily doses of 550 mg each for a second period, and then in three daily doses of 825 mg each for a maintenance dose. In some embodiments, the method comprises administering LYT-100 in three daily doses of 275 mg each for a first period of about 7 days and in three daily doses of 550 mg each for a second maintenance dose. In some embodiments, the method comprises administering LYT-100 in three daily doses of 550 mg each for a first period of about 7 days and in three daily doses of 825 mg each for a second maintenance dose. In some embodiments, the method comprises administering LYT-100 in three daily doses of 275 mg each for a first period of about 7 days, in three daily doses of 550 mg each for a second period of about 7 days, and then in three daily doses of 825 mg each for a maintenance dose. In some embodiments, the method comprises administering LYT-100 in three daily doses of 275 mg each for a first period of about 14 days and in three daily doses of 550 mg each for a second maintenance dose. In some embodiments, the method comprises administering LYT-100 in three daily doses of 550 mg each for a first period of about 14 days and in three daily doses of 825 mg each for a second maintenance dose. In some embodiments, the method comprises administering LYT-100 in three daily doses of 275 mg each for a first period of about 14 days, in three daily doses of 550 mg each for a second period of about 14 days, and then in three daily doses of 825 mg each for a maintenance dose. In some embodiments, the method comprises administering LYT-100 in three daily doses of 275 mg each for a first period of 7-14 days and in three daily doses of 550 mg each for a second maintenance dose. In some embodiments, the method comprises administering LYT-100 in three daily doses of 550 mg each for a first period of 7-14 days and in three daily doses of 825 mg each for a second maintenance dose. In some embodiments, the method comprises administering LYT-100 in three daily doses of 275 mg each for a first period of 7-14 days, in three daily doses of 550 mg each for a second period of 7-14 days, and then in three daily doses of 825 mg each for a maintenance dose.

In any of the above embodiments, the LYT-100 is administered orally without food. In any of the above embodiments, the LYT-100 is administered orally with food. In any of the above embodiments, the LYT-100 is administered orally without regard to food. In any of the above embodiments, the total daily dose, e.g., 825 mg, 1650 mg or 2475 mg may be adjusted to lower daily dose, for example, as described elsewhere in the specification.

In some embodiments, LYT-100 administered in a total daily dose of 1650-2475, in three daily doses, results in increased tolerability as compared with pirfenidone administered at 801 mg TID. In some embodiments, the increased tolerability is due to a reduction in one or more adverse events or side effects. In some embodiments, the one or more adverse events are nervous system side effects. In some embodiments, the one or more adverse events are gastrointestinal events. In some embodiments, the LYT-100 is administered in three daily doses of 550 mg each.

In some embodiments, LYT-100 administered in a total daily dose of 1650-2475, in three daily doses, results in a lower steady-state Cₘₐₓ as compared with pirfenidone administered at 801 mg TID. In some embodiments, the LYT-100 is administered in three daily doses of 550 mg each.

In some embodiments, LYT-100 administered in a total daily dose of 1650-2475, in three daily doses, results in a steady-state exposure (AUC) of LYT-100 which is the same or about the same as the steady-state exposure (AUC) of pirfenidone achieved when pirfenidone is administered at 801 mg TID. In some embodiments, LYT-100 administered in a total daily dose of 1650-2475, in three daily doses, results in a steady-state exposure (AUC) of LYT-100 which is bioequivalent to the steady-state exposure (AUC) of pirfenidone when pirfenidone is administered at 801 mg TID. In some embodiments, the LYT-100 is administered in three daily doses of 550 mg each.

In some embodiments, LYT-100 administered in a total daily dose of 1650-2475, in three daily doses, results in the same or about the same steady-state exposure (AUC) of LYT-100 achieved for pirfenidone when pirfenidone is administered at 801 mg TID, and results in a lower steady-state Cₘₐₓ of LYT-100 achieved for pirfenidone when pirfenidone is administered at 801 mg TID. In some embodiments, the steady-state exposure of LYT-100 is about 90% of the AUC of pirfenidone achieved when pirfenidone is administered at a total daily dose of 2403 mg, and wherein the total daily dose of pirfenidone is administered in three doses of 801 mg each (801 mg TID). In some embodiments, the lower steady-state Cₘₐₓ of LYT-100 is about 75-80% of the Cₘₐₓ of pirfenidone achieved when pirfenidone is administered at a total daily dose of 2403 mg, and wherein the total daily dose of pirfenidone is administered in three doses of 801 mg each (801 mg TID). In some embodiments, at this dosing, the LYT-100 has an increased or improved tolerability that is due to a reduction in one or more adverse events or side effects as compared with pirfenidone administered at 801 mg TID. In some embodiments, the LYT-100 is administered in three daily doses of 550 mg each.

In some embodiments, LYT-100 administered in a total daily dose of 1650-2475, in three daily doses, results in the same or about the same steady-state exposure (AUC) as compared with pirfenidone administered at 801 mg TID and increased or improved tolerability as compared with pirfenidone administered at 801 mg TID. In some embodiments, the increased or improved tolerability is due to a reduction in one or more adverse events or side effects. In some embodiments, LYT-100 is administered in three daily doses of 550 mg each.

In some embodiments, LYT-100 administered in a total daily dose of 1650-2475, in three daily doses, results in a higher steady-state exposure (AUC) as compared with pirfenidone administered at 801 mg TID. In some embodiments, the LYT-100 is administered in three daily doses of 825 mg each.

In some embodiments, LYT-100 administered in a total daily dose of 1650-2475, in three daily doses, results in the same or about the same steady-state Cmax as compared with pirfenidone administered at 801 mg TID. In some embodiments, the LYT-100 is administered in three daily doses of 825 mg each.

In some embodiments, LYT-100 administered in a total daily dose of 1650-2475, in three daily doses, results in a higher steady-state exposure (AUC) as compared with pirfenidone administered at 801 mg TID and the same or about the same steady-state Cₘₐₓ as compared with pirfenidone administered at 801 mg TID. In some embodiments, the LYT-100 is administered in three daily doses of 825 mg each.

In some embodiments, LYT-100 administered in a total daily dose of 1650-2475, in three daily doses, results in a higher steady-state exposure (AUC) as compared with pirfenidone administered at 801 mg TID and has the same or about the same tolerability (e.g., the incidence of adverse events is not significantly different) as compared with pirfenidone administered at 801 mg TID. In some embodiments, LYT-100 administered in a total daily dose of 1650-2475, in three daily doses, results in a higher steady-state exposure (AUC) as compared with pirfenidone administered at 801 mg TID and has an increased or improved tolerability that is due to a reduction in one or more adverse events or side effects. In some embodiments, the LYT-100 is administered in three daily doses of 825 mg each.

In some embodiments, LYT-100 administered in a total daily dose of 1650-2475, in three daily doses, results in the same or about the same steady-state Cmax as compared with pirfenidone administered at 801 mg TID and has the same or about the same tolerability (e.g., the incidence of adverse events is not significantly different) as compared with pirfenidone administered at 801 mg TID. In some embodiments, LYT-100 administered in a total daily dose of 1650-2475, in three daily doses, results in the same or about the same steady-state Cmax as compared with pirfenidone administered at 801 mg TID and has an increased or improved tolerability that is due to a reduction in one or more adverse events or side effects. In some embodiments, the LYT-100 is administered in three daily doses of 825 mg each.

In some embodiments, the LYT-100 is administered at a dose that achieves a systemic exposure of LYT-100 in the subject which is about 85-125% of the systemic exposure of pirfenidone achieved when pirfenidone is administered at a total daily dose of 2403 mg, and wherein the total daily dose of pirfenidone is administered in three doses of 801 mg each (801 mg TID).

In some embodiments, the dose of LYT-100 that achieves the systemic exposure of LYT-100 in the subject which is about 85-125% of the systemic exposure of pirfenidone is 825 mg TID.

In some embodiments, the dose of LYT-100 that achieves the systemic exposure of LYT-100 in the subject which is about 85-125% of the systemic exposure of pirfenidone also achieves a Cₘₐₓ of LYT-100 in the subject which is about 115-125% of the Cₘₐₓ of pirfenidone achieved when pirfenidone is administered at a total daily dose of 2403 mg, and wherein the total daily dose of pirfenidone is administered in three doses of 801 mg each (801 mg TID). In some embodiments, at this dosing, the LYT-100 has the same or about the same tolerability (e.g., the incidence of adverse events is not significantly different) as compared with pirfenidone administered at 801 mg TID. In some embodiments, at this dosing, the LYT-100 has an increased or improved tolerability that is due to a reduction in one or more adverse events or side effects as compared with pirfenidone administered at 801 mg TID. In some embodiments, the LYT-100 is administered in three daily doses of 825 mg each.

As described above, in some embodiments, administration of LYT-100 according to the disclosed method results in increased or improved tolerability that is due to a reduction in one or more adverse events or side effects in a subject as compared with pirfenidone administered at a dose or dosing that achieves the same or about the same systemic exposure.

In any of these embodiments wherein one or more side effects is reduced, the incidence, the severity, ot both may be reduced. In some embodiments, the incidence (i.e., the frequency with which side effects occur) of side effects in an individual patient or in a patient population, is reduced by at least 30% as compared with pirfenidone administered at a dose or dosing that achieves the same or about the same systemic exposure. For example, in some embodiments, the incidence of side effects is reduced by at least 35%, at least 40%, or at least about 50% as compared with pirfenidone administered at a dose or dosing that achieves the same or about the same systemic exposure. In some embodiments, the incidence of side effects is reduced by at least 30% as compared with pirfenidone administered at a total daily dose of 2403 mg, including e.g., at 801 mg TID.

In some embodiments, the incidence of side effects is reduced when the subject is dosed in a fed state as compared with pirfenidone administered at a dose or dosing that achieves the same or about the same systemic exposure in a fed state. In some embodiments, the incidence of side effects is reduced when the subject is dosed in a fasted state as compared with pirfenidone administered at a dose or dosing that achieves the same or about the same systemic exposure in a fasted state.

In some embodiments, the one or more side effects is a gastrointestinal side effect(s). In some embodiments, the one or more side effects is a nervous system side effect(s). In some embodiments, the one or more side effects is a combination of gastrointestinal side effect(s) and nervous system side effect(s). Examples of gastrointestinal side effects include nausea, vomiting, and abdominal pain or distension.

In some embodiments, the nervous system and/or gastrointestinal side effects in a subject are reduced with administration of LYT-100 at a total daily dose of 1650 mg, optionally wherein the LYT-100 is administered TID. In some embodiments, the nervous system and/or gastrointestinal side effects in a subject are reduced with administration of LYT-100 at a total daily dose of 2475 mg, optionally wherein the LYT-100 is administered TID.

Interstitial Lung Diseases and other Fibrotic-Mediated Pulmonary Diseases and Disorders The disclosed method generally treats interstitial lung diseases and other fibrotic-mediated pulmonary diseases and disorders diseases and disorders. Accordingly, the method may treat a variety of diseases and disorders of a fibrotic and/or inflammatory nature. In some embodiments, the interstitial lung disease or other fibrotic-mediated pulmonary disease or disorder, or a symptom thereof, is alleviated. In some embodiments, the onset of the interstitial lung disease or other fibrotic-mediated pulmonary disease or disorder is delayed, slowed, or arrested. In some embodiments, the progression of the interstitial lung disease or other fibrotic-mediated pulmonary disease or disorder is delayed, slowed, or arrested.

### Interstitial Lung Diseases and Other Fibrotic-Mediated Pulmonary Disorders

In some embodiments, the method treats an interstitial lung disease (ILD). ILDs encompasses a large and heterogeneous group of pulmonary disorders which overlap in their clinical presentations and patterns of lung injury. ILDs are generally characterized by the disruption of the distal lung parenchyma. resulting in alteration of the interstitial space, which leads to clinical symptoms such as dyspnea and cough, and results in restrictive ventilatory and gas exchange deficits on pulmonary function testing. ILDs include several diseases of unknown cause, as well as ILDs known to be related to other diseases or to environmental exposures. Although the cause of many ILDs is not known, the disease typically involves some form of injury to the alveolar epithelial cells initiating an inflammatory response coupled with repair mechanisms. The injury-repair process is reflected pathologically as inflammation, fibrosis or a combination of both. Common characteristics of ILD are scarring (pulmonary fibrosis) and/or inflammation of the lungs. The interstitium is an interconnected fine mesh of tissue that extends through each lung, supporting the alveoli (air sacs) of the lung. Under normal conditions, the interstitium is so thin that it doesn't show up on X-rays or CT scans. All forms of ILD result in thickening of the interstitium, e.g., through inflammation, scarring, or a buildup of fluid. There is no universally accepted single classification of ILDs. They can generally be categorized based on their etiology (idiopathic or ILDs with known association or cause), clinical course (acute (transient), subacute or chronic (long-term) ILDs), and based on the main pathological features (inflammatory or fibrotic ILDs. A plethora of substances and conditions can lead to ILD. Even so, in some cases, the causes are never found. Such disorders without a known cause are grouped together under the label of idiopathic interstitial pneumonias, the most common and deadly of which is idiopathic pulmonary fibrosis (IPF).

During the progression of ILDs such as IPF, an accumulation of extra cellular matrix components such as collagen and an increase in the fibroblast population is observed. Persistent proliferation of fibroblasts is considered an important contributor to the lung architecture in IPF, including the diminished interstitial spaces of the alveoli. Thus, reducing TGF-β-induced proliferation of fibroblasts and structural collagen with LYT-100 has the potential to prolong lung function in IPF. In addition to inhibiting TGF-β-induced insoluble collagen level, LYT-100 also inhibits TGF-β-induced secreted collagen and fibronectin 3. Secreted collagen and fibronectin not only increase the rate of formation of fibrotic foci in the lung, these proteins can also act as ligands for integrin receptors. When integrin receptors are activated, they induce not only the proliferation of epithelial cells and fibroblasts of the lungs, but they also, along with TGF-β, induce epithelial mesenchymal transition (EMT) of the epithelial cells of the lungs. EMT causes these cells to migrate to different regions of the lungs. This migration is considered to be a very important contributor for the generation of new fibrotic foci in the lungs and progression of IPF. LYT-100 has the ability to inhibit TGF-β-induced pro-fibrotic processes and to reduce basal factors, which have the potential to exacerbate ongoing fibrosis.

Progressive fibrotic ILDs can divided into 3 groups based on their disease behavior and include intrinsically non-progressive, e.g. drug-induced lung disease after removal of the drug or some cases of hypersensitivity pneumonitis (HP) after removal of a trigger, progressive but stabilized by immunomodulation, e.g. some cases of connective tissue disease (CTD)-ILDs), and progressive despite treatment considered appropriate in individual ILDs, e.g. idiopathic pulmonary fibrosis (IPF).

While IPF is the best-known and prototypical form of a progressive fibrosing ILD (PF-ILD), there is a group of patients with different clinical ILD diagnoses other than IPF who develop a progressive fibrosing phenotype during the course of their disease. These patients demonstrate a number of similarities to patients with IPF, with their disease being defined by increasing extent of pulmonary fibrosis on imaging, declining lung function, worsening respiratory symptoms and quality of life despite treatment in individual ILDs, and, ultimately, early mortality Similar to IPF, a decline in FVC is predictive of mortality in patients with these other fibrosing ILDs.

Non-limiting examples of ILDs include non-idiopathic pulmonary fibrosis, idiopathic non-specific interstitial pneumonia (iNSIP), autoimmune or connective tissue disease (CTD)-ILDs, unclassifiable ILDs (uILD), chronic hypersensitivity pneumonitis (HP), interstitial pneumonia with autoimmune features (IPAF), genetic and/or familial idiopathic pulmonary fibrosis (g/f IPF), chronic sarcoidosis, exposure-related ILDs, and drug-induced ILDs.

In some embodiments, the ILD is iNSIP or interstitial pneumonia with autoimmune features (IPAF). In some embodiments, the ILD is chronic HP. Chronic HP is a complex syndrome caused by sensitization to an inhaled antigen that leads to an aberrant immune response in the small airways and lung parenchyma. Susceptibility is believed to be affected by genetics, antigen concentration and frequency of exposure, and immune tolerance.

In some embodiments, the ILD is autoimmune or CTD-ILD. Autoimmune diseases are commonly associated with pulmonary complications including ILD. Patients across the spectrum of CTDs are at risk of developing ILD. In some embodiments, the autoimmune or CTD-ILD is systemic sclerosis ILD (SSc-ILD). In some embodiments, the ILD is rheumatoid arthritis ILD (RA-ILD). In some embodiments, the ILD is lupus-induced pulmonary fibrosis. In some embodiments, the ILD is scleroderma interstitial lung disease. In some embodiments, the ILD is mixed CTD-associated ILD.

In some embodiments, the ILD is a childhood interstitial lung disease (chILD), which is a broad term for a group of rare lung diseases that can affect babies, children, and teens. These diseases have some similar symptoms, such as chronic cough, rapid breathing, and shortness of breath. These diseases also harm the lungs in similar ways. For example, they damage the tissues that surround the lungs' alveoli and bronchial tubes, and sometimes directly damage the air sacs and airways. The various types of chILD can decrease lung function, reduce blood oxygen levels, and disturb the breathing process. In some embodiments, the chILD is selected from a surfactant dysfunction mutation, a childhood lung developmental disorder such as alveolar capillary dysplasia, a lung growth abnormality, neuroendocrine cell hyperplasia of infancy (NEHI), pulmonary interstitial glycogenosis (PIG), idiopathic interstitial pneumonia (such as nonspecific interstitial pneumonia, cryptogenic organizing pneumonia, acute interstitial pneumonia, desquamative interstitial pneumonia, lymphocytic interstitial pneumonia), an alveolar hemorrhage syndrome, an aspiration syndrome, a hypersensitivity pneumonitis, an infectious or post infectious disease (bronchiolitis obliterans), eosinophilic pneumonia, pulmonary alveolar proteinosis, pulmonary infiltrates with eosinophilia, pulmonary lymphatic disorders (lymphangiomatosis, lymphangiectasis), pulmonary vascular disorders (haemangiomatosis), an interstitial lung disease associated with systemic disease process (such as connective tissue diseases, histiocytosis, malignancy-related lung disease, sarcoidosis, storage diseases), or a disorder of the compromised immune system (such as opportunistic infection, disorders related to therapeutic intervention, lung and bone marrow transplant-associated lung diseases, diffuse alveolar damage of unknown cause).

In some embodiments, the ILD is chronic sarcoidosis or sarcoidosis-related pulmonary fibrosis. Sarcoidosis is an inflammatory disease characterized by the formation of granulomas in one or more organs of the body. When left unchecked, this chronic inflammation can lead to fibrosis. Sarcoidosis affects the lungs in approximately 90% of cases but can affect almost any organ in the body.

In some embodiments, the method treats an ILD which is an exposure-related ILD, or a drug-induced ILD. In some embodiments, the exposure related ILD is pneumoconiosis. Pneumoconiosis is one of a group of ILDs caused by breathing in certain kinds of dust particles, such as asbestos, coal, or silica. In some embodiments, the exposure-related ILD is asbestos-induced pulmonary fibrosis, silica-induced pulmonary fibrosis, coal-induced pulmonary fibrosis, or other environmentally induced pulmonary fibroses.

In some embodiments, the ILD is acute interstitial pneumonia (AIP, also known as Hamman-Rich syndrome). AIP is an acute, rapidly progressive idiopathic pulmonary disease that often leads to fulminant respiratory failure and acute respiratory distress syndrome (ARDS). In some embodiments, the ILD is alveolitis, including, chronic fibrosing alveolitis and fibrosing alveolitis.

In some embodiments, the ILD is an unclassifiable ILD (uILD). The term “unclassifiable interstitial lung disease” was introduced in the American Thoracic Society/European Respiratory Society Consensus Classification of the Idiopathic Interstitial Pneumonias (IIP) in 2002 to encompass a subset of ILDs that cannot be classified within the confines of the current diagnostic framework. The paradoxical classification as “unclassifiable” results from either 1) inadequate or 2) discordant clinical, radiologic, and pathologic data, such that a specific ILD diagnosis is not possible.

Many ILDs are characterized by inflammation and chronic fibrosis. Patients with certain types of chronic fibrosing ILD are at risk of developing a progressive phenotype. These include, but are not limited to, iNSIP, uILD, autoimmune ILDs, chronic sarcoidosis, HP, g/f IPF, and exposure-related diseases, such as asbestosis and silicosis.

Because these various conditions share similarities regarding pathogenesis and clinical behavior, they are increasingly described under the umbrella terminology of “progressive fibrosing ILDs” (PF-ILDs) or “fibrosing ILD with a progressive phenotype.” A progressive phenotype is characterized histologically by self-sustaining fibrosis, a process common to a variety of conditions, and which leads to worsening quality of life, decline in lung function and, eventually, early mortality. The term “progressive phenotype” implies that progression of disease has occurred despite state-of-the-art management, including, for example, the use of corticosteroids and/or immunosuppressive therapy. One of the most common types of progressive fibrosing ILD is idiopathic pulmonary fibrosis (IPF). IPF is, by definition, a progressive fibrosing ILD of unknown cause, characterized by a decline in lung function and early mortality. The prognosis of IPF is poor, with the median survival after diagnosis generally estimated at 2 to 5 years.

In addition to IPF, PF-ILDs also include non-IPF ILDs. Estimates based on a survey and insurance claims in the USA indicate that 18-32% of patients diagnosed with non-IPF ILDs would develop progressive fibrosis (Wijsenbeek et al. “Progressive fibrosing interstitial lung diseases: current practice in diagnosis and management” Curr Med Res Opin 2019: 1-10). In the same study, time from symptom onset to death was estimated to be 61-80 months, a poor survival rate, yet better than that for IPF. The incidence and prevalence of PF-ILDs are not well defined, partly due to the heterogeneous nature of this group. It is estimated that there are on the order of 140,000-250,000 people in the United States living with PF-ILDs, including IPF. Currently, no drugs are approved for the treatment of progressive fibrotic ILDs other than nintedanib and pirfenidone for the treatment of IPF. Accordingly, LYT-100, having the potential for higher dosing than pirfenidone by virtue of its enhanced tolerability, may be particularly advantageous in treating PF-ILDs.

In some embodiments, the ILD is a PF-ILD. In some embodiments, the PF-ILD is iNSIP, a CTD-ILD, a uILD, chronic fibrotic HP, a g/f IPF, sarcoidosis, an exposure-related ILD, or a drug-induced ILD.

In some embodiments, the fibrotic-mediated pulmonary disease or disorder is not idiopathic pulonary fibrosis (IPF).

In treating any of the interstitial lung disease and other fibrotic-mediated pulmonary diseases or disorders disclosed herein, the LYT-100 is administered as disclosed herein above. In some embodiments, the LYT-100 is administered in a total daily dose from about 825 to about 2475 mg, such as 825 mg, 1650 mg or 2475 mg. In some embodiments, the administration is in three equal admninistrations. In some embodiments, the LYT-100 is administered in three equal doses of 550 mg each. In some embodiments, the LYT-100 is administered in three equal doses of 825 mg each.

### Clinical Endpoints and Biomarkers

In some embodiments, treatment efficacy may be evaluated by reference to various clinical endpoints or biomarkers indicative of fibrotic and inflammatory processes. Many of these clinical endpoints are described above with respect to the specific disease or disorder.

Suitable types of biomarkers include, but are not limited to, markers of alveolar epithelial cell injury and epithelial cell dysfunction, markers of alveolar macrophage activation, markers of TGF-β activation, markers of fibroblast proliferation and extracellular matrix production or turnover, markers of immune dysregulation, an markers of ECM production and turnover. In some embodiments, the biomarker is Krebs von den Lungen-6 antigen (KL-6), a surfactant protein (e.g., SP-A or SP-D), a matrix metalloprotease (e.g., MMP-1, MMP-7, MMP-8), PP1, YKL-40, IGFBP-1, TNFRSA1F, ICAM-1, IL-6, IL-8, a CC chemokine ligand (e.g., CCL 16 and CCL 18), Insulin-like growth factor (IGF), an IGF-binding protein (IGFBP), Vascular endothelial growth factor (VEGF), periostin, or a combination thereof.

In any of the methods described herein, the method of treating prevents, delays, or slows the progression of impaired respiratory function in the subject. In some embodiments, progression of ILD is delayed, slowed or arrested.

Respiratory function, e.g., impaired respiratory function, can be measured using various methods. In some embodiments, the respiratory function is determined by measuring Forced Vital Capacity (FVC) in the subject. In some embodiments, the progression of impaired respiratory function in the subject is determined by measuring a change in FVC over a period of treatment.

In some embodiments, the change in FVC is measured as a rate of decline in FVC (mL). In one aspect is provided a method of treating ILD, the method comprising administering to a subject in need thereof a total daily dose of 825 mg administered in three equal doses of 275 mg each of LYT-100, wherein the the rate of decline in FVC (mL) is lower relative to a subject who has not received LYT-100. In one aspect is provided a method of treating ILD, the method comprising administering to a subject in need thereof a total daily dose of 1650 mg administered in three equal doses of 550 mg each of LYT-100, wherein the the rate of decline in FVC (mL) is lower relative to a subject who has not received LYT-100. In one aspect is provided a method of treating ILD, the method comprising administering to a subject in need thereof a total daily dose of 2475 mg administered in three equal doses of 825 mg each of LYT-100, wherein the the rate of decline in FVC (mL) is lower relative to a subject who has not received LYT-100. In some embodiments, the period of treatment for measuring the rate of decline in FVC (mL) is at least 26 weeks. In some embodiments, the period of treatment for measuring the rate of decline in FVC (mL) is at least 52 weeks. In some embodiments, the rate of decline in FVC (mL) over at least a 26-week treatment period is a value less than the rate of decline exhibited by a subject who has not received LYT-100. In some embodiments, the rate of decline in FVC (mL) over at least a 52-week treatment period is a value less than the rate of decline exhibited by a subject who has not received LYT-100.

In some embodiments, the change in FVC is measured as a change in FVC % predicted (FVCpp). In some embodiments, the change in FVC is measured as a decline in FVC % predicted (FVCpp). In one aspect is provided a method of treating ILD, the method comprising administering to a subject in need thereof a total daily dose of 825 mg administered in three equal doses of 275 mg each of LYT-100, wherein the the rate of decline in FVCpp is lower relative to a subject who has not received LYT-100. In one aspect is provided a method of treating ILD, the method comprising administering to a subject in need thereof a total daily dose of 1650 mg administered in three equal doses of 550 mg each of LYT-100, wherein the the rate of decline in FVCpp is lower relative to a subject who has not received LYT-100. In one aspect is provided a method of treating ILD, the method comprising administering to a subject in need thereof a total daily dose of 2475 mg administered in three equal doses of 825 mg each of LYT-100, wherein the the rate of decline in FVCpp is lower relative to a subject who has not received LYT-100.

In some embodiments, the treatment of ILD is demonstrated or exhibited by a delay in the time to progression of ILD in the subject. In some embodiments, the treatment of ILD is demonstrated or exhibited by a slower rate of progression of ILD in the subject. In any of the methods disclosed herein, the length of time to ILD progression is longer (increased, greater) in the subject treated with LYT-100 relative to a subject who has not received LYT-100. ILD progression can be determined using various methods, including by measuring the change in FVC, e.g., a decline in FVC mL or FVCpp. In some embodiments, IPF progression is determined by a decline in FVCpp of 5% or greater. In some embodiments, IPF progression is determined by a decline in FVCpp of 10% or greater. In any of the methods disclosed herein, the length of time to ILD progression, as determined by a decline in FVCpp of 5% or greater, is longer (increased, greater) in the subject treated with LYT-100 relative to a subject who has not received LYT-100. In any of the methods disclosed herein, the length of time to ILD progression, as determined by a decline in FVCpp of 10% or greater, is longer (increased, greater) in the subject treated with LYT-100 relative to a subject who has not received LYT-100.

In any of the methods disclosed herein, the subject exhibits a longer period of time to hospitalization due to impaired respiratory function relative to a subject who has not received LYT-100. In some instances, the longer length of time to hospitalization is a longer length of time for an initial hospitalization due to impaired respiratory function. In some instances, the longer lengthof time to hospitalization is not an initial hospitalization, e.g., it is a longer length of time for subsequent hospitalization(s) due to impaired respiratory function.

In any of the methods disclosed herein, the subject has less frequent hospitalizations due to impaired respiratory function relative to a subject who has not received LYT-100. Thus, in some embodiments, the subject has a lower number of hospitalizations due to impaired respiratory function relative to a subject who has not received LYT-100. In any of the methods disclosed herein, the subject has a shorter duration of hospitalization time(s) due to impaired respiratory function relative to a subject who has not received LYT-100.

In any of the methods disclosed herein, the subject exhibits a longer period of time to mortality due to impaired respiratory function relative to a subject who has not received LYT-100. In any of the methods disclosed herein, the subject exhibits a longer period of time to mortality due to IPF relative to a subject who has not received LYT-100.

In any of the methods disclosed herein, the subject has a change in one or more serum biomarker(s) related to impaired respiratory function relative to a subject who has not received LYT-100. In some embodiments, the serum biomarker is collagen type 4.

In any of the methods disclosed herein, the subject is treated as determined by one or more of: King's Briefinterstitial Lung Disease Questionnaire (K-BILD) total score; Saint George Respiratory Questionnaire (SGRQ-I) domain score; EuroQol 5-Dimensional (EQ5D) Questionnaire score; and Cough visual analog scale (VAS), relative to a subject who has not received LYT-100.

In any of the methods disclosed herein, the subject is treated without any dose reduction in the administered daily dose over the course of treatment. In any of the methods disclosed herein, the subject is treated without any interruption in treatment or temporary stoppage in treatment over the course of treatment. In any of the methods disclosed herein, the subject is treated without any discontinuation in treatment over the course of treatment.

In one aspect is provided a method for reducing the number of one or more adverse event(s) (AE) in the treatment of ILD, the method comprising administering to a subject in need thereof a total daily dose of 825 mg administered in three equal doses of 275 mg each of LYT-100. In one aspect is provided a method for reducing the number of one or more adverse event(s) (AE) in the treatment of ILD, the method comprising administering to a subject in need thereof a total daily dose of 1650 mg administered in three equal doses of 550 mg each of LYT-100. In one aspect is provided a method for reducing the number of one or more adverse event(s) (AE) in the treatment of ILD, the method comprising administering to a subject in need thereof a total daily dose of 2475 mg administered in three equal doses of 825 mg each of LYT-100.

Any of the above-described methods, the one or more adverse event(s) is a gastrointestinal-related adverse event selected from nausea, vomiting, abdominal pain or distension, dyspepsia, diarrhea, decreased appetite, and constipation. In any of the above-described methods, the one or more adverse event(s) is a nervous system-related adverse event selected from headache, dizziness, and somnolence. In any of the above-described methods, the one or more adverse event(s) is selected from fatigue, drug intolerance, and photosensitivity. In any of the above-described methods, the one or more adverse event(s) is selected from increased AST, ALT, GGT, and liver toxicity.

### Pharmaceutical Compositions

In another aspect, pharmaceutical compositions are provided for administration in the methods described herein. Pharmaceutical compositions include the active compound, e.g., LYT-100, and one or more pharmaceutically acceptable excipients or carriers. Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Embodiments of the present disclosure can be further defined by reference to the following non-limiting examples. It will be apparent to those skilled in the art that many modifications, both to materials and methods, can be practiced without departing from the scope of the present disclosure.

### EXEMPLIFICATION

Examples 1 and 2 provide crossover studies comparing the safety, tolerability, and pharmacokinetics of deupirfenidone (LYT-100) and pirfenidone. Example 3 provides a study exploring tolerability of the deuterated pirfenidone LYT-100 in patients with COVID-19 Respiratory Illness. Example 4 provides the CYP isozyme profile of pirfenidone and LYT-100. Example 5 provides a BioMAP Fibrosis Panel screening study for LYT-100 and pirfenidone across a series of fibrosis biomarkers. Example 6 provides a rat lipopolysaccharide (LPS) model of systemic inflammation. Example 7 provides a streptozocin-induced non-alcoholic steatohepatitis (NASH) mouse model. Example 8 provides a study of inhibition of fibrosis with LYT-100 in Primary Mouse Lung Fibroblasts. Example 9 provides a study of inhibition of collagen synthesis with LYT-100. Example 10 provides a mouse model of lympiedema. Example 11 provides a rat bleomycin-induced pulmonary fibrosis model.

### Example 1: Crossover Dosing Study

This study was a double-blind, randomized, two-period crossover study in older, healthy subjects to compare the safety, tolerability, and pharmacokinetics of deupirfenidone (LYT-100) and pirfenidone. The crossover study was performed at a single Study Center per Part in the United States.

### Study Description

This study was conducted in two Parts: 1 and 2.

Part 1 was a randomized, double-blinded, two period crossover study conducted in healthy older adults to compare the safety, tolerability, and pharmacokinetics of deupirfenidone (LYT-100) with twice daily (BID) dosing of LYT-100 to pirfenidone.

Part 2 was a randomized, double-blinded, two period crossover study conducted in healthy older adults to compare the safety, tolerability, and pharmacokinetics of deupirfenidone (LYT-100) with three times daily (TID) dosing of LYT-100 to pirfenidone.

### Study Endpoints

### Safety:

Treatment-emergent adverse events (TEAEs), including severity, and relatedness to study drug)
Physical examination
Vital signs
Electrocardiograms (ECGs)
Clinical laboratory parameters, including hematology, serum chemistry, coagulation, and urinalysis
New-onset concomitant medications

### Pharmacokinetics:

Comparison of the key PK parameters (C_{max,ss}, C_{min,ss}, and AUC_{0-24,ss}) between the parent compound (LYT-100 and pirfenidone) and primary metabolite (5-carboxypirfenidone). Other PK parameters were derived and compared.
Comparison of the key urine PK parameters (Aeₜ₁₋ₜ₂, CL_{R}, Feₜ₁₋ₜ₂) between the parent compound (LYT-100 and pirfenidone) and primary metabolite (5-carboxypirfenidone). Other urine PK parameters were derived and compared.
Food effect evaluation of LYT-100 and pirfenidone (C_{max,ss}, and AUC_{0-6,ss}) for fed versus fasted.

### Study Design

### Part 1:

Part 1 was a double-blind, randomized, two-period crossover study conducted in older, healthy subjects to determine the safety, tolerability, and PK of LYT-100 administered twice daily (BID) for 3 days (to steady state [Day 1 to Day 3 and Day 11 to Day 13]) compared to pirfenidone administered 3 times daily (TID) for 3 days (to steady state) under fed conditions. A final single dose of study drug (LYT 100 or pirfenidone) was administered on the morning of the fourth day in each treatment period (Day 4/Day 14) following an overnight fast of at least 8 hours to determine the effect of food on steady state PK parameters.

Over encapsulation was utilized to match TID dosing for pirfenidone and to match the number of LYT-100 capsules administered for each dose. Thus, during LYT-100 treatment, the mid-day dose was placebo. Pirfenidone was administered at the current marketed dose of 801 mg TID (2403 mg daily dose).

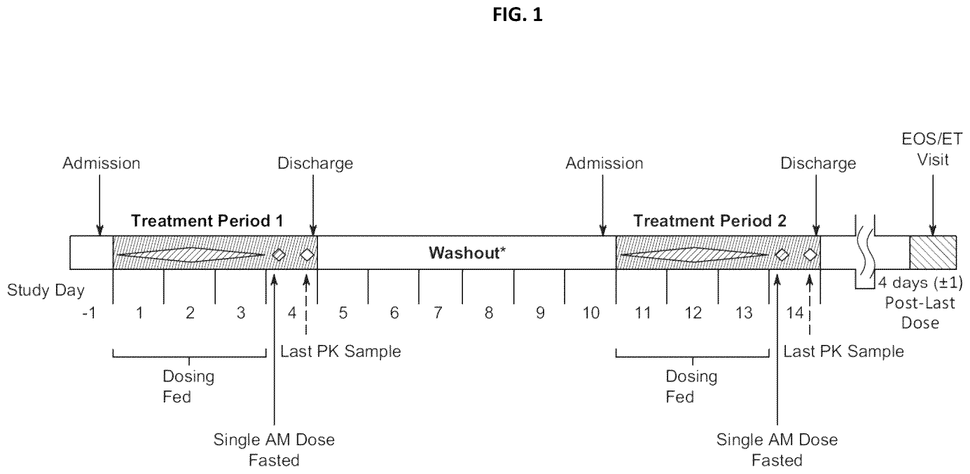

Approximately forty older healthy female and male adult subjects (1:1 target ratio) were randomized into 1 of 2 cohorts: Cohort 1 or Cohort 2, N=20 subjects per cohort; minimum of 8 per sex per cohort. Subjects in each cohort were randomized to treatment sequence as follows: Sequence A: Pirfenidone to LYT-100; Sequence B: LYT-100 to pirfenidone. Dosing is outlined in Table 8. A graphical illustration of the study design for Part 1 is provided as FIG. 1.

TABLE 8
Dosing Regimens by Cohort and Treatment Sequence (Part 1)

| | Treatment | Treatment Period 1 | | Treatment Period 2 | |
|---|---|---|---|---|---|
| Cohort | Sequent | Days 1 to 3 | Day 4 | Days 11 to 13 | Day 14 |
| 1 | A (n = 10) | Pirfenidone | Pirfenidone | LYT-100 | LYT-100 |
| | | 801 mg TID | 801 mg (AM | up to 1500 mg | up to |
| | | (every 6 hours) | only) | BID (every | 1500 mg*(AM |
| | | Fed | Fasted | 12 hours) + | only) |
| | | | | placebo at | Fasted |
| | | | | mid-day^{#} | |
| | | | | Fed | |
| | B (n = 10) | LYT-100 | LYT-100 | Pirfenidone | Pirfenidone |
| | | up to 1500 mg | up to 1500 mg | 801 mg TID | 801 mg (AM |
| | | BID (every | (AM only) | (every 6 hours)\hat{ } | only) |
| | | 12 hours) + | Fasted | Fed | Fasted |
| | | placebo at | | | |
| | | mid-day^{#} | | | |
| | | Fed | | | |
| 2 | A (n = 10) | Pirfenidone | Pirfenidone | LYT-100 | LYT-100 |
| | | 801 mg TID | 801 mg (AM | up to 1500 mg | up to 1500 mg |
| | | (every 6 hours) | only) | BID (every | (AM only) |
| | | Fed | Fasted | 12 hours) + | Fasted |
| | | | | placebo at | |
| | | | | mid-day^{#} | |
| | | | | Fed | |
| | B (n = 10) | LYT-100 | LYT-100 | Pirfenidone | Pirfenidone |
| | | up to 1500 mg | up to 1500 mg | 801 mg TID | 801 mg (AM |
| | | BID (every | (AM only) | (every 6 hours) | only) |
| | | 12 hours) + | Fasted | Fed | Fasted |
| | | placebo at mid-day | | | |
| | | Fed | | | |

The initial dose of LYT-100 for this crossover study directly comparing LYT-100 to pirfenidone in healthy adults was 850 mg BID LYT-100 (1700 mg daily dose) vs. 801 mg TID pirfenidone (2403 mg daily dose). The 850 mg BID LYT-100 (1700 mg daily dose) was selected based on the PK results from earlier studies. PK modelling work using data from the multiple ascending dose study and a single-dose crossover study of LYT-100 and pirfenidone indicated that a dose of LYT-100 of approximately 800-850 mg BID (1600-1700 mg daily dose) results in a similar systemic exposure to the marketed dose of pirfenidone (2403 mg daily dose). Based on these data, a randomized blinded cross-over study in older healthy adults was conducted (N=37) administering LYT-100 850 mg BID 3 days fed dosing versus pirfenidone 801 mg TID 3 days fed dosing. The study is blinded with a placebo mid-day dose for LYT-100 to match TID pirfenidone dosing. There was a single AM fasting dose on Day 4 for both drugs. There was a 6-day wash-out period between drug cross-over. The 850 mg BID dose was selected as a match to the exposure for pirfenidone based on the outcome of the earlier PK crossover study, which indicated that an 850 mg BID daily dose of LYT-100 has about 102% of the steady-state systemic exposure of pirfenidone dosed daily at 801 mg TID.

### Part 2:

Part 2 was a double-blind, randomized, two-period crossover study conducted in older healthy subjects to determine the safety, tolerability, and PK of LYT-100 administered three times daily (TID) for 3 days (to steady state [Day 1 to Day 3 and Day 11 to Day 13]) compared to pirfenidone administered TID for 3 days (to steady state) under fed conditions. A final single dose of study drug (LYT-100 or pirfenidone) was administered on the morning of the fourth day in each treatment period (Day 4/Day 14) following an overnight fast of at least 8 hours to determine the effect of food on steady state PK parameters. Over-encapsulation was utilized to maintain study blind. Screening was performed up to 28 days prior to administration of the first dose of LYT-100/pirfenidone. Only subjects who met all the applicable inclusion and none of the applicable exclusion criteria were randomized. Approximately 50 older healthy female and male adult subjects (1:1 ratio) were randomized into 1 of 2 cohorts: Cohort 1 or Cohort 2, N=˜25 subjects per cohort. Subjects in each cohort were randomized to treatment sequence as follows:
Sequence A: Pirfenidone to LYT-100
Sequence B: LYT-100 to pirfenidone

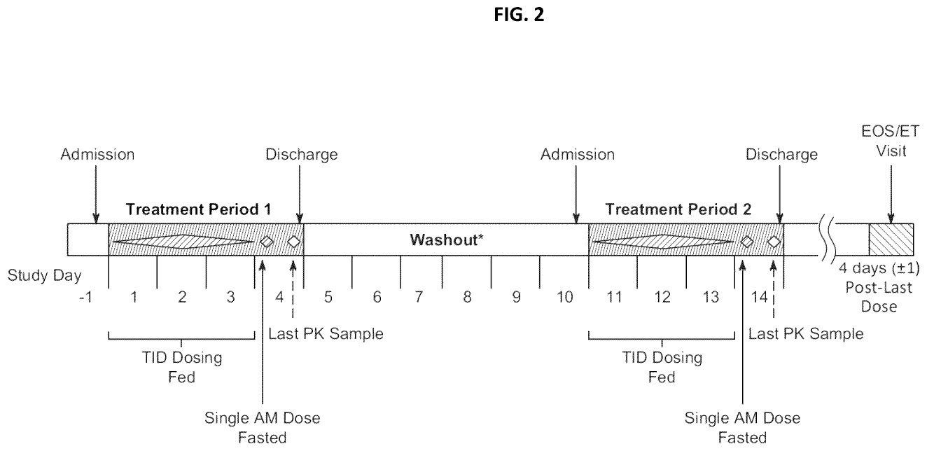

A graphical illustration of the study design for Part 2 is provided as FIG. 2. Dosing is outlined in Table 9.

TABLE 9
Dosing Regimens by Cohort and Treatment Sequence (Part 2)

| | Treatment | Treatment Period 1 | | Treatment Period 2 | |
|---|---|---|---|---|---|
| Cohort | Sequent | Days 1 to 3 | Day 4 | Days 11 to 13 | Day 14 |
| 1 | A | Pirfenidone | Pirfenidone | LYT-100, 550 | LYT-100 550 mg |
| (n = 25) | | 801 mg TID | 801 mg (AM | mg TID (every 6 | single dose (AM |
| | | (every 6 hours) | only) | hours), Fed | only), Fasted |
| | | Fed | Fasted | | |
| | B | LYT-100, 550 mg | LYT-100 550 | Pirfenidone | Pirfenidone |
| | | TID (every 6 | mg single dose | 801 mg TID | 801 mg (AM |
| | | hours), Fed | (AM only), | (every 6 hours)\hat{ } | only) |
| | | | Fasted | Fed | Fasted |
| 2 | A | Pirfenidone | Pirfenidone | LYT-100, 550 | LYT-100 550 mg |
| (n = 25) | | 801 mg TID | 801 mg (AM | mg TID (every 6 | single dose (AM |
| | | (every 6 hours) | only) | hours), Fed | only), Fasted |
| | | Fed | Fasted | | |
| | B | LYT-100, 550 mg | LYT-100 550 | Pirfenidone | Pirfenidone |
| | | TID (every 6 | mg single dose | 801 mg TID | 801 mg (AM |
| | | hours), Fed | (AM only), | (every 6 hours) | only) |
| | | | Fasted | Fed | Fasted |

\hat{ }Pirfenidone: AM dose: 3 × 267 mg, mid-day dose: 3 × 267 mg, PM dose: 3 × 267 mg. Each of the AM, mid-day, and PM doses are over-encapsulated to maintain study blind. Placebo capsules are administered as needed to match the number of LYT-100 capsules in order to maintain the blind. Each cohort starting concurrently or closely staggered.

The LYT-100 dose for this crossover study directly comparing LYT-100 to pirfenidone in healthy adults was 550 mg TID LYT-100 (1650 mg daily dose) vs. 801 mg TID pirfenidone (2403 mg daily dose). The 550 mg TID LYT-100 (1650 mg daily dose) was selected based on the PK results from earlier studies and the PK results obtained in Part 1 of this study. PK modelling work using data from the multiple ascending dose study, the single-dose crossover study of LYT-100 and pirfenidone and Part 1 of this study indicated that a dose of LYT-100 550 mg TID (1650 mg daily dose) results in a similar systemic exposure to the marketed dose of pirfenidone (2403 mg daily dose). Particularly, it was predicted that a dose of 550 TID LYT-100 (1650 mg total daily dose) would achieve a steady-state systemic exposure that is about 99% of the steady-state systemic exposure observed for pirfenidone dosed at 801 mg TID.

Based on these data, a randomized blinded cross-over study in older healthy adults was conducted (N=49) administering LYT-100 550 mg TID 3 days fed dosing versus pirfenidone 801 mg TID 3 days fed dosing. There was a single AM fasting dose on Day 4 for both drugs. There was a 6-day wash-out period between drug cross-over. The 550 mg TID dose was selected as a match to the exposure for pirfenidone based on the outcome of the earlier PK crossover studies.

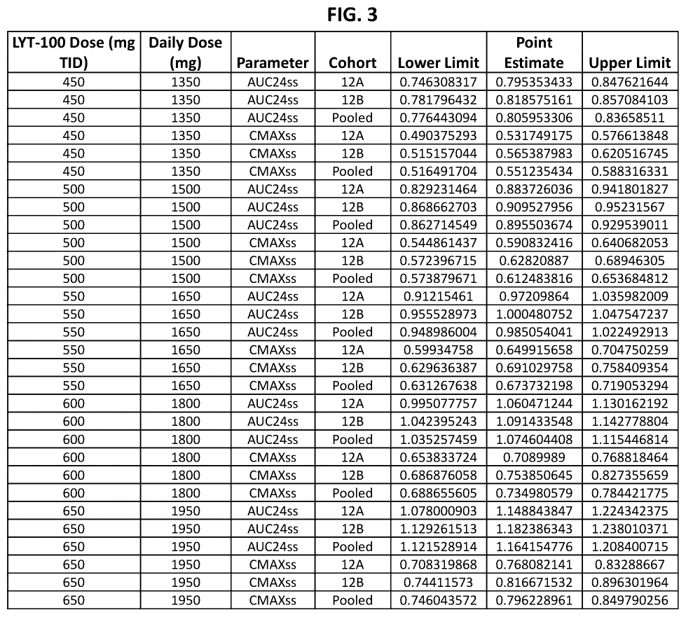

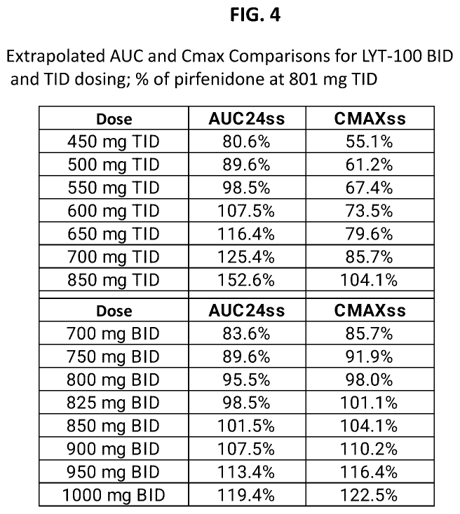

See FIG. 3 and FIG. 4, which show that the predicted steady-state systemic exposure (AUC24ss) for LYT-100 dosed at 550 TID is 98.5% of the steady-state systemic exposure (AUC24ss) of pirfenidone dosed at 801 mg TID. Surprisingly, however, the Cₘₐₓ for LYT-100 dosed at 550 mg TID is predicted to be lower than the pirfenidone Cₘₐₓ resulting from pirfenidone administered at 801 mg TID. FIG. 4 shows that the predicted steady-state Cₘₐₓ for LYT-100 dosed at 550 mg TID is 67.4% of the steady-state Cₘₐₓ for pirfenidone dosed at 801 mg TID. Without wishing to be bound by any particular theory, it is believed that the lower Cₘₐₓ of LYT-100 may contribute to the enhanced tolerability of LYT-100 relative to pirfenidone.

### Treatment Period 1 (Day −1 to Day 4) Parts 1 and 2

Subjects were admitted to the Clinical Research Unit (CRU) on Day −1 of Treatment Period 1 and were administered their assigned study drug (pirfenidone or LYT-100, with or without matching placebo) every 6 hours for 3 days until steady state (Day 1 to Day 3) under fed conditions. Subjects were then administered a single dose of their randomized treatment (pirfenidone or LYT-100, with or without matching placebo) on the morning of Day 4 following an overnight fast of at least 8 hours. Subjects were discharged on Day 4 following successful completion of all assessments and at the Investigator's discretion.

### Treatment Period 2 (Day 11 to Day 14) Parts 1 and 2

Following a minimum washout period of at least 7 days, subjects returned to the CRU and were admitted on the evening of Day 10 and were crossed over and administered the alternate study drug (pirfenidone or LYT-100, with or without matching placebo) every 6 hours for 3 days (Day 11 to Day 13) under fed conditions. Subjects were then administered a single dose of their randomized treatment on the morning of Day 14 following an overnight fast of at least 8 hours. Subjects were discharged on Day 14 following successful completion of all assessments and at the Investigator's discretion.

On Days 1 to 3 (Treatment Period 1) and Days 11 to 13 (Treatment Period 2) subjects were administered their assigned study drug TID, every 6 hours±0.25 hours (with approximately 240 mL of non-carbonated water), 30 minutes after the start of consumption of their standardized breakfast, lunch, or dinner (6 hours apart). An evening snack was served ≥3 hours following evening study medication administration. On Day 4 (Treatment Period 1) and Day 14 (Treatment Period 2), subjects were administered their assigned study drug once in the morning following an overnight fast of at least 8 hours (with approximately 240 mL of non-carbonated water). No additional fluids were allowed during the 1 hour pre- and post-dose

On Fed Days, meals were provided as follows:
Breakfast: meal served 30 mins prior to AM dosing. Breakfast was completed within 30 mins of start time.
Lunch: meal served at least 4 h post-AM study drug dose, and 30 minutes prior to the mid-day dose in Part 5 (only).
Dinner: meal served at least 11.5 h post-AM dose and served 30 minutes prior to PM study drug dose.
Evening snack: Snack served at least 15 h post-AM dose (at least 3 h post-PM dose).

On Fasted Days, meals were provided as follows:
On Day 4 (Period 1) and Day 14 (Period 2), breakfast was provided ≥4 hours post-study drug administration.

### Number of Subjects:

### Part 1

The objective was to recruit approximately 40 healthy older female and male adult subjects (target ratio 1:1 of males: females with a minimum of 8 per sex per cohort), unless additional subjects were required to support the statistical analysis. Part 1 was conducted with N=37 subjects who completed the study.

### Part 2

The objective was to recruit approximately 50 healthy older female and male adult subjects (target ratio 1:1 of males: females with a minimum of 15 per sex per cohort), unless additional subjects were required to support the statistical analysis. Part 1 was conducted with N=49 subjects who completed the study.

### Main Criteria for Inclusion and Exclusion

### Inclusion Criteria:

1. Male or female between 60 and 80 years old (inclusive) at the time of screening.
2. Subjects have a body mass index (BMI) between ≥18.0 and ≤35.0 kg/m² at screening.
3. Willing and able to abstain from direct whole body sun exposure from 2 days prior to dosing and until final study procedures have been conducted. Subjects were instructed to avoid or minimize exposure to sunlight (including sunlamps), use an SPF 50 sun block, or higher, wear clothing that protects against sun exposure and avoid concomitant medications known to cause photosensitivity (including but not limited to tetracycline, doxycycline, nalidixic acid, voriconazole, amiodarone, hydrochlorothiazide, naproxen, piroxicam, chlorpromazine and thioridazine).

### Exclusion Criteria:

1. Pregnant or lactating at screening or baseline or planning to become pregnant (self or partner) at any time during the study, including the specified follow-up period.
2. History or presence of malignancy at screening or baseline, with the exception of adequately treated localised skin cancer (basal cell or squamous cell carcinoma) or carcinoma in-situ of the cervix.
3. Clinically significant infection within 28 days of the start of dosing, or infections requiring parenteral antibiotics within the 3 months prior to screening. Known exposure to another person with COVID-19 within the last 14 days is also an exclusion criterion, or a positive COVID test within five days prior to dosing.
4. Had major surgery, (e.g., requiring general anesthesia) within 3 months before Screening, based on Investigator's discretion or has surgery planned during the time the participant is expected to participate in the study.
5. Suffering from clinically significant systemic allergic disease at screening or baseline or has a history of significant drug allergies including a history of anaphylactic reaction (particularly reactions to general anaesthetic agents); allergic reaction due to any drug which led to significant morbidity; prior allergic reaction to pirfenidone.
6. Chronic administration (defined as more than 14 consecutive days) of immunosuppressants or other immune-modifying drugs within 3 months prior to study drug administration. Corticosteroids are permitted at the discretion of the Investigator.
7. History or presence at screening or baseline of a condition associated with significant immunosuppression.
8. Positive test for hepatitis C antibody (HCV), hepatitis B surface antigen (HBsAg), or human immunodeficiency virus (HIV) antibody at screening.
9. Symptoms of dysphagia at screening or baseline or known difficulty in swallowing capsules.
10. Any condition at screening or baseline (e.g., chronic diarrhoea, inflammatory bowel disease or prior surgery of the gastrointestinal tract) that would interfere with drug absorption or any disease or condition that is likely to affect drug metabolism or excretion, at the discretion of the Investigator.
11. History or presence at screening or baseline of cardiac arrhythmia or congenital long QT syndrome.
12. QT interval corrected using Fridericia's formula (QTcF) >450 msec. ECG may be repeated 30 to 60 minutes apart from the first one collected at screening. If repeat ECG is ≤450 msec, the second ECG may be used to determine patient eligibility. However, if repeat ECG confirms QTcF remains >450 msec, the subject is not eligible.
13. Use of tobacco or nicotine containing products in the previous 3 months prior to dosing or a positive urine cotinine test at Screening or Baseline.
14. Lack of willingness to abstain from the consumption of tobacco or nicotine-containing products throughout the duration of the study and until completion of the final Follow-up visit.
15. Regular alcohol consumption defined as >21 alcohol units per week (where 1 unit=284 mL of beer, 25 mL of 40% spirit or a 125 mL glass of wine) or the Participant is unwilling to abstain from alcohol for 48 h prior to admission and 48 h prior to study visits.
16. Use of any of the following drugs within 30 days or 5 half-lives of that drug, whichever is longer, prior to study drug administration:
   a. Fluvoxamine, enoxacin, ciprofloxacin;
   b. Other inhibitors of CYP1A2 (including but not limited to methoxsalen or mexiletine);
   c. Contraceptives containing oestradiol, ethinyloestradiol or gestodene;
   d. Inducers of CYP1A2 (such as phenytoin), CYP2C9 or 2C19 (including but not limited to carbamazepine or rifampin);
   e. Any drug associated with prolongation of the QTc interval (including but not limited to moxifloxacin, quinidine, procainamide, amiodarone, sotalol).
17. Vaccination with a live vaccine within the 4 weeks prior to screening or that is planned within 4 weeks of dosing, and any non-live vaccination within the 2 weeks prior to screening or that is planned within 2 weeks of dosing (including those for COVID).
18. Use of any investigational drug or device within the longer of 30 days or five half-lives prior to screening.
19. Consumption of grapefruit, grapefruit juice, Seville oranges, Seville orange juice, or any foods containing these ingredients, within 7 days prior to dosing or unwilling to abstain from these throughout the duration of the study.

### Dosage and Mode of Administration:

This was a crossover study in which subjects received both the test treatment (LYT-100) and the reference (pirfenidone). All subjects received LYT-100 (BID or TID) or pirfenidone (TID) for 3 days in each respective treatment period, with placebo over-encapsulation to maintain the blind. Part 1 subjects received LYT-100 850 mg BID. Part 2 subjects received LYT-100 550 mg TID. In Parts 1 and 2, all subjects also received a single dose of either LYT-100 or pirfenidone on the morning of the fourth day in each respective treatment period with placebo over-encapsulation to maintain the blind.
LYT-100 (Deupirfenidone) was provided as hard gelatin capsules. LYT-100 was stored at a controlled room temperature of 15° C. to 25° C.
Pirfenidone (Esbriet) was provided as white to off-white hard gelatin capsules contain 267 mg of pirfenidone.
Both LYT-100 and pirfenidone were over-encapsulated to maintain study blind.

### Duration of Treatment:

### Parts 1 and 2

This study included a 28-day Screening period, two treatment periods (each 4 days in duration) with a minimum 7-day washout period between treatment periods, and a 3-day (±1 day) post-last-dose safety follow-up visit. Thus, total duration of study participation for each subject was approximately 50 days. Treatment with double-blind study medication was 4 days for each of the two treatment periods, 8 days in total.

### Criteria for Evaluation

### Safety:

Safety and tolerability was assessed by monitoring AEs, physical examination, vital signs, 12-lead ECGs, clinical laboratory values (hematology panel, multiphasic chemistry panel and urinalysis), and review of concomitant treatments/medication use.

### Pharmacokinetics:

### Parts 1 and 2

Subjects provided blood samples prior to treatment, i.e., Day −1 or Day 1 in Treatment Period 1, for the determination of CYP1A2, CYP2C9, CYP2C19, and CYP2D6 genotype to support exploratory PK analyses. Subjects were required to provide consent for genotyping.

Blood samples for PK were collected for Cohorts 1 and 2 at specified times during both periods, as follows:
Days 1 & 11: 0 (pre-morning dose)
Days 2 & 12: no sampling
Days 3 & 13: 0 (pre-morning dose), and 1, 2, 3, 4, 6 (pre-mid-day dose), 7, 8, 9, 10, 12 (pre-evening dose), 13, 14, 15, 16, and 17 hours post-morning dose
Days 4 & 14: 0 (pre-morning dose), and 1, 2, 3, 4, 6 (post-dose),

Plasma PK parameters for steady state dosing (Days 1 to 3 and Days 11 to 13) included, but are not limited to:
AUC_{0-tau,ss} (area under the time concentration curve from time zero to tau at steady state)
AUC_{0-24,ss} (area under the time concentration curve from time zero to 24 hours at steady state)
λz (terminal disposition rate constant/terminal rate constant)
t½ (elimination half-life)
C_{max,ss} (maximum concentration in a dosing interval)
Tₘₐₓ (time to maximum concentration, as reported relative to the beginning of a dosing interval in which maximum concentration occurred)
C_{min,ss} (lowest concentration in a dosing interval)
C_{av,ss} (average concentration during a dosing interval)
C_{max,ss}−C_{min,ss}/C_{av,ss} (degree of fluctuation)
C_{max,ss}−C_{min,ss}/C_{min,ss} (swing)
PTF % (peak-trough fluctuation)

Plasma PK parameters for food effect analysis (Days 4 and 14) included, but are not limited to:
AUC_{0-tau,ss} (area under the time concentration curve from time zero to tau at steady state)
AUC_{0-6,ss} (area under the time concentration curve from time zero to 6 hours at steady state)
AUC_{0-∞} (area under the time concentration curve from time zero to infinity)+AUC_{0-∞}/D
% AUCₑₓₜ (area under the time concentration curve extrapolated from time t to infinity as a percentage of total AUC)
λz (terminal disposition rate constant/terminal rate constant)
CL/F (apparent total clearance)
Vz/F (apparent volume of distribution)
Tₘₐₓ (time to maximum concentration)
t_{lag} (lag time)

### Part 1 Only

Urine samples for PK were collected for Cohorts 1 and 2 at specified intervals during both treatment periods, as follows:
Days 1 and 11: pre-dose (subjects instructed to empty their bladders approximately 30 minutes prior to dosing)
Days 2 and 12: no urine sampling
Days 3 and 13: pre-dose (subjects instructed to empty their bladders approximately 30 minutes prior to dosing), 0 to 4, 4 to 8, 8 to 12, 12 to 16, and 16 to 24 hours post-AM dose
Days 4 and 14: 0 to 3 and 3 to 6 hours post-AM dose

Urine samples for analysis of excretion in urine were collected, separated by specified time interval, and analyzed. The total volume of urine collected in each interval (t1 to t2) was noted. The urine PK parameters included, but are not limited to:
Ae_{t1/2} (Amount excreted in urine over time)
CLR (Renal clearance)
Fraction of systemic clearance (CL/F) represented by the renal clearance (CLR/[CL/F])
Fet1-t2 (Fraction of administered dose excreted in urine over the dosing intervals)
   Study Endpoints were Defined as Follows:

### Safety

AEs (type, severity, and relatedness to study drug)
Physical examination
Vital signs
Electrocardiograms (ECGs)
Clinical laboratory parameters (hematology, serum chemistry, coagulation, and urinalysis)
New-onset concomitant medications

### Pharmacokinetics:

Comparison of the key plasma PK parameters (C_{max,ss}, C_{min,ss}, and AUC_{0-24,ss}) between the parent compound (LYT-100 and pirfenidone) and primary metabolite (5-carboxypirfenidone). Other plasma PK parameters will also be derived and compared.
Comparison of the key urine PK parameters (Aeₜ₁₋ₜ₂, CL_{R}, Feₜ₁₋ₜ₂) between the parent compound (LYT-100 and pirfenidone) and primary metabolite (5-carboxypirfenidone). Other urine PK parameters may be derived and compared.
Food effect evaluation of LYT-100 and pirfenidone (C_{max,ss}, and AUC_{0-6,ss}) for fed vs fasted.

### Results

### Part 1

It was determined that 1000 mg BID of LYT-100 provided an exposure (AUC) of LYT-100 which was greater than the exposure of pirfenidone resulting from administration of the approved dose of pirfenidone (801 mg TID). It was further determined based on dose projections that doses of LYT-100 in the range of 800 to 850 mg BID would provide exposure (AUC) and maximal concentration (Cₘₐₓ) values of LYT-100 which are comparable to those of pirfenidone when administered at 801 mg TID (2403 mg daily).

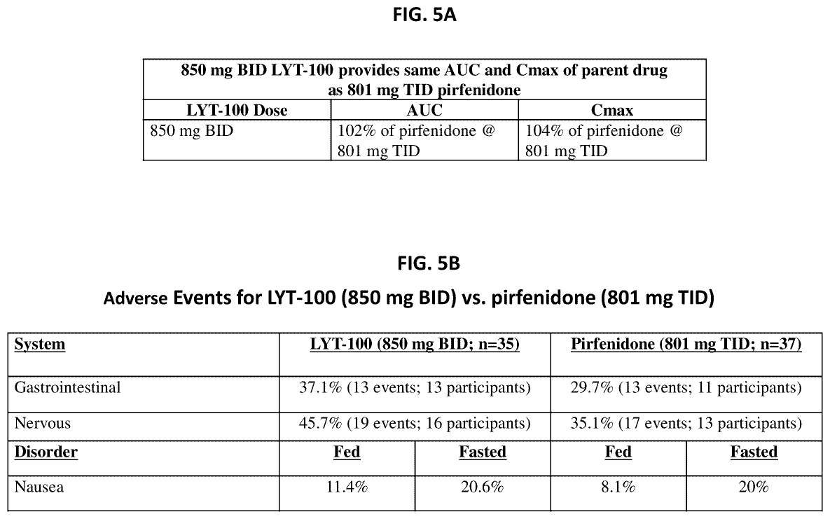

The Part 1 study was conducted in healthy older adults as relevant age group for IPF. Overall, the head-to-head crossover study of Part 1 was designed at least in part to evaluate the tolerability impact of reducing exposure to the major metabolite. To this end, thirty-seven subjects were randomized in the blinded crossover study to receive 850 mg BID LYT-100 or 801 mg TID pirfenidone with three days of fed dosing and a 4^{th} day morning fasted dose. With reference to FIG. 5A, the Cₘₐₓ and AUC of parent drug for 850 mg BID LYT-100 were very similar to that of parent drug for 801 mg TID pirfenidone. Specifically, the steady-state AUC and with 850 mg BID dosing was 102% AUC compared with the steady-state AUC for pirfenidone dosed at 801 mg TID and the steady-state Cₘₐₓ achieved was 104% of the Cₘₐₓ of the steady-state Cₘₐₓ for pirfenidone dosed at 801 mg TID. Fasting increased the Cₘₐₓ. The major metabolite (5-carboxypirfenidone) exposure was reduced for 850 mg BID LYT-100 relative to that when pirfenidone was dosed at 801 mg TID.

The adverse events encountered in each treatment group are provided in FIG. 5B, which shows that no serious adverse events occurred in either group, and similar types of AEs were observed across both groups. No clinically meaningful differences between LYT-100 and pirfenidone in overall AE rates.

With reference to FIG. 5B, the adverse events in both groups were primarily GI and nervous system, with nervous system AEs including headache and dizziness. As noted above, fasting increased Cₘₐₓ and was hypothesized to increase overall GI AE rates. Consistent with this hypothesis, there was an increase in nausea in both groups when dosed after fasting, and the timing and duration of the AEs was consistent with a Cₘₐₓ-related effect. As illustrated in FIG. 5B, and with reference to FIG. 5A, the results of this study show that reducing exposure to the major metabolite did not improve tolerability.

### Part 2

Part 2 was a double-blind, randomized, two-period crossover study conducted in older healthy subjects to determine the safety, tolerability, and PK of 550 mg of LYT-100 administered three times daily (TID) for 3 days (to steady state [Day 1 to Day 3 and Day 11 to Day 13]) compared to pirfenidone administered 801 mg TID for 3 days (to steady state) under fed conditions. A final single dose of study drug (LYT-100 or pirfenidone) was administered on the morning of the fourth day in each treatment period (Day 4/Day 14) following an overnight fast of at least 8 hours to determine the effect of food on steady state PK parameters.

Overall, 49 subjects were enrolled and included in the Safety Population, 24 subjects to Sequence A and 25 subjects to Sequence B. Five subjects (10.2%) did not complete the study. Two subjects in Cohort 2 discontinued due to a TEAE (1 subject in Sequence A (LYT-100) and 1 subject in Sequence B (pirfenidone)). Two subjects in Cohort 2 discontinued due to physician decision (1 subject in Sequence A (LYT-100) and 1 subject in Sequence B (pirfenidone)). One subject in Cohort 1, randomized to Sequence A, withdrew consent while taking LYT-100.

The mean age of the overall population was 67.7; the mean age was similar in Cohorts 1 and 2 (68.5 and 66.9 years, respectively). The majority of subjects were female (53.1%; 52.2% in Cohort 1, 53.8% in Cohort 2), predominately white (81.6%), and the average BMI was 27.9 kg/m². The overall mean number of days of dosing with LYT-100 was 4.0 days (4.0 days in Cohort 1, 3.9 days in Cohort 2). The mean number of days of dosing with pirfenidone was 3.9 days (4.0 days in Cohort 1 and 3.9 days in Cohort 2).

Preliminary PK analyses have been conducted to assess the comparability of the exposure to parent (pirfenidone or deupirfenidone) and metabolite (5-carboxy pirfenidone, regardless of treatment) after administration of LYT-100 relative to after the administration of pirfenidone. Summary statistics of the key PK parameters, shown by analyte, fed status, and treatment, are shown in Table 10. Overall, exposure in terms of parent drug (AUC₀₋₂₄ and Cₘₐₓ) was slightly lower after administration of LYT-100 compared to pirfenidone and the time to Cₘₐₓ was slightly longer (median of 3 hours for LYT-100 and 2 hours for pirfenidone). Specifically, the Cₘₐₓ was about 20% lower for LYT-100 and did not meet criteria for bioequivalence. As expected, the major metabolite concentration was substantially lower after administration of LYT-100.

TABLE 10
Pharmacokinetic Parameters After Administration of Pirfenidone
or LYT-100 in Subjects Enrolled in Part 2

| | | | Cₘₐₓ | Tₘₐₓ | AUC₀₋₂₄ |
|---|---|---|---|---|---|
| Fed Status | Analyte | Treatment | (μg/mL) | (hr) | (μg*hr/mL) |
| Fed | Parent | Pirfenidone | 10.4 (37.5%) | 2.00 (0-12.0) | 150 (29.3%) |
| (Day 3/13) | | 801 mg TID | | | |
| | | LYT-100 550 | 8.33 (31.7%) | 3.00 (1.00-12.0) | 130 (30.7%) |
| | | mg TID | | | |
| | Metabolite | Pirfenidone | 6.82 (34.4%) | 2.00 (0-12.0) | 107 (28.1%) |
| | | 801 mg TID | | | |
| | | LYT-100 550 | 3.88 (26.4%) | 3.00 (0-12.0) | 65.0 (24.0%) |
| | | mg TID | | | |
| Fasted | Parent | Pirfenidone | 12.7 (23.5%) | 1.00 (0-2.00) | 44.1 (24.1%) |
| (Day 4/14) | | 801 mg TID | | | |
| | | LYT-100 550 | 9.17 (47.5%) | 1.00 (0-3.00) | 34.1 (49.7%) |
| | | mg TID | | | |
| | Metabolite | Pirfenidone | 7.52 (30.1%) | 1.00 (0-2.00) | 28.5 (29.7%) |
| | | 801 mg TID | | | |
| | | LYT-100 550 | 3.80 (45.7%) | 1.00 (0-2.00) | 15.5 (48.0%) |
| | | mg TID | | | |

Summary statistics shown as geometric mean (CV %) or median (min-max) for Tₘₐₓ
Note:
only subjects with sufficient concentration data for all periods/analytes included

The results of the bioequivalence assessment when the treatments were administered in the fed state (Days 3 or 13) are provided in Table 11. Despite the slightly lower exposure seen after administration of LYT-100 in the fed state, LYT-100 at a dose of 550 mg TID met the criteria for bioequivalence based on AUC₀₋₂₄ as the lower and upper limits of the 90% confidence interval for the geometric mean ratio fall within the required interval of 0.8 to 1.25.

TABLE 11
Bioequivalence Assessment using Data
from Subjects Enrolled in Part 2

| Parameter | Geometric Mean Ratio (Lower 5^{th}, Upper 95^{th}) |
|---|---|
| AUC₀₋₂₄ | 0.866 (0.831, 0.901) |
| Cₘₐₓ | 0.800 (0.737, 0.868) |

Note:
only subjects with sufficient concentration data for all periods/analytes included. Bioequivalence assessment was done using the method of Chow et al. as implemented in the BE package for R

Using the foregoing crossover data, a further simulation was performed. The simulation involved dose normalizing the observed AUC₀₋₂₄ after administration of LYT-100 in each subject to calculate the expected AUC₀₋₂₄ after administration of a hypothetical dose of 550 mg TID. The resultant AUC₀₋₂₄ was then compared to the observed AUC₀₋₂₄ after administration of pirfenidone 801 mg TID to calculate an individual ratio of LYT-100 to pirfenidone. These ratios were then assessed using the same process described in Chow (Design and Analysis of Bioavailability and Bioequivalence Studies; Chapman & Hall/CRC Biostatistics Series, Chapman; Hall/CRC 2008) and the CDER (Guidance for Industry Statistical Approaches to Establishing Bioequivalence Center for Drug Evaluation and Research [CDER], FDA, 2001). The results of the simulation are provided in Table 12. Based upon these assessments, an LYT-100 dose regimen of 550 mg TID is predicted to provide comparable parent drug exposure to pirfenidone dosed at 801 mg TID.

TABLE 12
Predicted Ratio of AUC0₋₂₄ and Cₘₐₓ (LYT-100:Pirfenidone
801 mg TID) after the Administration of Hypothetical
LYT-100 Dose using Pooled Data.

| | 90% Confidence Interval | |
|---|---|---|
| LYT-100 550 mg TID | AUC₀₋₂₄ | Cₘₐₓ |
| | 0.956 (0.926-0.986) | 0.764 (0.727-0.803) |

### Adverse Event Summary

Overall, 28 subjects (57.1%) experienced at least one TEAE; 14 (30.4%) while taking LYT-100 and 23 (48.9%) while taking pirfenidone. The most common TEAEs (>5% overall) were nausea, headache, dizziness, vomiting, and somnolence. A summary of these TEAEs, overall and by study medication, is provided in Table 13.

TABLE 13
Summary of the Most Common (>5%
Overall) TEAEs (Safety Population)

| | LYT-100 | Pirfenidone |
|---|---|---|
| | N = 46 | N = 47 |
| TEAE | n (%); # | n (%); # |
| Overall AEs | 14 (30.4); 29 | 23 (48.9); 50 |
| GI Disorders | 8 (17.4); 13 | 16 (34.0); 22 |
| Nausea | 7 (15.2); 8 | 14 (29.8); 16 |
| Vomiting | 2 (4.2); 4 | 3 (6.4); 3 |
| Nervous System | 8 (17.4); 9 | 15 (31.9); 22 |
| Disorders | | |
| Headache | 6 (13.0); 7 | 9 (19.1); 12 |
| Dizziness | 1 (2.2); 1 | 7 (14.9); 8 |
| Somnolence | 1 (2.2); 1 | 2 (4.3); 2 |

Overall TEAEs during LYT-100 dosing were mild for 10 subjects (21.7%; 19 events) and moderate for 4 subjects (8.7%; 10 events). Overall TEAEs during pirfenidone dosing were mild in 17 subjects (36.2%; 42 events) and moderate in 6 subjects (12.8%; 8 events). Overall, TEAEs leading to study discontinuation were reported by 2 (4.1%) subjects in Cohort 2, one while receiving LYT-100 (nausea) and one while receiving pirfenidone (headache and dizziness). No deaths or serious AEs were reported.

In this group of older adults (mean age=68) across the two treatment groups (LYT-100 at 550 mg TID vs pirfenidone at 801 mg TID, fed and fasted), the incidence of TEAE's was notably reduced in the LYT-100 treatment arm compared to the pirfenidone arm for nausea and dizziness. Overall, in subjects experiencing at least one TEAE, the incidence was substantially lower in the LYT-100 group than in the pirfenidone group. Specifically, there was a 38% reduction in the overall incidence of TEAEs with LYT-100 vs. pirfenidone (30.4% versus 48.9%, respectively).

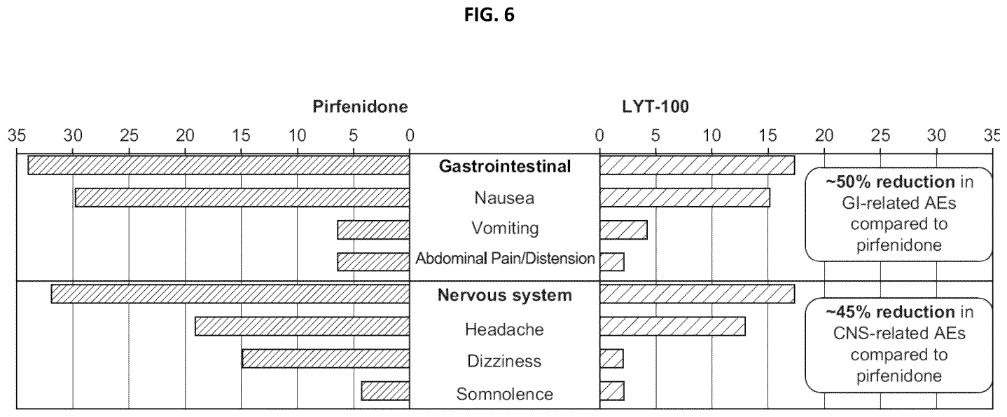

FIG. 6 provides a graphical illustration of the reduction in GI and nervous system symptoms for LYT-100 at 550 mg TID versus pirfenidone at 801 mg TID in this patient population. With reference to FIG. 6, fifty percent fewer subjects experienced GI-related AEs with LYT-100 compared to pirfenidone (17.4% versus 34.0%, respectively), including 50% fewer with nausea (15.2% versus 29.8%). Fewer subjects experienced nervous system AEs with LYT-100 compared to pirfenidone (17.4% vs. 31.9%), notably dizziness (2.2% with LYT-100 versus 14.9% versus pirfenidone). These study results show that substantially fewer subjects taking LYT-100 experienced AEs compared with pirfenidone and approximately 50% fewer subjects experienced GI-related AEs with LYT-100 compared with pirfenidone. There were no differences in the incidence of study discontinuation between the treatment groups. The results suggest that LYT-100 may be better tolerated at 550 mg TID than pirfenidone 801 mg TID in this subject population.

With respect to fed versus fasted condition prevalence of TEAEs, there were 8 (17.4%) LYT-100-treated subjects who experienced at least 1 TEAE under fed conditions and 8 (17.8%) subjects under fasted conditions. There were 10 (21.3%) pirfenidone-treated subjects under fed conditions and 17 (37.0%) subjects under fasted conditions who experienced at least one TEAE. A summary of the most common TEAEs (≥10%) under fed and fasted conditions is provided in Table 14 for each study medication.

TABLE 14
Summary of the Most Common TEAEs (≥5%) under
Fed and Fasted Conditions (Safety Population)

| | LYT-100 550 mg TID n (%) | | Pirfenidone 801 mg TID n (%) | |
|---|---|---|---|---|
| | Fed | Fasted | Fed | Fasted |
| TEAE | (N = 46) | (N = 45) | (N = 47) | (N = 46) |
| GI Disorders | 3 (6.5) | 5 (11.1) | 5 (10.6) | 13 (28.3) |
| Nausea | 2 (4.3) | 5 (11.1) | 4 (8.5) | 12 (26.1) |
| Vomiting | 2 (4.3) | 5 (11.1) | 2 (4.3) | 1 (2.2) |
| Nervous System | 4 (8.7) | 4 (8.9) | 7 (14.9) | 10 (21.7) |
| Disorders | | | | |
| Headache | 4 (8.7) | 2 (4.4) | 5 (10.6) | 5 (10.9) |
| Dizziness | 0 | 1 (2.2) | 3 (6.4) | 5 (10.9) |

In this study, fed conditions reduced the incidence of TEAEs in both treatment arms. In addition, LYT-100 was better tolerated in both the fed and fasted conditions than pirfenidone within these two dose groups. This improved tolerability of LYT-100 also seems to be amplified in the fasted state. Without wishing to be bound by any particular theory, it is believed that the greater incidence of TEAEs experienced by fasting subjects in both treatment groups may be causally related to the higher peak plasma concentrations (Cₘₐₓ) of the parent molecules (pirfenidone or deupirfenidone) that result from their more rapid and extensive absorption during fasting than when taken with food. A causal role for higher Cₘₐₓ in tolerability is suggested by the observations that on the days of fasting, Cₘₐₓ was higher in both treatment groups, and the incidence of TEAEs was substantially greater for both treatment groups than on fed days. In addition, it is notable that the fasting increase in Cₘₐₓ in the pirfenidone group was associated with the greatest incidence of TEAEs in the study. Overall, the head-to-head crossover study of Part 2 was designed at least in part to evaluate the tolerability impact of reducing the parent Cₘₐₓ. As described herein above, the results of this study show that reducing the parent drug Cₘₐₓ improves tolerability.

### Example 2: LYT-100 Crossover Study-550 and 824 mg TID

This study was a double-blind, randomized, two-period crossover study in older, healthy subjects to compare the safety, tolerability, and pharmacokinetics of deupirfenidone (LYT-100) and pirfenidone. The crossover study was performed at a single Study Center per Part in the United States.

### Study Description

This study was a randomized, double-blinded, parallel arm, placebo-controlled study conducted in healthy older adults to evaluate the safety and tolerability compared to placebo of a dose of LYT-100 that provides an exposure of LYT-100 which is approximately 150% of the exposure of pirfenidone when dosed at 801 mg TID and did not exceed 850 mg TID LYT-100.

### Study Endpoints

### Safety:

Treatment-emergent adverse events (TEAEs), including severity, and relatedness to study drug)
Physical examination
Vital signs
Electrocardiograms (ECGs)
Clinical laboratory parameters, including hematology, serum chemistry, coagulation, and urinalysis
New-onset concomitant medications

### Pharmacokinetics:

Comparison of the key PK parameters (C_{max,ss}, C_{min,ss}, and AUC_{0-24,ss}) between the parent compound (LYT-100 and pirfenidone) and primary metabolite (5-carboxypirfenidone). Other PK parameters will also be derived and compared.
Comparison of the key urine PK parameters (Aeₜ₁₋ₜ₂, CL_{R}, Feₜ₁₋ₜ₂) between the parent compound (LYT-100 and pirfenidone) and primary metabolite (5-carboxypirfenidone). Other urine PK parameters may be derived and compared.
Food effect evaluation of LYT-100 and pirfenidone (C_{max,ss}, and AUC_{0-6,ss}) for fed versus fasted.

### Study Design

This was a randomized, double-blinded, parallel arm, placebo-controlled study conducted in healthy older adults to evaluate the safety and tolerability of titrated high dose LYT-100 compared to placebo under fed conditions. Thirty older healthy adults between the ages of 60 and 80 were randomized to receive LYT-100 or placebo. Subjects were administered 550 mg LYT-100 three times daily (TID) for 3 days (to steady state [Day 1 to Day 3]) compared to 550 mg placebo administered TID for 3 days to steady state. Day 4 to Day 6, subjects were administered 824 mg LYT-100 TID for 3 days compared to 824 mg placebo TID for 3 days to steady state. Informed consent was obtained prior to the commencement of the study. Screening was performed up to 28 days prior to administration of the first dose of LYT-100/placebo. Only subjects who met all the applicable inclusion and none of the applicable exclusion criteria were randomized. The dosing schedule is outlined in Table 15.

TABLE 15
Dosing Regimen and Treatment Sequence

| | Dose, Days | Daily | Dose, Days | Daily |
|---|---|---|---|---|
| N | 1 to 3 | total dose | 4 to 6 | total dose |
| 24 | LYT-100, | LYT-100, | LYT-100, | LYT-100, |
| | 550 mg TID | 1650 mg | 824 mg TID | 2427 mg |
| 6 | Placebo, | Placebo, | Placebo, | Placebo, |
| | 550 mg TID | 1650 mg | 824 mg TID | 2427 mg |

### Number of Subjects:

Thirty healthy older female and male adult subjects (target ratio 1:1 of males: females with a minimum of 10 per sex per cohort)

### Main Criteria for Inclusion and Exclusion

### Inclusion Criteria:

1. Male or female between 60 and 80 years old (inclusive) at the time of screening.
2. Subjects have a body mass index (BMI) between ≥18.0 and ≤35.0 kg/m² at screening.
3. Willing and able to abstain from direct whole body sun exposure from 2 days prior to dosing and until final study procedures have been conducted. Subjects should be instructed to avoid or minimize exposure to sunlight (including sunlamps), use an SPF 50 sun block, or higher, wear clothing that protects against sun exposure and avoid concomitant medications known to cause photosensitivity (including but not limited to tetracycline, doxycycline, nalidixic acid, voriconazole, amiodarone, hydrochlorothiazide, naproxen, piroxicam, chlorpromazine and thioridazine).

### Exclusion Criteria:

1. Pregnant or lactating at screening or baseline or planning to become pregnant (self or partner) at any time during the study, including the specified follow-up period.
2. History or presence of malignancy at screening or baseline, with the exception of adequately treated localised skin cancer (basal cell or squamous cell carcinoma) or carcinoma in-situ of the cervix.
3. Clinically significant infection within 28 days of the start of dosing, or infections requiring parenteral antibiotics within the 3 months prior to screening. Known exposure to another person with COVID-19 within the last 14 days is also an exclusion criterion, or a positive COVID test within five days prior to dosing.
4. Had major surgery, (e.g., requiring general anesthesia) within 3 months before Screening, based on Investigator's discretion or has surgery planned during the time the participant is expected to participate in the study.
5. Currently suffering from clinically significant systemic allergic disease at screening or baseline or has a history of significant drug allergies including a history of anaphylactic reaction (particularly reactions to general anaesthetic agents); allergic reaction due to any drug which led to significant morbidity; prior allergic reaction to pirfenidone.
6. Chronic administration (defined as more than 14 consecutive days) of immunosuppressants or other immune-modifying drugs within 3 months prior to study drug administration. Corticosteroids are permitted at the discretion of the Investigator.
7. History or presence at screening or baseline of a condition associated with significant immunosuppression.
8. Positive test for hepatitis C antibody (HCV), hepatitis B surface antigen (HBsAg), or human immunodeficiency virus (HIV) antibody at screening.
9. Symptoms of dysphagia at screening or baseline or known difficulty in swallowing capsules.
10. Any condition at screening or baseline (e.g., chronic diarrhoea, inflammatory bowel disease or prior surgery of the gastrointestinal tract) that would interfere with drug absorption or any disease or condition that is likely to affect drug metabolism or excretion, at the discretion of the Investigator.
11. History or presence at screening or baseline of cardiac arrhythmia or congenital long QT syndrome.
12. QT interval corrected using Fridericia's formula (QTcF) >450 msec. ECG may be repeated 30 to 60 minutes apart from the first one collected at screening. If repeat ECG is ≤450 msec, the second ECG may be used to determine patient eligibility. However, if repeat ECG confirms QTcF remains >450 msec, the subject is not eligible.
13. Use of tobacco or nicotine containing products in the previous 3 months prior to dosing or a positive urine cotinine test at Screening or Baseline.
14. Lack of willingness to abstain from the consumption of tobacco or nicotine-containing products throughout the duration of the study and until completion of the final Follow-up visit.
15. Regular alcohol consumption defined as >21 alcohol units per week (where 1 unit=284 mL of beer, 25 mL of 40% spirit or a 125 mL glass of wine) or the Participant is unwilling to abstain from alcohol for 48 h prior to admission and 48 h prior to study visits.
16. Use of any of the following drugs within 30 days or 5 half-lives of that drug, whichever is longer, prior to study drug administration:
   a. Fluvoxamine, enoxacin, ciprofloxacin;
   b. Other inhibitors of CYP1A2 (including but not limited to methoxsalen or mexiletine);
   c. Contraceptives containing oestradiol, ethinyloestradiol or gestodene;
   d. Inducers of CYP1A2 (such as phenytoin), CYP2C9 or 2C19 (including but not limited to carbamazepine or rifampin);
   e. Any drug associated with prolongation of the QTc interval (including but not limited to moxifloxacin, quinidine, procainamide, amiodarone, sotalol).
17. Vaccination with a live vaccine within the 4 weeks prior to screening or that is planned within 4 weeks of dosing, and any non-live vaccination within the 2 weeks prior to screening or that is planned within 2 weeks of dosing (including those for COVID).
18. Use of any investigational drug or device within the longer of 30 days or five half-lives prior to screening.
19. Consumption of grapefruit, grapefruit juice, Seville oranges, Seville orange juice, or any foods containing these ingredients, within 7 days prior to dosing or unwilling to abstain from these throughout the duration of the study.

### Dosage and Mode of Administration:

This was a crossover study in which subjects received both the test treatment (LYT-100) and the reference (pirfenidone).
LYT-100 (Deupirfenidone) was provided as hard gelatin capsules. LYT-100 should be stored at a controlled room temperature of 15° C. to 25° C.
Pirfenidone (Esbriet) was provided as white to off-white hard gelatin capsules contain 267 mg of pirfenidone. The cap of the capsule is printed with “PFD 267 mg” in brown ink. Pirfenidone should be stored at 15° C. to 25° C.
Both LYT-100 and pirfenidone were over-encapsulated to maintain study blind.

### Duration of Treatment:

This study included a 28-day Screening period, a 6-day treatment period consisting of: 3 days of up to 550 mg TID LYT-100 followed directly by 3 days of 824 mg TID LYT-100, or placebo. A 3-day (±1 day) post-last-dose safety follow-up visit occurred. Thus, total duration of study participation for each subject was up to 40 days.

### Criteria for Evaluation

### Safety:

Safety and tolerability were assessed by monitoring AEs, physical examination, vital signs, 12-lead ECGs, clinical laboratory values (hematology panel, multiphasic chemistry panel and urinalysis), and review of concomitant treatments/medication use.

### Pharmacokinetics:

Subjects provided blood samples prior to treatment, i.e., Day −1 or Day 1, for the determination of CYP1A2, CYP2C9, CYP2C19, and CYP2D6 genotype to support exploratory PK analyses. Subjects were required to provide consent for genotyping. Blood samples for PK were collected at specified times, as follows:
Day 1: 0 (pre-AM dose)
Day 2: no sampling
Day 3: 0 (pre-AM dose), and 1, 2, 3, 4, 6 (pre-mid-day dose), 7, 8, 9, 10, 12 (pre-PM dose), 13, 14, 15, 16, and 17 hours post-AM dose
Day 4: 0 (pre-AM dose)
Day 5: no sampling
Day 6: 0 (pre-AM dose), and 1, 2, 3, 4, 6 (pre-mid-day dose), 7, 8, 9, 10, 12 (pre-PM dose), 13, 14, 15, 16, and 17 hours post-AM dose
Day 7: 0 (same time as Day 6 pre-AM dose, discharge)

Plasma concentration-time data for LYT-100, and its metabolite(s) were analyzed using noncompartmental methods. Plasma PK parameters for steady state dosing (Days 1 to 3 and Days 4 to 7) included, but were not limited to:
AUC_{0-tau,ss} (area under the time concentration curve from time zero to tau at steady state)
AUC_{0-24,ss} (area under the time concentration curve from time zero to 24 hours at steady state)
λ_{z} (terminal disposition rate constant/terminal rate constant)
t_{1/2} (elimination half-life)
C_{max,ss} (maximum concentration in a dosing interval)
Tₘₐₓ (time to maximum concentration, as reported relative to the beginning of a dosing interval in which maximum concentration occurred)
C_{min,ss} (lowest concentration in a dosing interval)
C_{av,ss} (average concentration during a dosing interval)
C_{max,ss}−C_{min,ss}/C_{av,s}s (degree of fluctuation)
C_{max,ss}−C_{min,ss}/C_{min,ss} (swing)
PTF % (peak-trough fluctuation)

Urine samples for PK were collected at specified intervals, as follows:
Days 1 and 4: pre-dose (subjects to be instructed to empty their bladders approximately 30 minutes prior to dosing)
Days 2 and 5: no urine sampling
Days 3 and 6: pre-dose (subjects to be instructed to empty their bladders approximately 30 minutes prior to dosing), 0 to 4, 4 to 8, 8 to 12, 12 to 16, and 16 to 24 hours post-AM dose

Urine samples for analysis of excretion in urine will be collected, separated by specified time interval, and analyzed. The total volume of urine collected in each interval (t1 to t2) will be noted. The urine PK parameters included, but were not limited to:
Aeₜ₁₋ₜ₂ (Amount excreted in urine over time)
CLR (Renal clearance)
Fraction of systemic clearance (CL/F) represented by the renal clearance (CLR/[CL/F])
Feₜ₁₋ₜ₂ (Fraction of administered dose excreted in urine over the dosing intervals)
   Study endpoints are defined as follows:

### Safety

AEs (type, severity, and relatedness to study drug)
Physical examination
Vital signs
Electrocardiograms (ECGs)
Clinical laboratory parameters (hematology, serum chemistry, coagulation, and urinalysis)
New-onset concomitant medications

### Pharmacokinetics:

Comparison of the key plasma PK parameters (C_{max,ss}, C_{min,ss}, and AUC_{0-24,ss}) between the parent compound (LYT-100 and pirfenidone) and primary metabolite (5-carboxypirfenidone). Other plasma PK parameters were also derived and compared.
Comparison of the key urine PK parameters (Aeₜ₁₋ₜ₂, CL_{R}, Feₜ₁₋ₜ₂) between the parent compound (LYT-100 and pirfenidone) and primary metabolite (5-carboxypirfenidone). Other urine PK parameters may have bene derived and compared.
Food effect evaluation of LYT-100 and pirfenidone (C_{max,ss}, and AUC_{0-6,ss}) for fed vs fasted.

### Results—Pharmacokinetics

Based on the observations of improved tolerability (but comparable total exposure) for a lower TID dose of LYT-100 compared to pirfenidone in Example 1, the safety and tolerability of a higher TID dose of LYT-100 (to achieve a higher overall predicted AUC or total exposure than the approved dose of pirfenidone (801 mg TID), and to explore the possibility of evaluating that dose in future efficacy studies), was evaluated in this study.

Subjects between the ages of 60 and 80 were randomized to receive LYT-100 or placebo. Subjects were administered up to 550 mg LYT-100 TID for 3 days (to steady state [Day 1 to Day 3]) compared to placebo administered TID for 3 days to steady state. On Day 4 to Day 6, subjects were administered 824 mg LYT-100 TID for 3 days compared to placebo TID for 3 days to steady state. A summary of the dosing scheme is provided in Table 16.

TABLE 16
Dosing Scheme

| Number of | Dose, | Total | Dose, | Total |
|---|---|---|---|---|
| Subjects | Days 1 to 3 | Daily Dose | Days 4 to 6 | Daily Dose |
| 24 | LYT-100, | 1650 mg | LYT-100, | 2472 mg |
| | 550 mg TID | | 824 mg TID | |
| 6 | Placebo TID | — | Placebo TID | — |

Overall, 30 subjects were enrolled and included in the Safety Population, 24 subjects to LYT-100 and 6 subjects to placebo. Seven subjects (23.3%) did not complete the study. The mean age of the overall population was 64.9; the mean age was similar in the LYT-100 and placebo groups, 65.0 and 64.5 years, respectively. The majority of subjects were male (56.7%; 66.7% in the LYT-100 group, 16.7% in the placebo group). The overall mean number of days of dosing with LYT-100 was 5.5 days. The mean number of days of dosing with placebo was 5.8 days.

Data was obtained for thirty subjects. Eight subjects had all values reported as below level of quantitation (BLQ; assumed to be 6 placebo subjects plus 2 active subjects with Day 1 pre-dose samples only). One additional subject was excluded due to a large number of BLQ samples on both Days 3-4 and 6-7. Accordingly, twenty-one subjects had sufficient PK data available to calculate PK parameters at the lower dose (550 mg TID on Days 3-4). Three subjects only had data for Days 3-4, and therefore had missing PK parameters for the higher dose (824 mg TID on Days 6-7).

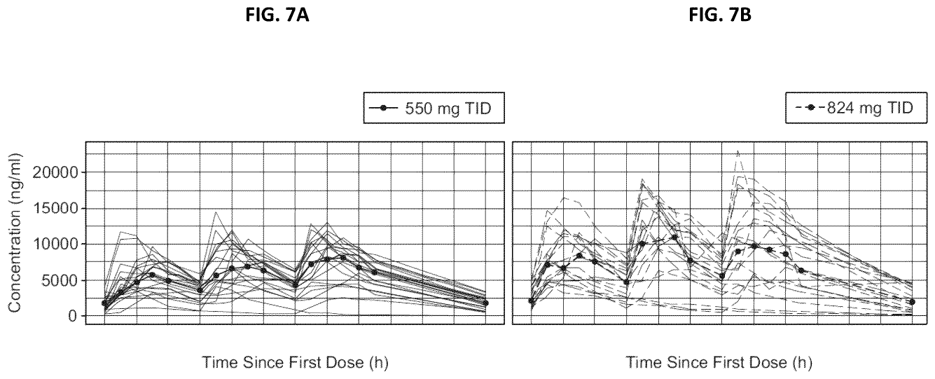

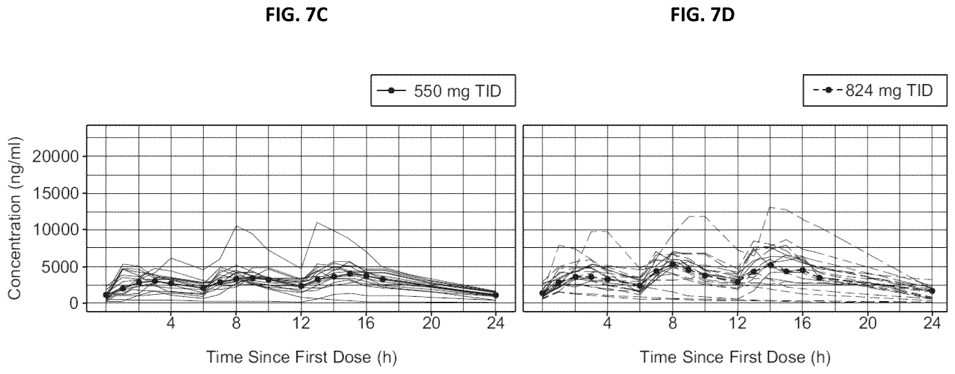

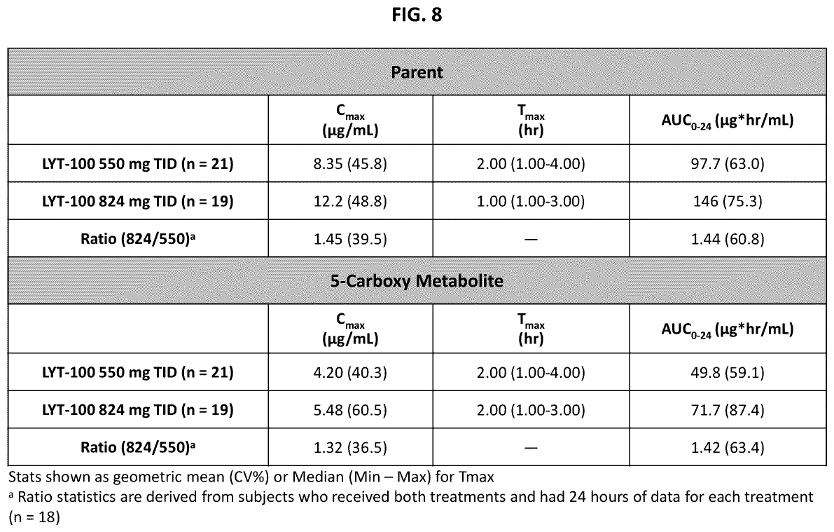

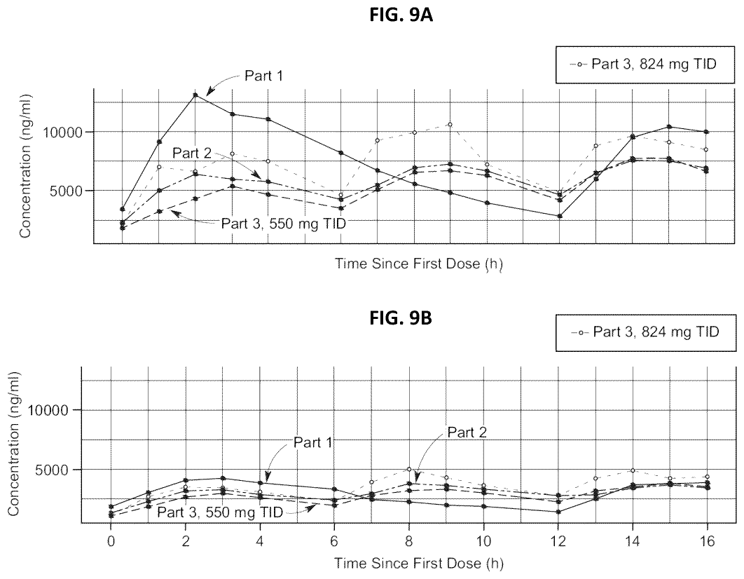

The results for the pharmacokinetic assessments are provide in FIG. 7A to FIG. 18B. With reference to FIGS. 7A-7D, the plasma concentrations for both the parent drug (LYT-100; SD-560) and major metabolite (5-carboxypirfenidone; SD-789) were higher for the 824 mg TID dose cohort (FIGS. 7B and 7D) relative to the 550 mg TID dose cohort (FIGS. 7A and 7C). The Cₘₐₓ, AUC, and Tₘₐₓ values in the fed state for LYT-100 and the major metabolite at the 550 mg and 824 mg TID doses are provided in FIG. 89. With reference to FIG. 8, for LYT-100, the Cₘₐₓ ratio for the 824 mg TID to the 550 mg TID dose was 1.45, and the AUC ratio was 1.44, demonstrating an approximately linear dose-exposure relationship. The Cₘₐₓ and AUC ratios for the metabolite were slightly reduced at 1.32 and 1.42, respectively.

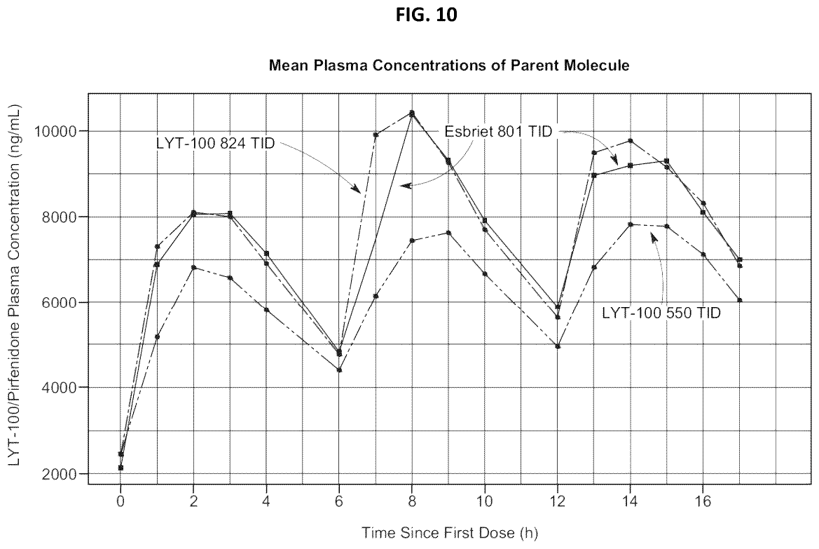

The results for this study were compared to the results obtained in a prior 850 mg BID study and a prior 550 mg TID study (described herein in Example 1). As shown in FIGS. 10A and 10B (LYT-100 and major metabolite, respectively), although slightly lower, the AUC for the present 550 mg TID (days 1-3) study roughly matches up with the AUC of 550 mg TID from the prior 550 mg TID study (part 2; solid blue and solid green lines respectively; see also Example 1, Table 13), and the AUC and Cₘₐₓ for the 824 mg TID dose shows a pronounced/linear increase over that for the 550 mg TID dose.

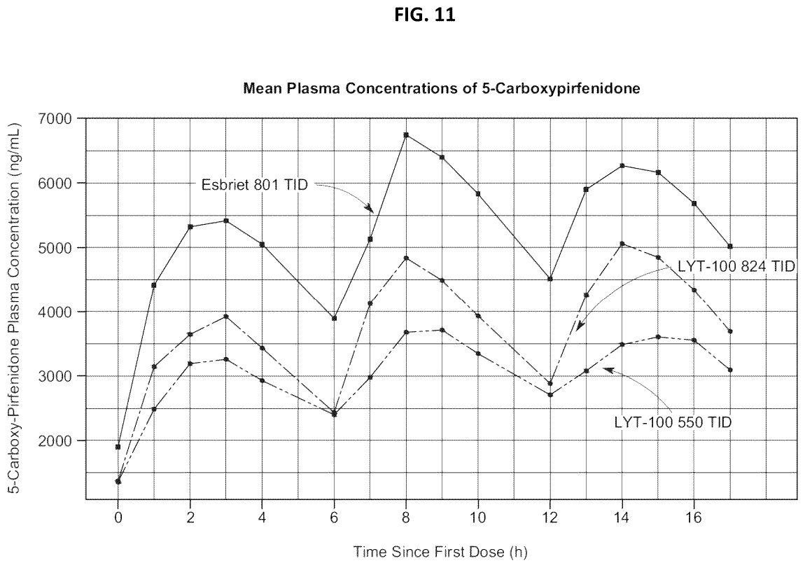

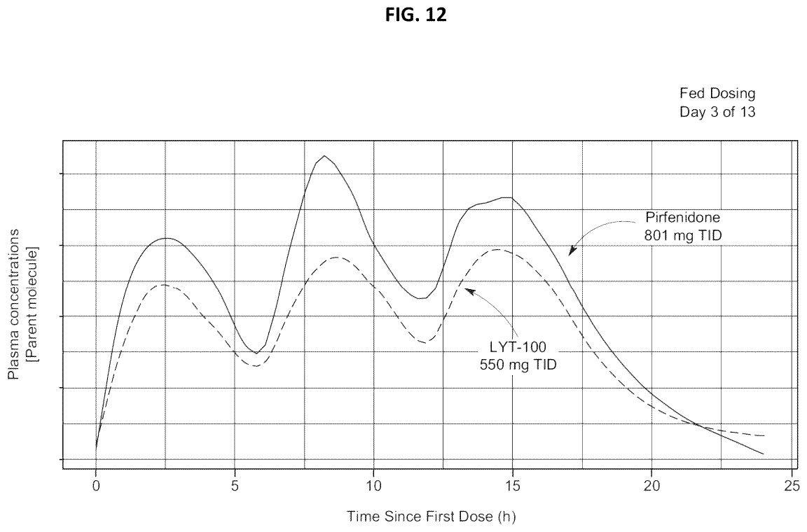

FIG. 10 provides a comparison of plasma concentrations of LYT-100 (dosed at 550 mg and 824 mg TID) and pirfenidone (dosed at 801 mg TID) versus time following the day 3 doses. With reference to FIG. 10, the concentration peaks for pirfenidone are higher than those for 550 mg LYT-100. FIG. 11 provides a comparison of plasma concentrations of the major metabolite of LYT-100 (dosed at 550 mg and 824 mg TID) and pirfenidone (dosed at 801 mg TID) versus time following the day 3 doses. FIG. 12 provides a comparison of plasma concentrations versus time for pirfenidone at 801 mg TID and LYT-100 at 550 mg TID following the day 3 doses.

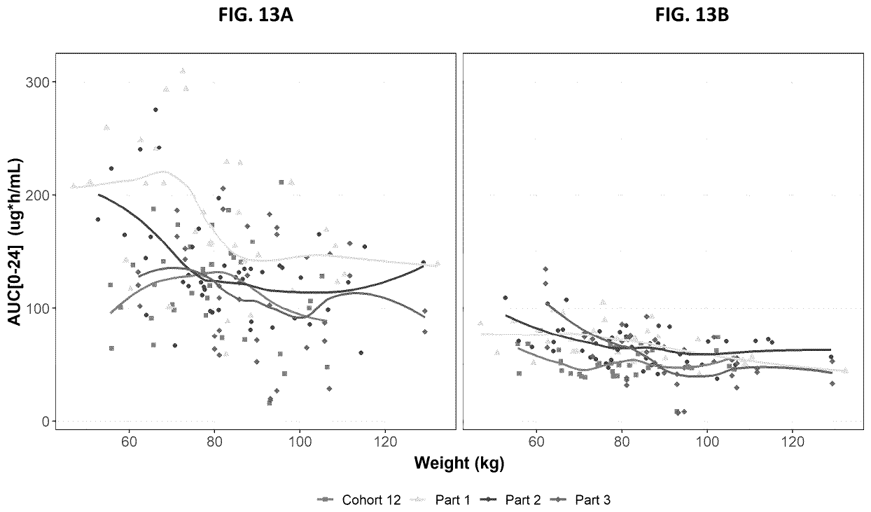

FIG. 13A provides a comparison of AUC₀₋₂₄ versus body weight for LYT-100 administration across this and previous studies. FIG. 13B provides a comparison of AUC₀₋₂₄ versus body weight for the major metabolite of LYT-100 across this and previous studies. With reference to FIGS. 13A and 13B, a similar trend for impact of body weight was observed across all three groups, with an apparent exposure difference above and below a threshold of 70-75 kg.

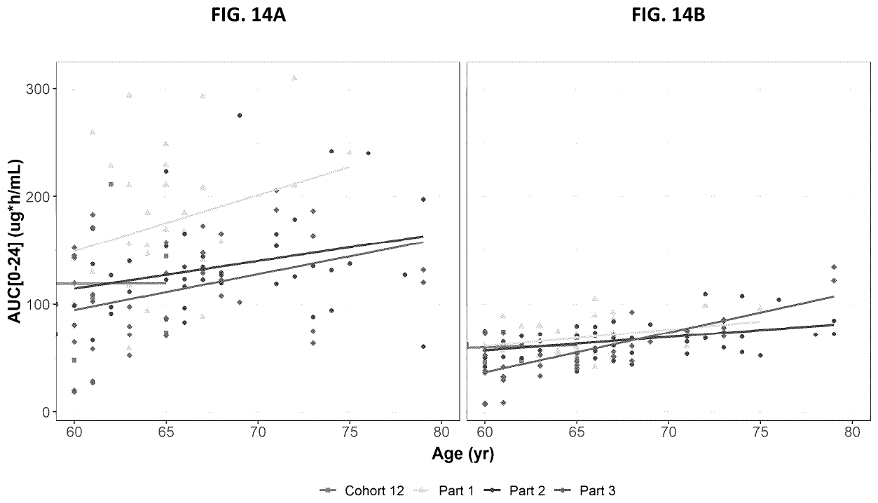

FIGS. 14A and 14B provide a comparison of AUC₀₋₂₄ versus subject age for LYT-100 and the major metabolite, respectively, across this and previous studies. With reference to FIGS. 14A and 14B, age appears to impact AUC, with exposure increasing with age.

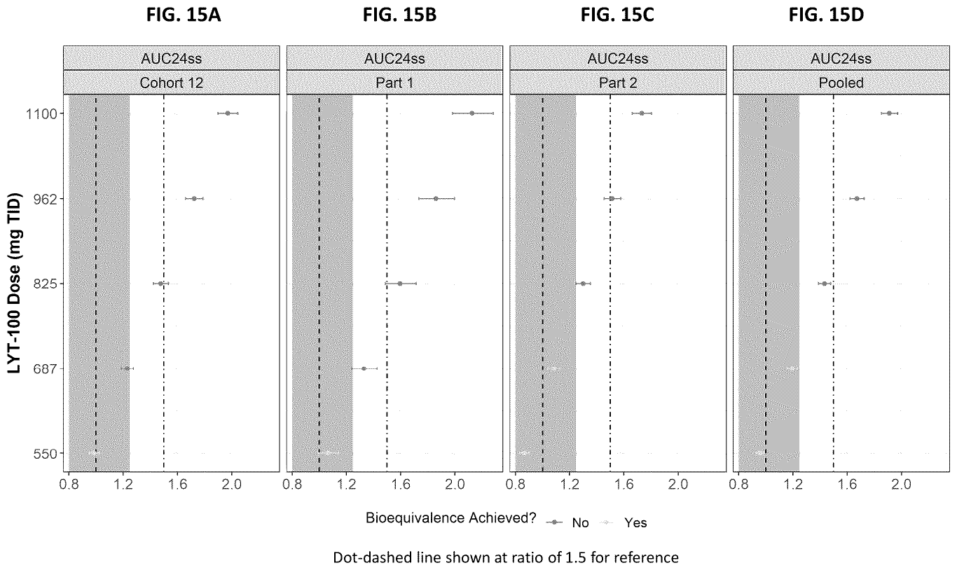

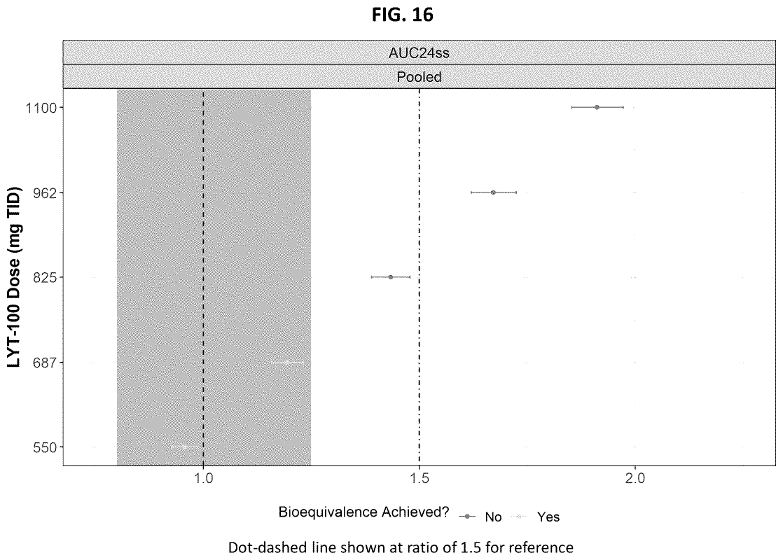

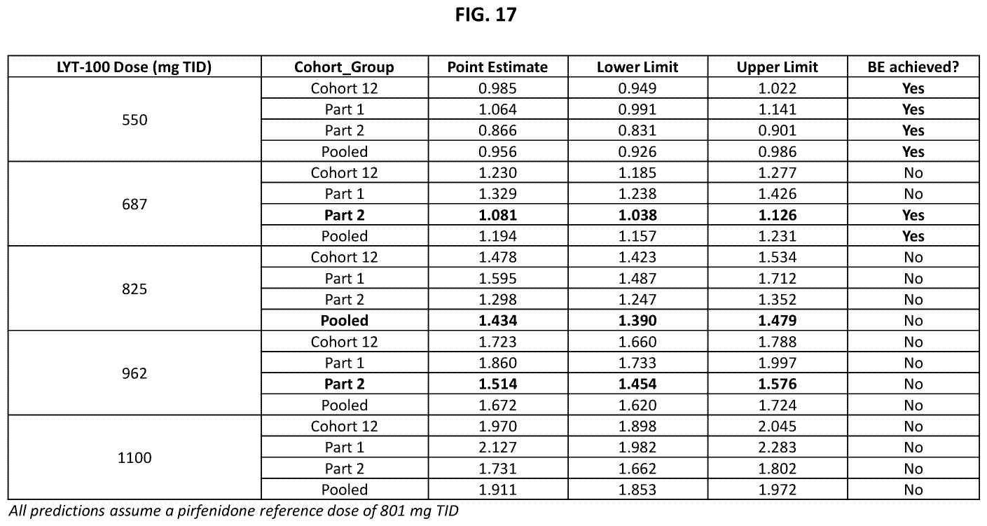

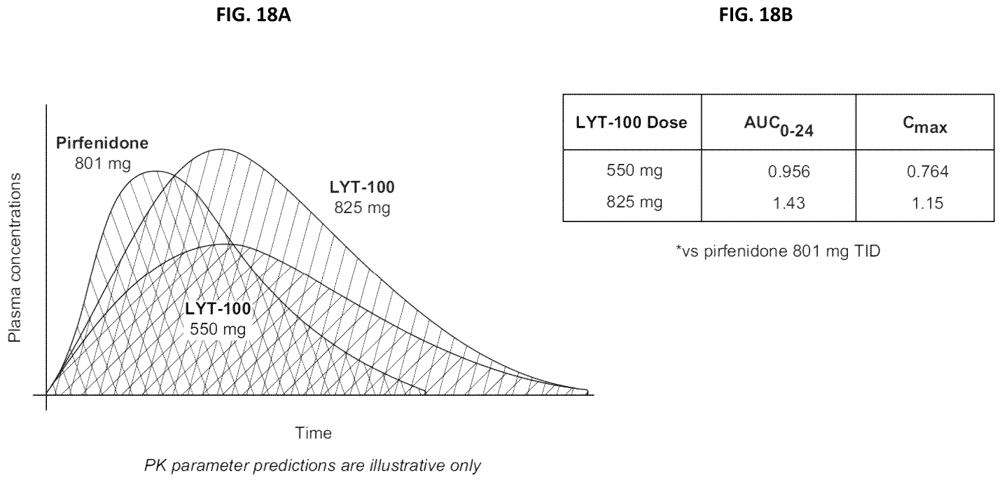

Bioequivalence simulations were performed for AUC₂₄ₛₛ across this dosing study and three prior dosing studies. Results are provided in FIGS. 15A-15D and FIG. 16, which show that bioequivalence to 801 mg TID pirfenidone was achieved for 550 mg TID LYT-100 when pooled data from the studies was used, and bioequivalence was observed for a theoretical 687 mg TID dose (FIG. 16). The results of the simulations across this study and three prior studies is provided in tabular form in FIG. 17. An illustrative prediction of plasma concentration over time for theoretical 550 mg TID and 825 mg TID dosing of LYT-100 and 801 mg TID dosing of pirfenidone is provided in FIG. 18A. With reference to FIG. 18A, it is predicted that for the 550 mg TID dosing, the maximal plasma concentration (Cmax) of LYT-100 achieved is less than the maximal plasma concentration of pirfenidone achieved with 801 mg TID dosing, but with a similar exposure (AUC). In contrast, it is predicted that with the 825 mg TID dosing, the Cmax of LYT-100 achieved is only slightly more than the maximal plasma concentration of pirfenidone achieved with 801 mg TID dosing, but with a higher AUC. The estimated Cmax and AUC ratios of LYT-100 to pirfenidone are provided in FIG. 18B.

### Results—Tolerability

Four subjects discontinued due to an TEAE (3 (12.5%) subjects in the LYT-100 group and 1 (16.7%) subject in the placebo group). Three (12.5%) subjects withdrew consent; all were in the LYT-100 group. Overall, 9 subjects (30.0%) experienced at least one TEAE; 8 (33.3%) while taking LYT-100 and 1 (16.7%) while taking placebo. The most common TEAEs (>5% overall) were COVID-19 and headache. A summary of these TEAEs, overall and by study medication, is provided in Table 17.

TABLE 17
Summary of the Most Common (>5%
Overall) TEAEs (Safety Population)

| | LYT-100 | Placebo | Overall |
|---|---|---|---|
| | N = 24 | N = 6 | N = 30 |
| TEAE | n (%) | n (%) | n (%) |
| COVID-19 | 4 (16.7) | 1 (16.7) | 5 (16.7) |
| Headache | 3 (12.5) | 0 | 3 (10.0) |

A summary of TEAEs stratified by onset day 1 to 3 or day 4 to 6 showed that the onset of the COVID-19 events occurred within days 4 to 6; the onset of the headache events occurred within days 1 to 3. Overall, the majority of TEAEs were considered to be mild. There were 13 mild events reported by 8 (26.7%) subjects, 7 (29.2%) in the LYT-100 group and 1 (16.7%) in the placebo group. Two moderate TEAEs were reported by one (3.3%) subject; this subject was in the LYT-100 group. No TEAEs were severe. TEAEs were unrelated for 5 (16.7%) events and possibly related for 6 (13.3%) events. No events were probably related. Overall, TEAEs leading to study discontinuation were reported by 4 (13.3%) subjects; all were Covid-19. Of these 4 TEAEs, 3 (12.5%) occurred in the LYT-100 group and 1 (16.7%) occurred in the placebo group. No deaths or SAEs were reported during the study.

Based on prior PK modeling studies, the AUC of LYT-100 at 824 mg TID is expected to be approximately 150% of the AUC for the approved pirfenidone dose of 801 mg TID. Within the 824 mg TID LYT-100 group (mean age=65), the dose was well tolerated over the 3 treatment days. In this dosage group, the most common TEAE was headache, and the majority of the events were mild.

Using the foregoing crossover data, a further simulation was performed. The simulation involved dose normalizing the observed AUC₀₋₂₄ after administration of LYT-100 in each subject to calculate the expected AUC₀₋₂₄ after administration of various hypothetical TID doses. The resultant AUC₀₋₂₄ was then compared to the observed AUC₀₋₂₄ after administration of pirfenidone 801 mg TID to calculate an individual ratio of LYT-100 to pirfenidone. These ratios were then assessed using the same process described in Chow (Design and Analysis of Bioavailability and Bioequivalence Studies; Chapman & Hall/CRC Biostatistics Series, Chapman; Hall/CRC 2008) and the CDER (Guidance for Industry Statistical Approaches to Establishing Bioequivalence Center for Drug Evaluation and Research [CDER], FDA, 2001). The results of the simulation are provided in Table 18. Based upon these assessments, an LYT-100 dose regimen of 550 mg TID is predicted to provide comparable parent drug exposure to pirfenidone dosed at 801 mg TID. An LYT-100 dose regimen of 825 mg TID is predicted to provide parent drug exposure that is approximately 150% of that following administration of pirfenidone given 801 mg TID. Of note, the slower absorption of LYT-100 relative to pirfenidone results in a predicted Cₘₐₓ for LYT-100 at a dose of 825 mg TID that is only 15% higher than the corresponding Cₘₐₓ for pirfenidone at a dose of 801 mg TID.

The actual and extrapolated exposure and Cₘₐₓ values for LYT-100 dosed at 550 and 824/825 mg TID, along with the tolerability data, support these two doses for studying the efficacy, safety, and dose response in idiopathic pulmonary fibrosis, as described below in Example 3.

TABLE 18
Predicted ratio of AUC₀₋₂₄ and Cₘₐₓ × (LYT-100:pirfenidone
801 mg TID) after the administration of various actual
and hypothetical LYT-100 doses using pooled data

| | |
|---|---|
| LYT-100 Dose | 90% Confidence Interval |

| (mg TID) | AUC₀₋₂₄ | Cₘₐₓ |
|---|---|---|
| 550 | 0.956 (0.926-0.986) | 0.764 (0.727-0.803) |
| 687 | 1.19 (1.16-1.23) | 0.955 (0.908-1.00) |
| 825 | 1.43 (1.39-1.48) | 1.15 (1.09-1.21) |
| 962 | 1.67 (1.62-1.72) | 1.34 (1.27-1.41) |
| 1100 | 1.91 (1.85-1.97) | 1.53 (1.46-1.61) |

### Example 3: LYT-100 Tolerability in Patients with COVID-19 Respiratory Illness

A Phase 2 multi-center randomized, double-blind, parallel arm, placebo-controlled trial was performed to evaluate the safety and efficacy of deupirfenidone (LYT-100) compared to placebo in post-acute adult patients with COVID-19 respiratory disease who were treated with supplemental oxygen (including MV, ECMO or any other means of oxygen administration) in the hospital for at least 1 day and have required only low flow nasal oxygen or no oxygen supplementation for at least 72 hours prior to screening. Patients received LYT-100 (deupirfenidone) formulated as powder in 250 mg capsules or matching placebo. Dosing was as provided in Table 19. An initial dosage of 500 mg BID was given the first 3 days of dosing, followed by 750 mg BID thereafter. Patients took LYT-100 study medication, or placebo (in Part A), orally and preferably with food, (solid or nutritional supplements, whenever possible), with approximately 10 to 12 hours between the two daily doses.

TABLE 19
Dosing Regimens

| Day 1 to Day 3 | Day 4 through Day 91 |
|---|---|
| LYT-100 500 mg BID or | LYT-100 750 mg BID or |
| matching Placebo × 3 days | matching Placebo × 88 days |

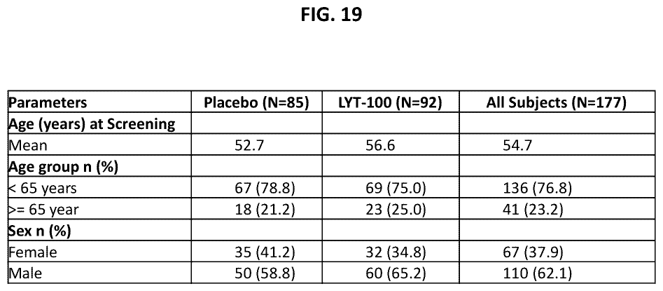

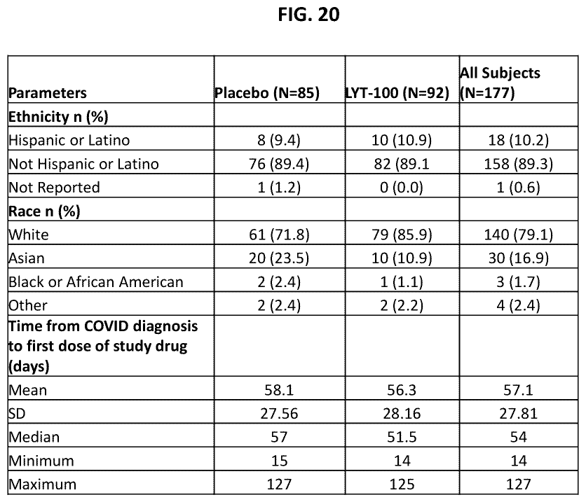

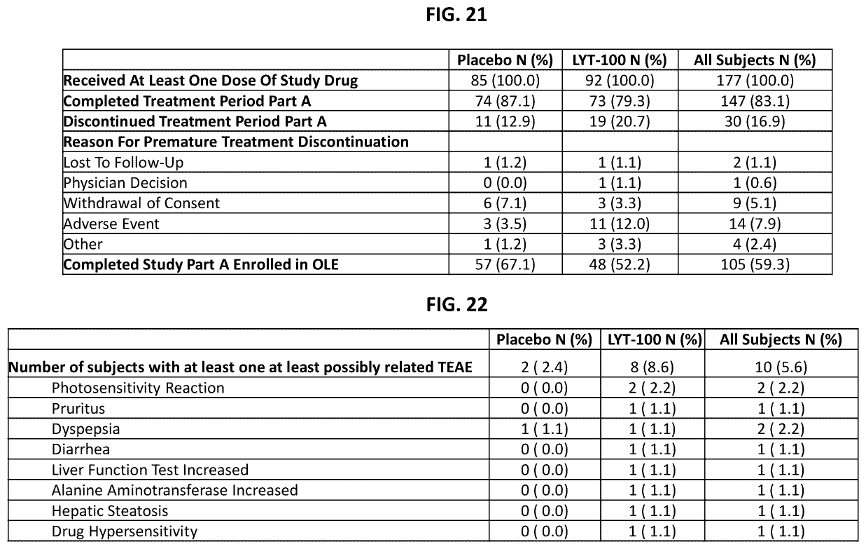

The study enrolled 177 patients averaging 55 years of age who experienced continued respiratory complications following hospitalization for acute COVID-19 infection that required treatment with supplemental oxygen were randomized to receive LYT-100 or placebo in a ratio of 1:1, respectively. The baseline demographic characteristics of enrolled subjects and subject disposition are provided in FIG. 19 to FIG. 21.

### Tolerability Results

LYT-100 was well-tolerated in this relatively sick patient population with multiple comorbidities and concomitant medications. There were no drug-related serious adverse events (SAEs) or deaths. The treatment emergent AE's occurring in the LYT-100 arm at a frequency greater than or equal to 5% are summarized in Table 20. With reference to Table 20, nausea was the only AE judged to be at least possibly related to LYT-100 with an incidence ≥5% (8.7% vs 2.4% with placebo). With further reference to Table 20, other AEs that have been commonly associated with pirfenidone and which were considered to be at least possibly related to LYT-100 treatment in this study included headache (4.3% vs. 1.2% with placebo), dizziness (3.3% vs. 1.2% with placebo), fatigue (2.2% vs. 0% with placebo), and rash (3.3% vs. 1.2% with placebo). Discontinuation rates due to AEs that were considered at least possibly related to LYT-100 were low in both arms (8.6% with LYT-100 vs. 2.4% with placebo) and the majority of discontinuations in the LYT-100 arm were due to idiosyncratic events and not AEs commonly associated with pirfenidone. A summary of all treatment emergent adverse events judged to at least possibly be related to LYT-100 are provided as FIG. 22.

TABLE 20
Treatment Emergent AEs occurring in LYT-100 (≥5%)

| | Placebo: N | LYT-100 750 mg |
|---|---|---|
| Adverse Event | (%) Events | BID: N (%) Events |
| Nausea | 2 (2.4) 2 | 8 (8.7) 10 |
| Dyspepsia | 2 (2.4) 2 | 6 (6.5) 6 |
| Nasopharyngitis | 1 (1.2) 1 | 6 (6.5) 7 |
| Upper abdominal pain | 2 (2.4) 2 | 5 (5.4) 6 |
| Increase in Fibrin D Dimer | 2 (2.4) 2 | 5 (5.4) 6 |
| Headache | 3 (3.5) 3 | 5 (5.4) 5 |

Overall, the results of this study with respect to safety and tolerability reaffirm the profile of strong safety and tolerability profile of LYT-100 observed in previous studies, including those described in Examples 1 and 2 herein. The safety and tolerability of the 750 mg BID dosage in this relatively sick patient population suggest it may be equally well tolerated in other patient populations, such as those with interstitial lung diseases or other fibrotic-mediated pulmonary-mediated diseases.

### Example 4: In Vitro Stability of Pirfenidone and LYT-100 in the Presence of Recombinant Human CYP Isozymes

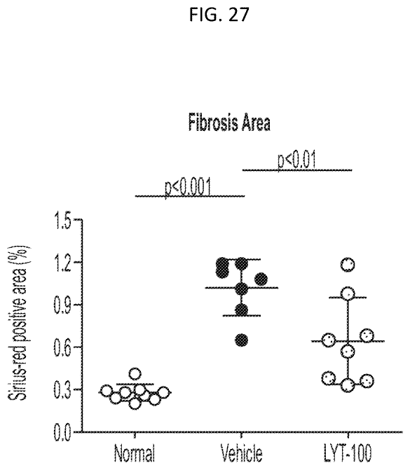

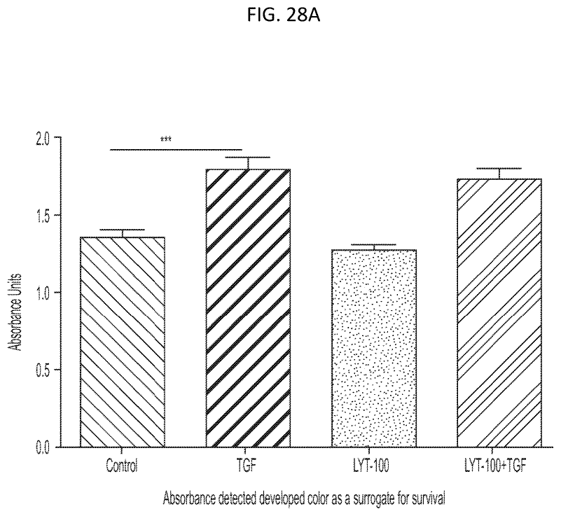

The metabolism of LYT-100 by isolated CYP isozyme preparations was evaluated and compared with the metabolism of pirfenidone (FIG. 27). Pirfenidone and LYT 100 were each incubated with recombinant human CYP1A2, CYP2C8, CYP2C9, CYP2C19, CYP2D6, CYP3A4, and CYP3A5 expressed in heterologous cell systems. The half-life (t1/2) of each test article was determined.

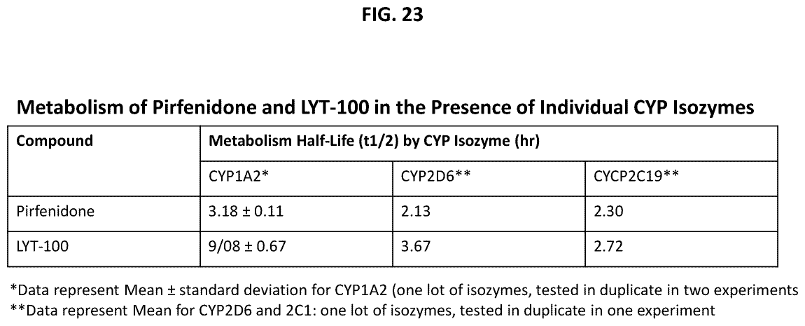

With reference to FIG. 23, pirfenidone and LYT-100 concentrations decreased by at least 15% during incubation with recombinantly expressed human CYP1A2, CYP2D6 and CYP2C19 isozymes. The t_{1/2} of pirfenidone following incubation with CYP1A2, CYP2C19 and CYP2D6 was 3.18, 2.13 and 2.30 hours, respectively. The t_{1/2} of LYT-100 following incubation with CYP1A2, CYP2C19 and CYP2D6 was 9.08, 3.67 and 2.72 hours, respectively. There was no significant metabolism by CYP2C8, CYP2C9, CYP3A4 or CYP3A5 isozymes, with more than 92% of the compounds remaining at the end of incubation. Therefore, no t_{1/2} was calculated for those isozymes. These results confirm the stabilization against metabolism of LYT-100 vs. pirfenidone. Metabolism by CYP1A2 was the most affected by deuteration (˜3-fold longer t_{1/2} compared to pirfenidone). This result demonstrates the effect of deuteration of LYT-100 on the overall metabolism of pirfenidone as the CYP1A2 isozyme plays a key role in the metabolism of pirfenidone.

### Example 5: Activity Screen

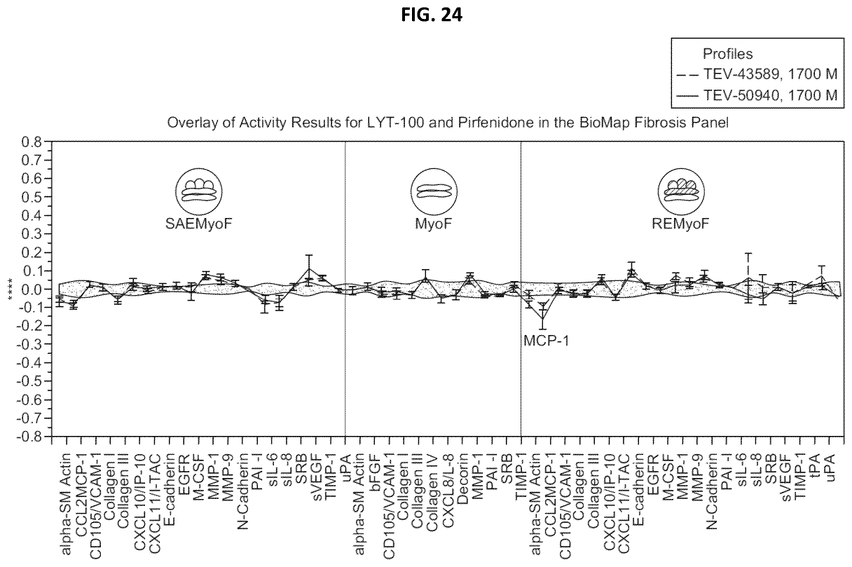

The DiscoverX BioMAP Fibrosis Panel was used to evaluate LYT-100 and pirfenidone. The panel contains 54 biomarker (cell surface receptors, cytokines, chemokines, matrix molecules and enzymes) readouts that capture biological changes that occur within the physiological context of the particular BioMAP system. LYT-100 and pirfenidone were tested in the BioMAP Fibrosis Panel at various dilutions starting at highest dose of 1700 μM in three cell/stimulus systems (myofibroblast [MyoF] composed of lung fibroblasts treated with TNF-α and TGF-β, renal proximal tubule epithelial cell (RE)MyoF including renal tubule epithelial cells and lung fibroblasts treated with TNF-α, and TGF-β, and small airway epithelial cell (SAE)MyoF comprising small airway epithelial cells and lung fibroblasts treated with TNF-a, and TGF-β). Similar results were observed with both compounds in the three systems (FIG. 24).

### Example 6: Inhibition of Lipopolysaccharide (LPS)-Induced Plasma TNF-α and IL-6 Concentrations Following Oral Dose of LYT-100 in Male Sprague-Dawley Rats

LYT-100 and pirfenidone dosing solutions were prepared by dissolving each in a vehicle of 1% carboxymethyl cellulose and 0.2% Tween-80 in water. LPS was diluted with sterile saline to 2 mg/mL and sonicated at 40° C. for 20 minutes to generate a stock solution and stored at 4° C. Each day of use, the stock solution was further diluted to 0.03 mg/mL in sterile saline.

Male Sprague-Dawley rats with indwelling femoral and jugular venous catheters (n=6-8 per dose and test article group) were used in this study. Rats were administered either vehicle (the dosing solution, 10 mL/kg), LYT-100 or pirfenidone at a concentration of 30, 100 or 300 mg/kg via syringe attached to an oral gavage needle. Sixty minutes after the oral dose, 0.03 mg/kg of LPS in 1 mL/kg saline was infused into the jugular vein.

Blood samples were collected from the femoral vein into heparin-coated syringes 15 minutes prior to the LPS infusion (45 minutes after oral doses of test article), 90 minutes after the LPS infusion and 4 hours after the LPS infusion. Plasma was prepared by centrifugation of the blood samples at 14,000 rpm for 10 minutes. Plasma samples were stored at −70° C. until analysis.

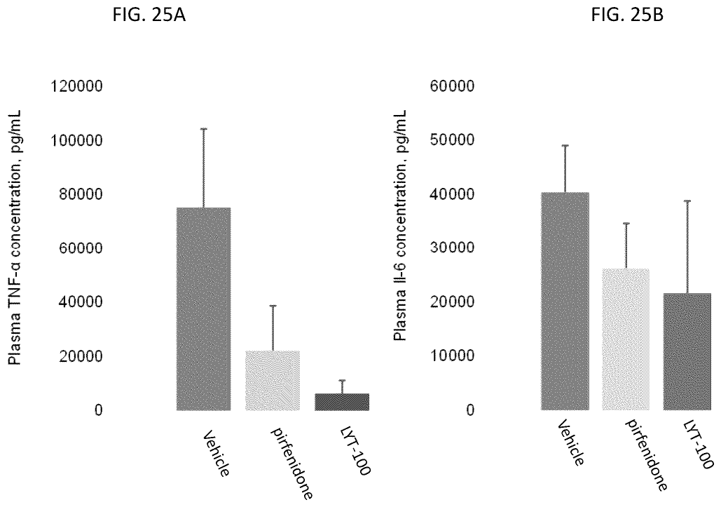

LPS stimulated a strong inflammatory response, including the cytokines TNF-α and IL-6 (FIGS. 25A and 25B, respectively). With reference to FIG. 25A, in vehicle-pretreated rats, LPS increased plasma TNF-α concentrations from non-detectable concentrations to approximately 75,000 μg/mL 90 minutes after injection). This TNF-α response to LPS was reduced by pretreatment with both pirfenidone and LYT-100. Ninety minutes after LPS injection, pretreatment with oral doses of 100 and 300 mg/kg LYT-100 inhibited TNFα. At 100 mg/kg, TNF-α levels were 70 percent lower than those obtained using equivalent oral volumes of the vehicle control, and there was greater reduction in TNF-α response in rats pretreated with LYT-100 compared to pirfenidone (Table 21, Table 22, and FIG. 25A). Pretreatment with oral doses of 100 and 300 mg/kg LYT-100 also inhibited IL-6 similarly to pirfenidone (Table 23, Table 24, and FIG. 25B). Thus, LYT-100 retains pirfenidone's activity to attenuate LPS-induced TNF-α and shows additional potency at an equivalent dose, likely due to the pharmacokinetic effect of deuteration.

TABLE 21
Plasma TNFα concentrations (pg/ml) after oral
pretreatment with vehicle, pirfenidone or LYT-100
and intravenous injection of LPS (1.5 hrs post-LPS)

| Dose, | Vehicle | Pirfenidone | LYT-100 |
|---|---|---|---|
| mg/kg | pre-treatment^{a} | pre-treatment^{a} | pre-treatment^{a} |
| 0 | 75389 ± 29107 | — | — |
| 30 | — | 46138 ± 20266 | 46382 ± 23254 |
| 100 | — | 22230 ± 16540 | 6403 ± 4812 |
| 300 | — | 11495 ± 10753 | 3162 ± 2668 |

^{a}Data represent Mean Standard Deviation; n = 6-8 per group

TABLE 22
Plasma TNFα concentrations (pg/ml) after oral
pretreatment with vehicle, pirfenidone or LYT-100
and intravenous injection of LPS (4 hrs post-LPS)

| Dose, | Vehicle | Pirfenidone | LYT-100 |
|---|---|---|---|
| mg/kg | pre-treatment^{a} | pre-treatment^{a} | pre-treatment^{a} |
| 0 | 693 ± 353 | — | — |
| 30 | — | 346 ± 83 | 274 ± 95 |
| 100 | — | 207 ± 92 | 129 ± 65 |
| 300 | — | 131 ± 32 | 79 ± 46 |

^{a}Data represent Mean Standard Deviation; n = 6-8 per group

TABLE 23
Plasma IL-6 concentrations after oral pretreatment
with vehicle, pirfenidone or LYT-100 and intravenous
injection of LPS (1.5 hrs post-LPS)

| Dose, | Vehicle | Pirfenidone | LYT-100 |
|---|---|---|---|
| mg/kg | pre-treatment^{a} | pre-treatment^{a} | pre-treatment^{a} |
| 0 | 40423 ± 8653 | — | — |
| 30 | — | 25173 ± 10651 | 18197 ± 6484 |
| 100 | — | 26226 ± 8434 | 21642 ± 17174 |
| 300 | — | 18692 ± 9996 | 13519 ± 8674 |

^{a}Data represent Mean Standard Deviation; n = 6-8 per group

TABLE 24
Plasma IL-6 concentrations after oral pretreatment
with vehicle, pirfenidone or LYT-100 and intravenous
injection of LPS (4 hrs post-LPS)

| Dose, | Vehicle | Pirfenidone | LYT-100 |
|---|---|---|---|
| mg/kg | pre-treatment^{a} | pre-treatment^{a} | pre-treatment^{a} |
| 0 | 13989 ± 6125 | — | — |
| 30 | — | 8886 ± 5321 | 8174 ± 5504 |
| 100 | — | 4603 ± 2524 | 4632 ± 5930 |
| 300 | — | 2361 ± 1185 | 1550 ± 1414 |

^{a}Data represent Mean Standard Deviation; n = 6-8 per group

### Example 7: LYT-100 Significantly Reduced Area of Fibrosis in Mouse Model

Non-alcoholic steatohepatitis (NASH) is characterized by lobular inflammation, hepatocyte ballooning and degeneration progressing to liver fibrosis. LYT-100 was orally administered at 0 mL/kg (Vehicle only: 0.5% carboxymethylcellulose) or 10 mL/kg twice daily from 6-9 weeks of age in 18 male mice in which NASH mice was induced by a single subcutaneous injection of 200 μg streptozotocin solution 2 days after birth and feet with a high fat diet after 4 weeks of age. LYT-100 was administered at an oral dose of 30 mg/kg twice daily (60 mg/kg/day). In addition, nine non-NASH mice were fed with a normal diet and monitored.

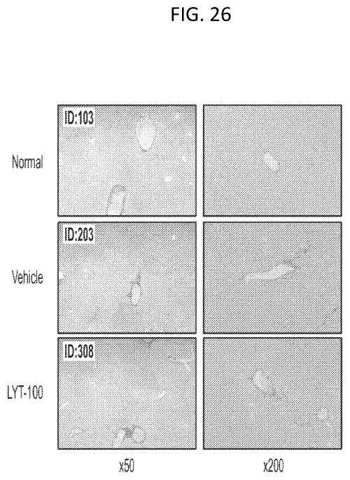

FIG. 26 depicts representative micrographs of Sirius-red stained liver sections illustrating that LYT-100 significantly reduced the area of fibrosis. Specifically, liver sections from the vehicle group exhibited collagen deposition in the pericentral region of the liver lobule. Further, the LYT-100 group showed a significant reduction in the fibrosis area compared to the vehicle group. These results demonstrate that LYT-100 has a potential to inhibit the progression of fibrosis. FIG. 27 illustrates the percent fibrosis area for LYT-100 versus vehicle and control. The results are also summarized Table 25 below.

TABLE 25
Fibrosis Area

| Parameter | Normal | Vehicle | LYT-100 |
|---|---|---|---|
| (mean ± SD) | (n = 9) | (n = 7) | (n = 8) |
| Fibrosis Area (%) | 0.27 ± 0.06 | 1.02 ± 0.20 | 0.64 ± 0.31* |

*p < 0.01, Vehicle vs LYT-100

Liver sections from the Vehicle group exhibited severe micro- and macro vesicular fat deposition, hepatocellular ballooning and inflammatory cell infiltration. While LYT-100 hepatocyte ballooning was similar to Vehicle, scores were lower for lobular inflammation and steatosis (Table 26). The components of the NAS Score are provided in Table 27.

TABLE 26
NAFLD Activity Score

| | | Score | | | | | | | | | | | |
|---|---|---|---|---|---|---|---|---|---|---|---|---|---|
| | | | | | | | | | | Hepatocyte | | | NAS |
| | | Steatosis | | | | Lobular Inflammation | | | | ballooning | | | (mean ± |
| Group | n | 0 | 1 | 2 | 3 | 0 | 1 | 2 | 3 | 0 | 1 | 2 | SD) |
| Normal | 9 | 9 | — | — | — | 9 | — | — | — | 9 | — | — | 0.0 ± |
| | | | | | | | | | | | | | 0.0 |
| Vehicle | 7 | 2 | 5 | — | — | — | 2 | 1 | 4 | — | 1 | 6 | 4.9 ± |
| | | | | | | | | | | | | | 1.2 |
| LYT- | 8 | 4 | 3 | 1 | — | — | 5 | 3 | — | — | — | 8 | 4.0 ± |
| 100 | | | | | | | | | | | | | 1.1 |

TABLE 27
Definitions of NAS Components

| Item | Score | Extent |
|---|---|---|
| Steatosis | 0 | <5% |
| | 1 | 5-33% |
| | 2 | >33%-66% |
| | 3 | >66% |
| Hepatocyte | 0 | None |
| Ballooning | 1 | Few balloon cells |
| | 2 | Many cells/prominent ballooning |
| Lobular | 0 | No foci |
| Inflammation | 1 | <2 foci/200x |
| | 2 | 2-4 foci/200x |
| | 3 | >4 foci/200x |

As evidenced above, LYT-100 significantly reduced the area of fibrosis, reduced inflammation, and reduced accumulation of fat (steatosis), as compared to the untreated NASH mice.

### Example 8: LYT-100 Reduction of TGF-β-Induced Proliferation and Collagen Levels in Primary Mouse Lung Fibroblasts

LYT-100 was evaluated for an ability to reduce the TGF-β-induced proliferation of, and collagen levels in, Primary Mouse Lung Fibroblasts (PMLF).

Inhibition of p38 members by LYT-100 is important as p38 members are activated by the TGF-β signaling pathway. TGF-β activation in turn plays a significant role in transcriptional induction of the collagen type IA2. The collagen type IA2 makes up the majority of extracellular matrix, which accumulates during progression of, e.g., fibrotic lung disease. Deposition of collagen is one of the most important components of fibrotic lung tissue, a process primarily induced by TGF-β. Since accumulation of insoluble collagen encroaches on the alveolar space, it plays pivotal role in distortion of lung architecture and progression of fibrotic lung disease. In addition to insoluble (structural) collagen, fibrotic lungs of IPF patients also show high levels of non-structural (soluble) collagen. Although this type of collagen may eventually become insoluble collagen, until then, soluble collagen can serve as a ligand for integrin receptors of lung fibroblasts and epithelial cells. Binding of soluble collagen to these receptors induces proliferation and migration of these cells. Fibronectin is another important component of fibrotic lungs as it is induced by TGF-β and functions both as a structural component of extra cellular matrix (ECM), as well as a ligand for integrin receptors. Just like soluble collagen, binding of fibronectin to integrin receptors induces the proliferation of fibroblast and epithelial cells of the lungs and plays a significant role in progression of IPF. Therefore, inhibition of TGF-β-induced collagen synthesis is an important target for fibrotic lung disease.

### Preparation of Primary Mouse Lung Fibroblast

Primary Mouse lung fibroblast were prepared as follows. One lung was removed from 2 months old male BalbC Mouse, perfused with sterile PBS, minced and incubated in 2 ml of serum free Dulbecco's Modified Eagle's Medium (DMEM) containing 100 μg/ml of collagenase I for one hour at 37° C. Each sample was centrifuged at 1500 r.p.m (revolution per minute) for 5 minutes, washed three times with PBS and the final cellular pellet was resuspended in DMEM supplemented with 10% serum and Pen/Strep, and incubated in 150 mm plates at 37° C. with 80% humidity and %% CO₂. The growth medium was removed and fresh medium was added every day for 10 days.

### Testing the Effect of LYT-100 on Survival of Primary Mouse Lung Fibroblast

LYT-100 was evaluated for an ability to alter TGF-β-induced proliferation of PMLF. At the end of 10-day incubation period above, lung fibroblasts were confluent. Before testing the effect of LYT-100 on survival of these cells, fibroblasts were tripsinized and five thousand cells were plated into 96 well plate in 200 μL complete DMEM, and incubated until cells reached to 95-100% confluency, then the medium was removed and complete DMEM containing proline (10 μM) and ascorbic acid (20 μg/ml) was added. LYT-100, dissolved in pure ethanol, was added to the plates at a final concentration of 500 μM 1 h prior to addition of TGF-β (5 ng/ml), and cells were further incubated for 72 hrs. One hundred μL of the growth medium was removed and 20 μL of MTT stock solution (prepared in PBS at 5.5 mg/ml concentration) was added and cells were incubated for 4 hours, then 100 μl of dimethyl sulfoxide was added, and absorbance of developed color was monitored at 540-690 nm.

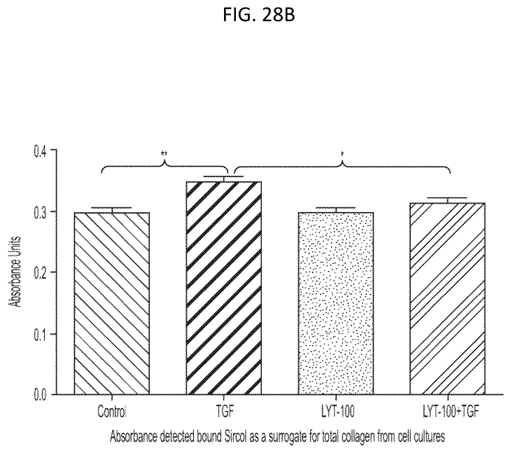

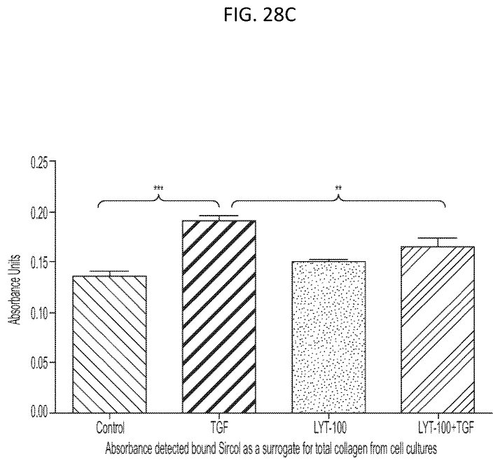

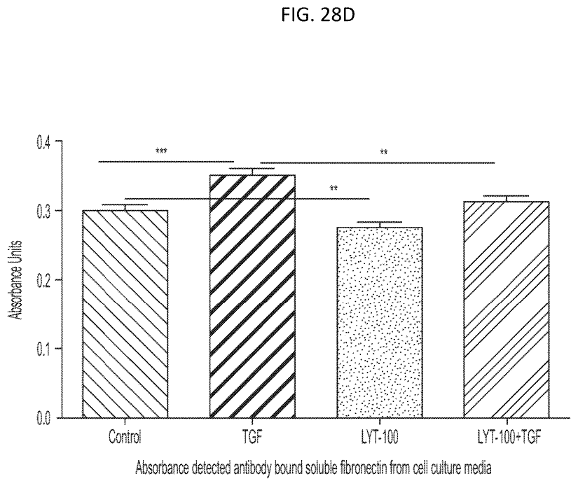

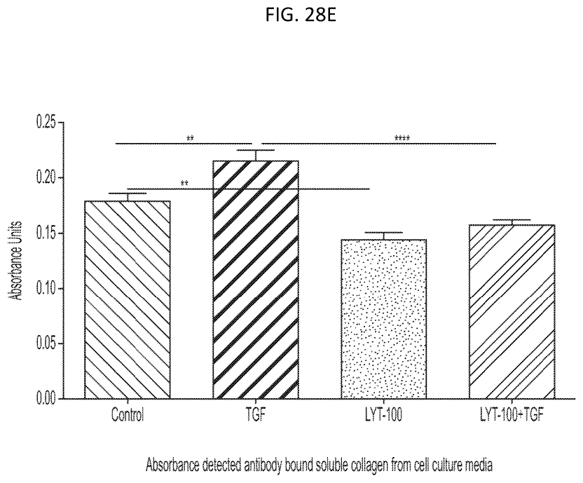

As shown in FIG. 28A, LYT-100 did not affect the survival of PMLFs alone. TGF-β (5 ng/ml) significantly induced the proliferation of PMLFs by nearly 45% (p=0.001), and LYT-100 did appear to diminish TGF-β-induced proliferation of PMLFs by 10%, but this effect was not statistically significant (p=0.19).

### TGF-β-Induced Insoluble Collagen Synthesis Using 6-Well Plate Format

The effect of LYT-100 on inhibition of TGF-β-induced collagen synthesis was evaluated in PMLFs in a 6-well format. One hundred thousand Primary Mouse Lung Fibroblasts were plated in 6-well plates and incubated in complete DMEM until they reached confluency. The incubation medium was removed and complete DMEM containing proline (10 μM) and ascorbic acid (20 μg/ml) was added. LYT-100 was added to the plates at a final concentration of 500 μM 1 h prior addition of TGF-β (5 ng/ml), and cells were further incubated for 72 hrs.

Supernatant was removed, cells were washed with cold PBS and 1 ml Sircol reagent was added. The Sircol reagent contains the collagen binding dye Sirius red. The cells were scraped off with Sircol reagent and samples were shaken for 5 h at room temperature (RT), centrifuged at 10,000 rpm for 5 min, supernatant was removed, the pellet was washed in 0.5 M acetic acid to remove unbound dye, and recentrifuged at 10,000 rpm for 5 min, supernatant was removed, and the final pellet was dissolved in 1 ml 0.5M NaOH and shaken at RT for 5 h. A sample of 100 μl of resultant solution was placed in 96-well. The color reaction was assessed by optical density at a wavelength of 600 nm.

As shown in FIG. 28B, PMLFs responded to TGF-β with increased total collagen levels, (increase of 21%; p=0.0087). LYT-100 inhibited this induction by 15% (p=0.026), as compared to the TGF-β alone, without reducing the background level of collagen.

### TGF-β-Induced Insoluble Collagen Synthesis Using 96-Well Plate Format

The effect of LYT-100 on TGF-β-induced collagen was confirmed in a high throughput collagen assay using 96-well plate format. Approximately 5,000 primary mouse fibroblasts were plated in complete DMEM in 96 well plates and incubated for 3 days at which time the cultures achieved confluency. After cells reached confluency, the medium was removed and fresh DMEM supplemented with ascorbic acid (20 μg/ml) and prolin (10 μMol) was added. LYT-100 was then added to the appropriate cultures at a final incubation concentration of 500 μM. One hour later, TGF-β was added to the appropriate cultures at a final concentration of 5 ng/ml. After 72 hours, the media was replaced with a 0.5% glutaraldehyde solution. After 30 minutes, the adherent cells were washed and subsequently incubated with acetic acid at a final concentration of 0.5M. After a 30 min room temperature incubation, and subsequent washing steps, the wells were incubated with Sircol reagent. After 5 hours, the unbound dye was removed and the plates were washed and allowed to dry. To extract collagen-bound Sircol, 100 μL of alkaline solution (0.5M NaOH) was added and plates were shaken for 1 h on rotary shaker at room temperature. Absorbance at 600 nm was determined to detect bound collagen.

As shown in FIG. 28C, in the 6-well format, TGF-β induced insoluble collagen level by 40% (p=0.0002), LYT-100 diminished this TGF-β-stimulated collagen accumulation by 24% (p=0.0003) without reducing the background level of collagen.

### TGF-β-Induced Soluble Fibronectin and Collagen Synthesis

LYT-100 was evaluated for its ability to modify TGF-β-induced soluble fibronectin and soluble collagen synthesis using a selective ELISA. Approximately 5,000 primary mouse lung fibroblasts were plated in complete DMEM in 96 well plates and incubated for 3 days at which time the cultures achieved confluency. After cells reached to confluency, medium was removed and fresh DMEM supplemented with ascorbic acid (20 μg/ml) and prolin (10 μM) was added. LYT-100 was then added to the appropriate cultures at a final incubation concentration of 500 μM. One hour later, TGF-β (5 ng/ml) was added to the appropriate cultures at a final concentration. After 72 hours, 200 μl samples of the supernatant were placed onto an ELISA plate and incubated overnight. After blocking with %1 BSA for 2 h, plates were incubated with either an anti-collagen type I antibody or an anti-fibronectin antibody.

The plates were washed after 1 hour and incubated with secondary horseradish peroxidase-conjugated antibodies (anti-goat for the collagen antibody, anti-rabbit for the fibronectin antibody). After a series of washing steps the color reagent TMB (3,3′,5,5′-tetramethylbenzidine) was added and 15 minutes later the reactions were terminated with equal volumes of 2N H₂SO₄. The levels of soluble collagen and fibronectin were determined by evaluating absorbance at 450 nm.

Referring to FIG. 28D, TGF-β induced the level of soluble fibronectin by 16% (p=0.0021). LYT-100 inhibited TGF-β-dependent induction of fibronectin by 11% (p=0.0185). Moreover, LYT-100 also inhibited the background level of soluble fibronectin by 10% (p=0.03).

As shown in FIG. 28E, TGF-β induced the level of soluble collagen by 20% (p=0.0185). LYT-100 inhibited this TGF-β-dependent increase by 36% (p=0.0001). Moreover, it also inhibited background level of soluble collagen by 23% (p=0.0115).

In summary, LYT-100 was found to: (i) reduce TGF-β-induced cell proliferation, (ii) reduce both background and TGF-β-induced levels of insoluble (structural) collagen; (iii) reduce both background and TGF-β-induced levels of soluble collagen; and (iv) reduce both background and TGF-β-induced levels of soluble fibronectin.

During the progression of fibrotic lung diseases such as IPF, an accumulation of extra cellular matrix components such as collagen and an increase in the fibroblast population is observed. Persistent proliferation of fibroblasts is considered an important contributor to the lung architecture in fibrotic lung disease, including the diminished interstitial spaces of the alveoli. Thus, reducing TGF-β-induced proliferation of fibroblasts and structural collagen with LYT-100 has the potential to prolong lung function in fibrotic lung disease. In addition to inhibiting TGF-β-induced insoluble collagen level, LYT-100 also inhibits TGF-β-induced secreted collagen and fibronectin 3. Secreted collagen and fibronectin not only increase the rate of formation of fibrotic foci in the lung, but these proteins can also act as ligands for integrin receptors. When integrin receptors are activated, they induce not only the proliferation of epithelial cells and fibroblasts of the lungs, but they also, along with TGF-β, induce epithelial mesenchymal transition (EMT) of the epithelial cells of the lungs. EMT causes these cells to migrate to different regions of the lungs. This migration is considered to be a very important contributor for the generation of new fibrotic foci in the lungs and progression of fibrotic lung disease such as IPF. LYT-100 has the ability to inhibit TGF-β-induced pro-fibrotic processes and to reduce basal factors which have the potential to exacerbate ongoing fibrosis.

### Example 9: Effect of LYT-100 on L929 Cells

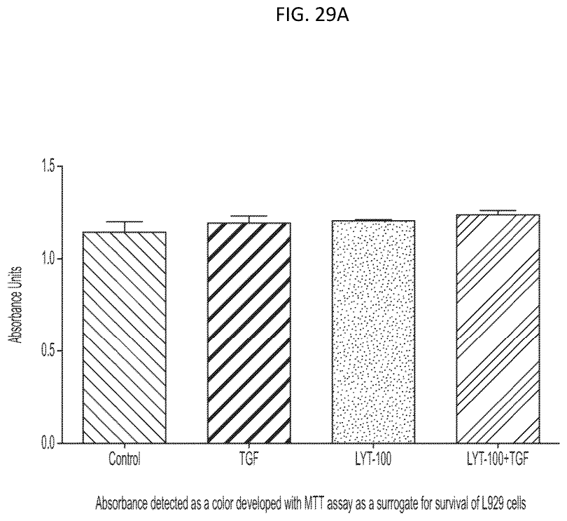

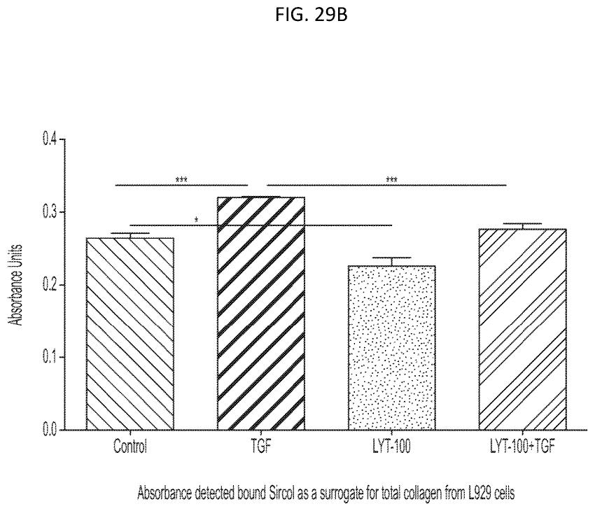

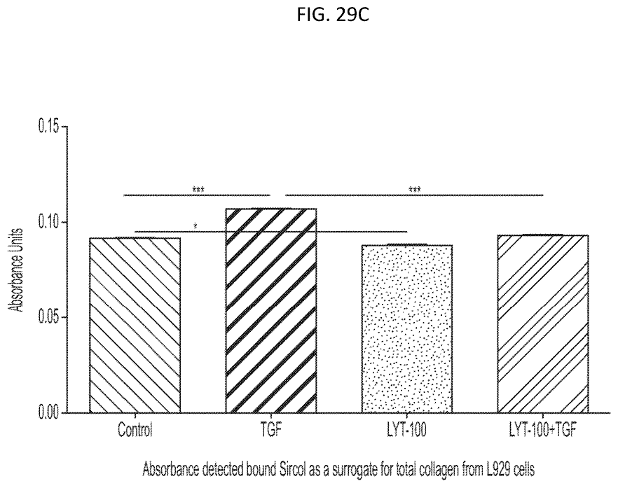

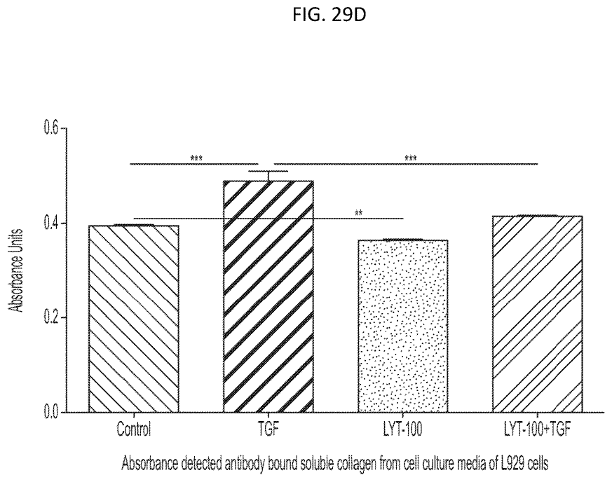

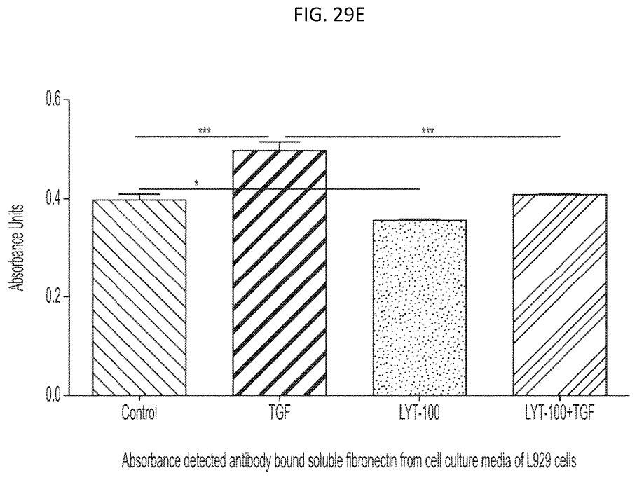

The effect of LYT-100 on survival of L929 cells was determined. Five thousand L929 cells were plated in completed DMEN and incubated until confluency for 3 days. The medium was removed and complete DMEM containing proline (20 μg/ml) and ascorbic acid (10 uM) was added. LYT-100 was given at 500 μM 1 h prior addition of TGFβ (5 ng/ml), and cells were further incubated for 72 hrs. An aliquot of 100 μL of medium was removed, 20 μL MTT solution was added for 4 hrs, then 100 μl of DMSO was added, and absorbance of the developed dark pink color was determined at 54-690 nM. FIG. 29A illustrates that LYT-100 does not affect survival of L929 cells.

The effect of LYT-100 on TGF-induced collagen synthesis in 6-wells was determined. 100,000 L929 cells were plated in complete DMEN and incubated until confluency for 3 days. Medium was removed and complete DMEM containing proline (20 μg/ml) and ascorbic acid (10 μM) was added. LYT-100 was added at 500 μM 1 hour prior addition of TGF-β (5 ng/ml). Cells were further incubated for 72 hrs. Supernatant was removed, cells were washed with cold PBS, 1 ml Sircol reagent was added onto the cells and cells were scraped off, samples were shaken for 5 h at RT, centrifuged at 10,000 rpm for 5 min, supernatant was removed, the pellet was dissolved in 0.5M acetic acid to remove unbound dye, and re-centrifuged at 10,000 rpm for 5 min, supernatant was removed and the final pellet was dissolved in 1 ml of 0.5M NaOH, shaken at RT for 5 h, 100 μl of resulted solution was placed in 96-well and absorbance was determined at 600 nM. The results are summarized in FIG. 29B, which illustrates that LYT-100 inhibits TGF-induced collagen synthesis. LYT-100 also significantly inhibits collagen synthesis in the absence of added TGF-β.

Next, the effect of LYT-100 on TGF-induced collagen synthesis was confimed using a 96-well plate format. Five thousand L929 cells were plated in complete DMEN and incubated until confluency for 3 days. The medium was removed and complete DMEM containing proline (20 μg/ml) and ascorbic acid (10 μM) was added. LYT-100 was added at 500 μM 1 h prior addition of TGF-β (5 ng/ml). Cells were further incubated for 72 hrs. Supernatant was removed, 0.5% gluteraldehyde was added for 30 min at RT, removed, washed 3× with water, 0.5M acetic acid was added for 30 min at RT, removed, washed with water, air dried and 100 μl Sircol dye was added for 5 h at RT. The dye was removed, the plate was washed extensively under running water, air dried, and 200 μl of 0.5M NaOH was added, the plates were shaken at RT for 1 h, and absorbance was determined at 600 nm. The results summarized in FIG. 29C illustrate that LYT-100 significantly inhibited or reduced TGF-β-induced total collagen levels. LYT-100 also significantly inhibited or reduced total collagen level in the absence of TGF-β induction.

The effect of LYT-100 on TGF-induced soluble collagen synthesis was determined using a 96-well plate format. Five thousand L929 cells were plated in complete DMEN and incubated until confluency for 3 days. The medium was removed and complete DMEM containing proline (20 μg/ml) and ascorbic acid (10 μM) was added. LYT-100 was added at 500 μM 1 h prior addition of TGF-β (5 ng/ml). Cells were further incubated for 72 hrs. 200 μl supernatant of 96-well Sircol plate was placed onto ELISA plate and incubated overnight. The next day, supernatant was removed and 100 ul of 1% BSA in PBST was added and incubated for 2 h at RT, BSA was removed, plate was washed 3× with 200 μl of PBST, and anti-collagen type I a.b was added at 1:2000 dilution (prepared in %1 BSA in PBST), incubated at RT for 1 h, primary a.b was removed, plate was washed 3× with 200 μl PBST, and secondary anti-goat HRP was added at 1:2000 dilution, incubate at room temperature for 1 h, removed, the plate was washed 3× with 200 μl PBST, and 100 μl of TMB solution was added for color development for 15 min, then 100 μl of 2N H2SO4 was added to stop the reaction and absorbance of the developed yellow color was determined at 450 nm.

As illustrated in FIG. 29D, LYT-100 significantly inhibits TGF-β-induced soluble collagen levels. LYT-100 also significantly reduced soluble collagen levels in the absence of TGF-β-induction.

Fibronectin is another important component of fibrotic lungs as it is induced by TGF-β and functions both as a structural component of extra cellular matrix as well as well as a ligand for integrin receptors. Just like soluble collagen, binding of fibronectin to integrin receptors induces the proliferation of fibroblast and epithelial cells of the lungs. The effect of LYT-100 on TGF-induced soluble fibronectin synthesis was determined using a process similar to that described in the above paragraph for soluble collagen synthesis, except that a fibronectin ELISA was used. As illustrated in FIG. 29E, LYT-100 significantly reduced soluble fibronectin levels, in the absence and presence of TGF-β-induction.

### Example 10: LYT-100 Study in Mouse Model of Lymphedema

This experiment tested the effect of LYT-100 in a mouse tail model of lymphedema. LYT-100 or control (carboxymethylcellose) was delivered once daily by oral gavage, in mice with ablated tail lymphatics via circumferential excision and ablation of collecting lymphatic trunks. Tail volume was measured weekly for all animals, starting pre-surgery and continuing until the occurrence of COVID19 required termination of the study at 6 weeks. At sacrifice, tails were harvested for histology and immunofluorescent imaging to characterize tissue changes with surgery and LYT-100 or control treatment. Tail volume and markers of lymphatics, fibrosis, and inflammation were compared between LYT-100 and the control group.

Animals: 14 adult (10-14 week old) C57BL/6 J mice. 7 animals per group.

Surgery: The superficial and deep collecting lymphatics of the mid portion of the tail were excised using a 2-mm full-thickness skin and subcutaneous excision performed at a distance of 15 mm from the base of the tail. Lymphatic trunks (collecting lymphatics) adjacent to the lateral veins were identified and ablated through controlled, limited cautery application under a surgical microscope.—The dosing amounts, route and schedule are provided in Table 28.

TABLE 28
Dosing regimens

| | | | | Dosing |
|---|---|---|---|---|
| | Test | Test article | | route and |
| Group | article | preparation | Dosing | schedule |
| Group 1 | LYT-100 | Crystals ground into | 250 mg/kg/day | Oral |
| | | fine powder and | | gavage, |
| | | suspended in 0.5% | | twice |
| | | carboxymethy- | | daily |
| | | cellulose (40 mg/mL) | | |
| Group 2 | LYT-101 | Crystals ground into | 250 mg/kg/day | Oral |
| | | fine powder and | | gavage, |
| | | suspended in 0.5% | | twice |
| | | carboxymethy- | | daily |
| | | cellulose (40 mg/mL) | | |
| Group 3 | Control | 0.5% carboxymethy- | 10 mL/kg/day | Oral |
| | | cellulose | | gavage, |
| | | | | twice |
| | | | | daily |
| Group 4 | LYT-100 | Crystals ground into | 250 mg/kg/day | Oral |
| | | fine powder and | | gavage, |
| | | suspended in 0.5% | | twice |
| | | carboxymethy- | | daily |
| | | cellulose (40 mg/mL) | | |
| Group 5 | LYT-101 | Crystals ground into | 250 mg/kg/day | Oral |
| | | fine powder and | | gavage, |
| | | suspended in 0.5% | | twice |
| | | carboxymethy- | | daily |
| | | cellulose (40 mg/mL) | | |
| Group 6 | Control | 0.5% carboxymethy- | 10 mL/kg/day | Oral |
| | | cellulose | | gavage, |
| | | | | twice |
| | | | | daily |

Measurements are provided in Table 29.

TABLE 29
Measurements

| | |
|---|---|
| Tail volume | Calculated with truncated cone formula (Sitzia 1995) and confirmed |
| | using histological measurements of soft tissue thickness of the |
| | skin/subcutaneous tissues was measured serially using digital images |
| | of histology slides stained with hematoxylin and eosin |
| Histology | Tissues fixed in 4% paraformaldehyde at 4° C., decalcified in 5% |
| | sodium EDTA (Santa Cruz Biotechnology, Dallas, Tex.), embedded |
| | in paraffin, and sectioned at 5 micrometers. Cut sections rehydrated |
| | and heat-mediated antigen unmasking performed using 90° C. sodium |
| | citrate (Sigma-Aldrich). Non-specific binding blocked with 2% |
| | BSA/20% animal serum. Tissues incubated overnight with primary |
| | antibody at 4° C. Primary antibodies used for immunohistochemical |
| | stains include goat anti-mouse LYVE-1, rat anti-mouse CD45, rabbit |
| | anti-mouse CD4, Cy3-conjugated mouse anti-αSMA (from Sigma- |
| | Aldrich), rabbit anti-human IFN-γ, rabbit anti-mouse TGF-β1, rabbit |
| | anti-mouse p-SMAD3, rabbit anti-mouse collagen I (all from |
| | ABCAM, Cambridge, MA) |
| Immunofluorescence | Immunofluorescence staining performed with AlexaFluor |
| imaging | fluorophore-conjugated secondary antibodies (Life Technologies, |
| | Norwalk, CT). Images scanned using Mirax imaging software (Carl |
| | Zeiss). Peri-lymphatic CD45+ and CD4+ cell counts assessed by |
| | counting positively stained cells within 50 μm of the most inflamed |
| | lymphatic vessel in each quadrant of the leg. Positively stained cells |
| | counted by two blinded reviewers in four randomly-selected, 40× |
| | high-power fields in a minimum of 4 fields per animal. Collagen I |
| | deposition quantified using Metamorph software (Molecular Devices, |
| | Sunnyvale, CA) in dermal areas of 5 μm cross-sections. This analysis |
| | confirmed using picrosirius red staining (Polysciences, Warrington, |
| | PA) using manufacturer's instructions. Scar index quantified with |
| | Metamorph software by calculating the ratio of red-orange:green- |
| | yellow fibers with higher numbers representing increased scarring. |

Study procedure and timing are provided in Table 30.

TABLE 30
Study Details

| Time | | Procedure | Notes |
|---|---|---|---|
| 0 | weeks | Surgery | Lymphatic tail |
| | | | surgery |
| 6 | weeks | Begin intervention (daily oral gavage) | |
| 12 | weeks | Interim sacrifice groups | |
| 18 | weeks | Late sacrifice groups | |

| Weekly | Tail volume measurement | From pre-surgery |
|---|---|---|
| | Statistical Analysis | ANOVA |

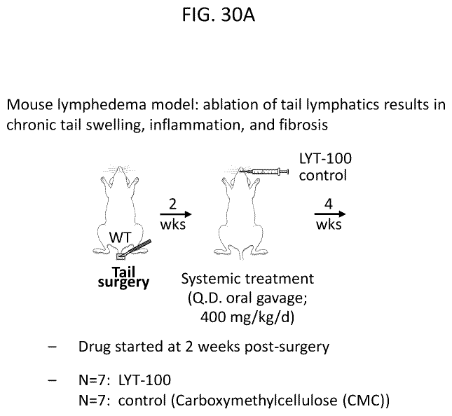

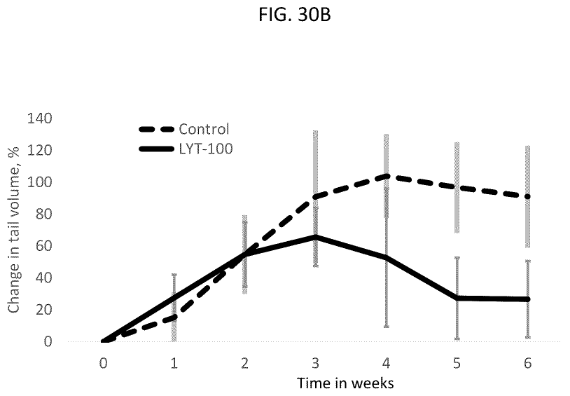

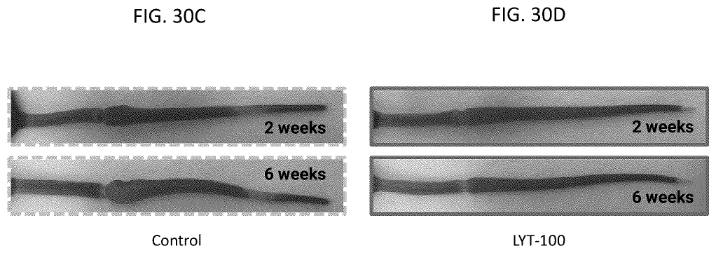

FIG. 30A-D depicts results of once daily administration of LYT-100 to reduce swelling in a mouse lymphedema model over the six weeks. The mouse lymphedema model is graphically illustrated in FIG. 30A. As shown in FIG. 30B, daily administration of LYT-100 significantly reduced swelling as compared to carboxymethylcellulose control by 5 weeks. The images in FIG. 30C and FIG. 30D depict the differences in swelling at 6 weeks.

### Example 11: Evaluation of LYT-100 Efficacy in a Rodent Bleomycin-Induced Fibrosis Model

The rodent bleomycin-induced fibrosis model (BLM) is commonly utilized in the preclinical setting as it appears to have clinical relevance as an animal model of human fibrosis (e.g., idiopathic pulmonary fibrosis) based on the observed pulmonary pathophysiology following the bleomycin challenge in rats. See, e.g., Corboz et al., Pumonary Pharm. & Ther. 49 (2018), 95-103). Bleomycin is a metabolite of the bacterium Streptomyces verticillus first identified in 1962. Specifically, bleomycin is a non-ribosomal hybrid peptide-polyketide natural product having the structure:

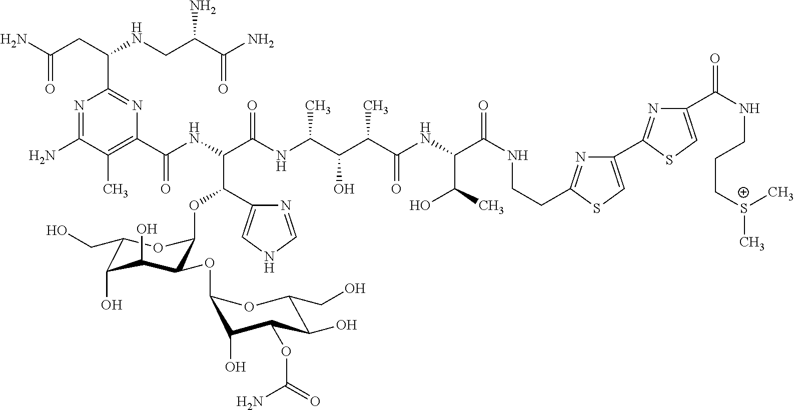

While bleomycin possesses antibacterial activity, its toxicity precludes use as an antibiotic. Bleomycin is used as a chemotherapeutic agent in the treatment of various cancers, including Hodgkin's lymphoma, non-Hodgkin's lymphoma, testicular cancer, ovarian cancer, and cervical cancer among others. Bleomycin acts by induction of DNA strand breaks and may also inhibit incorporation of thymidine into DNA strands. DNA cleavage by bleomycin depends on oxygen and metal ions, at least in vitro, though the exact mechanism of DNA strand scission is unresolved.

Common side effects associated with bleomycin chemotherapy include fever, weight loss, vomiting, rash, and a severe type of anaphylaxis. The most serious complication of bleomycin therapy, occurring with increasing dosage, is pulmonary fibrosis and impaired lung function. In high concentrations, bleomycin induces DNA strand rupture, generates free radicals, and causes oxidative stress tresulting in cell necrosis and/or apoptosis. Recent studies support the role of the proinflammatory cytokines IL-18 and IL-1beta in the mechanism of bleomycin-induced lung injury. Bleomycin is normally metabolized by the enzyme bleomycin hydrolase, but the lung is particularly susceptible to bleomycin toxicity by virtue of the scarcity of this enzyme in the lung. Lung inflammation, fibrosis, reductions in lung compliance, and impaired gas exchange are the consequences of a bleomycin challenge.

In assessing anti-fibrotic potential of compounds of interest, evaluation is generally performed in the phase of established fibrosis, i.e., 10-15 days after the initiation, rather than in the early period of bleomycin-induced inflammation. Conversion of proline into hydroxyproline and incorporation into lung collagen occurs as early as 4 days after bleomycin administration. The switch between inflammation and fibrosis occurs in rats around day 9 after bleomycin administration. It was deemed desirable to evaluate activity of LYT-100 during both the inflammatory and fibrotic stages of the model. Accordingly, LYT-100 was administered starting at day 8 following bleomycin administration.

### Phase I Study

Initially, a Phase I study was conducted to evaluate the effect of bleomycin and LYT-100 on body weight and lung weight in the rat BLM induced lung fibrosis model. The Phase I study design is provided in Table 31.

TABLE 31
BLM Study Design- Phase I

| | | | Test Article | | Day 14 |
|---|---|---|---|---|---|
| | Intervention | Test Article | Dosing | Number of | Necropsy |
| Group | (days 1-7) | and Dose | (Day 8-13) | Animals | and analysis |
| 1 | Saline; Days | LYT-100 | PO; oral gavage | N = 3 | Lung weights, |
| | 1, 2, 3, 6, | (400 mg/kg) | as solution in | | Lungs inflation |
| | and 7 | | 1% aq. CMC; | | (fixed with 10% |
| 2 | Bleomycin | LYT-100 | QD | N = 4 | NBF overnight, |
| | 0.45 mg/kg; | (250 mg/kg) | | | then kept in |
| | Days 1, 2, 3, | | | | 70% ethanol) |
| | 6, and 7 | | | | |
| 3 | Bleomycin | LYT-100 | | N = 4 | |
| | 0.45 mg/kg; | (400 mg/kg) | | | |
| | Days 1, 2, 3, | | | | |
| | 6, and 7 | | | | |

For Groups 1, 2 and 3, bleomycin and vehicle dosing were conducted as indicated in Table 31 (0.45 mg/kg, at 1696 IU/mg of Bleomycin or saline on Day 1, 2, 3, 6 and 7). On days 8 to 13, LYT-100 was dosed via oral gavage once daily.

### Observations

Animals were observed for a variety of clinical signs and symptoms following bleomycin and LYT-100 dosing. All animals dosed with bleomycin or saline had 100% incidence of abnormal sounds on Days 1, 2, 3, 6 and 7 which was alleviated by the next study day, confirming dosing to the lung. All animals dosed with bleomycin (Group 2 and 3) were observed with respiratory signs from Day 3, with 100% incidence of increased respiratory rate by Day 5. There was no observed increased respiratory rate for Group 1. Respiratory signs are an indication of acute inflammation secondary to bleomycin challenge. Some animals were observed with abnormal gait following initiation of LYT-100 administration on Day 8. The sign disappeared from the animals that showed it ˜5 h after it was recorded, and it did not appear in the subsequent dosing occasions. Almost all the animals were noted to be subdued and with decreased activity following LYT-100 dosing on Days 8, 9 and 10, after which point the sign appeared only in Group 3 (Bleomycin/400 mg/kg LYT-100) on Day 13. When this signa appeared, it disappeared ˜5 h after it was recorded. All animals were observed with eyelids closed following initiation of LYT-100 administration on Day 8. The sign disappeared from the animals that showed it ˜5 h after it was recorded, and it did not appear in the subsequent dosing occasions. Some animals in Groups 1 and 2 were observed with erected fur following initiation of LYT-100 administration on Day 8 and again on Day 11. The sign disappeared from the animals that showed it ˜5 h after it was recorded, and it did not appear in the subsequent dosing occasions. Almost all of the animals were observed salivating following initiation of LYT-100 administration on Day. The sign disappeared from the animals that showed it ˜5 h after it was recorded, and it did not appear in the subsequent dosing occasions.

### Results

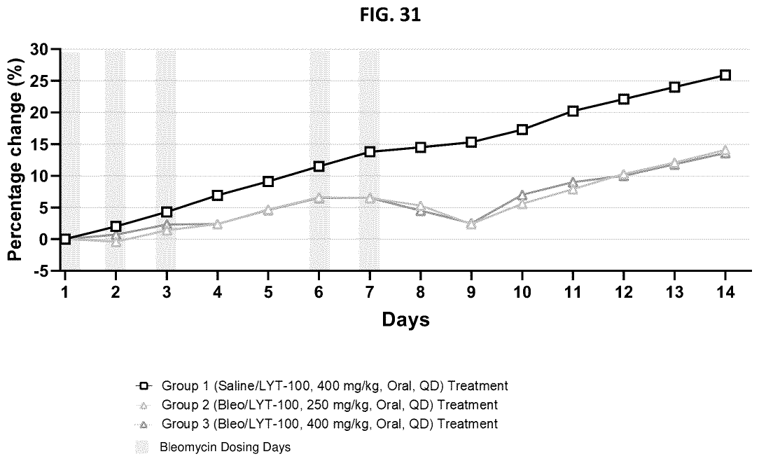

Body weight and lung weight were evaluated over the duration of the study to determine the effects of bleomycin and LYT-100 in the model. Body weight gain was impeded in Groups 2 and 3 that received Bleomycin between Days 1 to 9 (FIG. 31). With continued reference to FIG. 31, from Day 10 and until the end of Phase 1 on Day 14, body weight gain in Groups 2 and 3 resumed at a rate similar to Group 1 that received saline. Body weight gain (expressed as % of body weight compared with Day Minus 1 body weights) weight gain was impeded in Groups 2 and 3 that received Bleomycin between Days 1 to 9. From Day 10 and until the end of Phase 1 on Day 14, body weight gain in Groups 2 and 3 resumed at a rate similar to Group 1 that received saline.

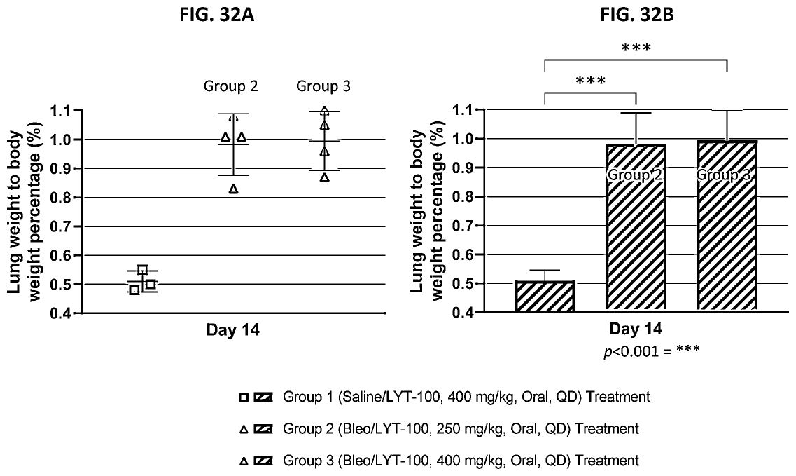

Lung weights were heavier in the bleomycin-treated animals (Group 1 vs Group 2 and Group 3 comparisons) as expected from this model. Lung weight ratios (expressed as % of body weight; FIGS. 32A and 32B) were heavier in the bleomycin-treated animals (Group 1 vs Group 2 and Group 3 comparisons) as expected from this model.

Overall, Phase 1 was performed as per protocol and no deviations were considered to affect the integrity of the Phase's outcome. During Phase 1 (Tolerability), LYT-100 was administered at high (400 mg/kg) and low (250 mg/kg) dose levels once daily (QD) from Day 8 until (including) Day 13 in healthy (high dose) and bleomycin-challenged (low and high dose) rats. LYT-100 was well tolerated by all animals and there was not an obvious correlation between dose level and presence of side effects. Any side effects observed were resolved within ˜5 hours after they were noticed and they did not reappear before the following dosing occasions. Based on the animals' body weight developments, clinical signs, lung weights and lung weight to body weight ratios, the tolerability phase determined that LYT-100 administered QD at 400 mg/kg was well-tolerated by both healthy and bleomycin-challenged rats and that this dose levels will be used to examine LYT-100's therapeutic potential during Phase 2 (Efficacy).

### Phase II Study

Subsequently, a Phase II study was conducted to evaluate the efficacy of LYT-100 in the rat BLM induced lung fibrosis model. The Phase II study design is provided in Table 32.

TABLE 32
BLM Study Design- Phase II

| | | | Test Article | | Day 28 |
|---|---|---|---|---|---|
| | Intervention | Test Article | Dosing | Number of | Necropsy |
| Group | (days 1-7) | and Dose | (Day 8-27) | Animals | and analysis |
| 4 | Saline; Days | Vehicle | PO; oral gavage | N = 10 | Preterminal blood |
| | 1, 2, 3, 6, | control | as solution in | | for plasma |
| | and 7 | | 1% aq. CMC; | | Lungs removed |
| 5 | Bleomycin | Vehicle | QD | N = 12 | and weighed |
| | 0.45 mg/kg; | control | | | Left Lung Lobe |
| 6 | Days 1, 2, 3, | LYT-100 | | N = 12 | snap frozen for |
| | 6, and 7 | (400 mg/kg) | | | HP |
| 7 | | Nintedanib | PO; oral gavage | N = 10 | Right lung lobe |
| | | 60 mg/kg | as solution in | | inflation fixed |
| | | | 1% aq. CMC; | | |
| | | | BID | | |

For Groups 4, 5, 6, and 7, bleomycin and vehicle dosing were conducted as indicated in Table 32 (0.45 mg/kg, at 1696 IU/mg of Bleomycin or saline on Day 1, 2, 3, 6 and 7). On days 8 to 27, LYT-100 was dosed via oral gavage once daily, and nintedanib was dosed twice daily via oral gavage.

### Observations

Animals were observed for a variety of clinical signs and symptoms following bleomycin, saline, and LYT-100 dosing. All animals dosed with bleomycin or saline had 100% incidence of abnormal sounds on Days 1, 2, 3, 6 and 7 which was alleviated by the next study day, confirming dosing to the lung. All animals dosed with bleomycin (Groups 5 to 7) were observed with respiratory signs from Day 2, with 100% incidence of increased respiratory rate from Day 4 and until the end of the Study on Day 28. There was no observed increased respiratory rate for Group 4 that received saline. Respiratory signs are an indication of acute inflammation secondary to bleomycin challenge.

### Results

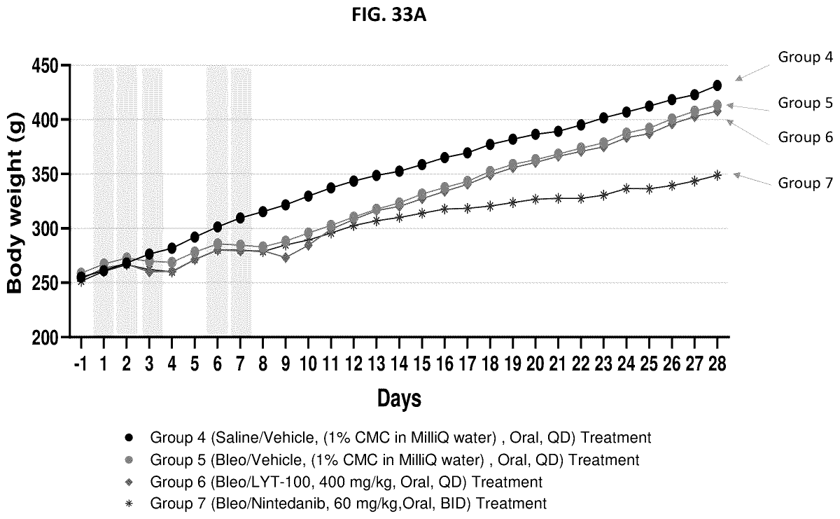

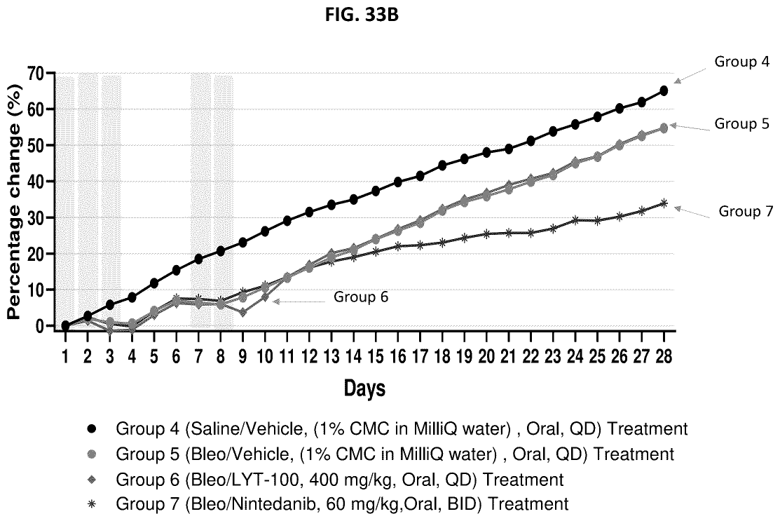

Body weight and lung weight were evaluated over the duration of the study to determine the effects of bleomycin and LYT-100 in the model. Body weight gain was impeded between Days 1 to 9 in Groups 5, 6, and 7 that received Bleomycin (FIG. 33A). With continued reference to FIG. 33A, from Day 10 and until the end of the efficacy Phase on Day 28, body weight gain in Groups 5 (Bleomycin/Vehicle) and 6 (Bleomycin/LYT-100) resumed and at a rate similar to Group 4 that received Saline/Vehicle. Body weight gain in Group 7 (Blemoycin/Nintedanib) showed modest improvement after Day 8 and the rate of body weight gain remained slower compared with the other groups. Body weight gain (expressed as % of body weight compared with Day 1 body weights) was impeded between Days 1 to 9 in Groups 5, 6, and 7 that received bleomycin (FIG. 33B). With continued reference to FIG. 33B, from Day 10 and until the end of the Efficacy Phase on Day 28, % of body weight gain in Groups 5 (Bleomycin/Vehicle) and 6 (Bleomycin/LYT-100) resumed and at a rate similar to Group 4 that received Saline/Vehicle. Percent of body weight gain in Group 7 (Bleomycin/Nintedanib) showed modest improvement after Day 8 and the rate of body weight gain remained slower compared with the other groups.

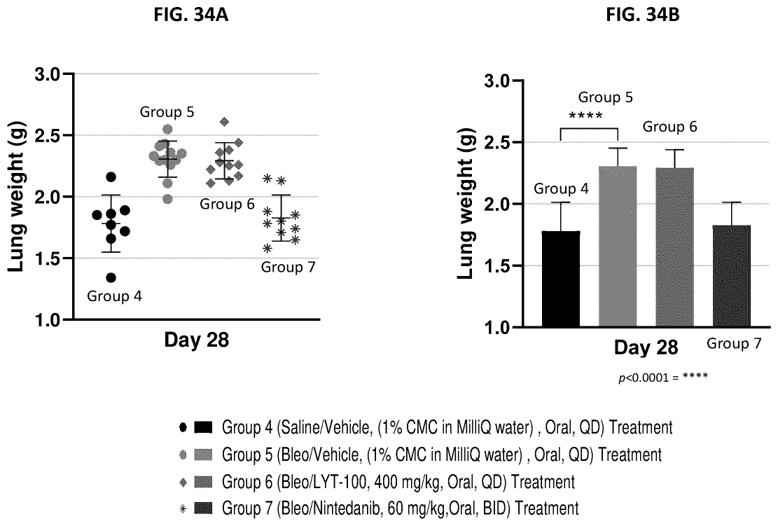

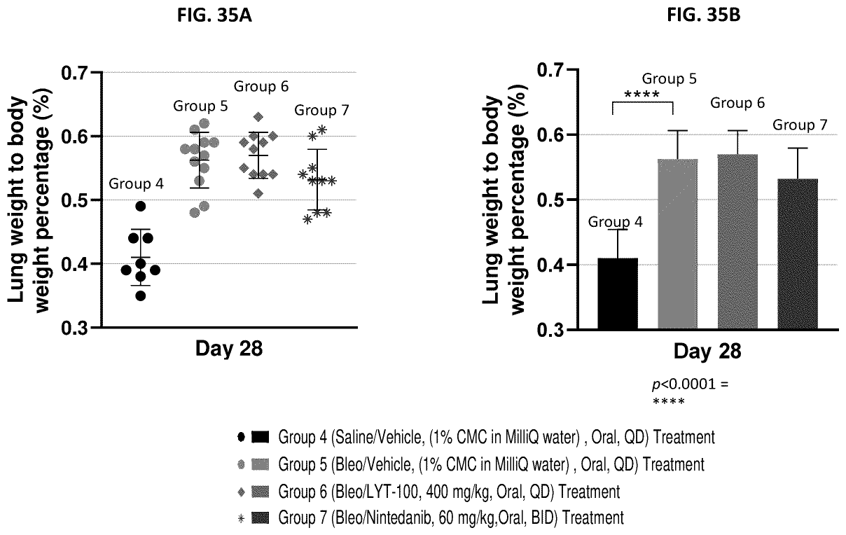

Mean lung weight increased in the bleomycin-treated rats (Group 4, saline vs Group 5, Bleomycin; FIGS. 34A and 34B). With continued reference to FIGS. 34A and 34B, LYT-100 treatment did not affect mean lung weight in the bleomycin-treated rats (Group 5, Bleomycin/vehicle vs Group 6, Bleomycin LYT-100). Nintedanib-treated rats had reduced lung weight (Group 7 vs Group 5) similar to non-challenged rats (Group 7 vs Group 4). Lung weight ratios (expressed as % percentage of body weight; FIGS. 35A and 35B) increased in the bleomycin-treated rats (Group 4, saline vs Group 5, Bleomycin). LYT-100 treatment did not affect lung weight ratios in the bleomycin-treated rats (Group 5, Bleomycin/vehicle vs Group 6, Bleomycin/LYT-100). There was a trend for lower lung weight ratios in the Nintedanib-treated rats (Group 5 vs Group 7), however this lung ratio remained higher compared with non-challenged rats (Group 7 vs Group 4).

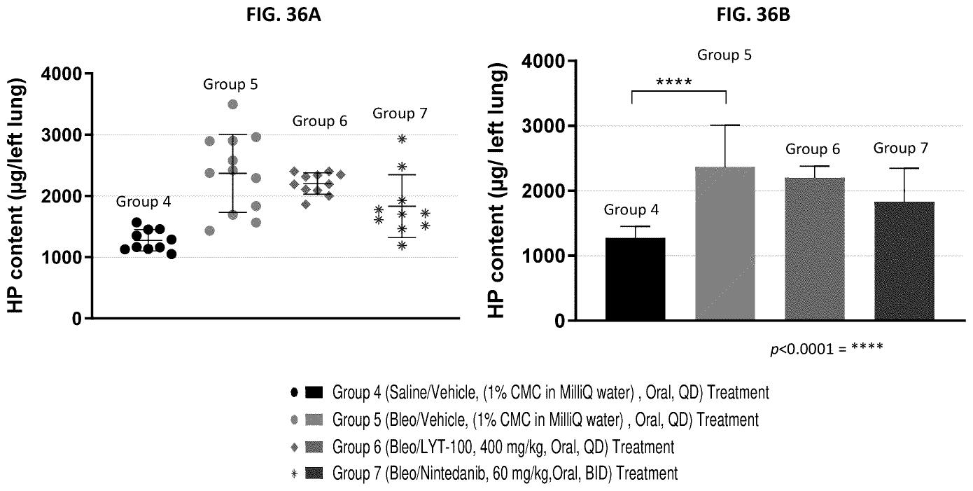

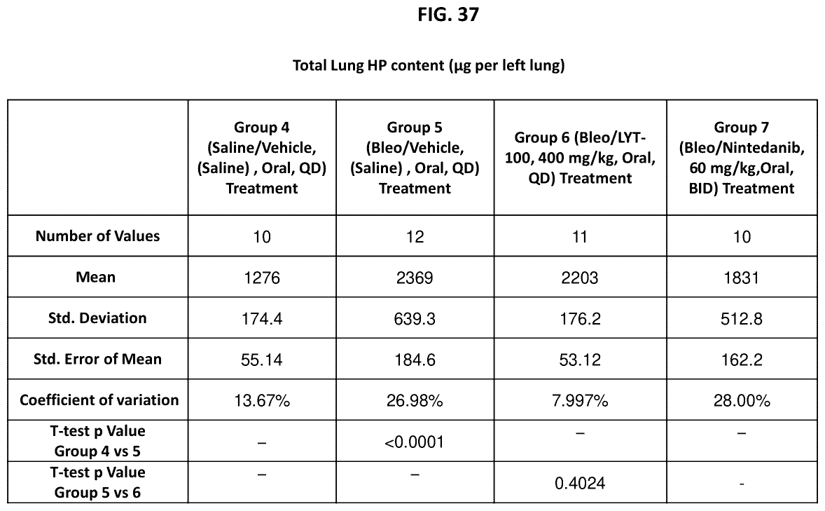

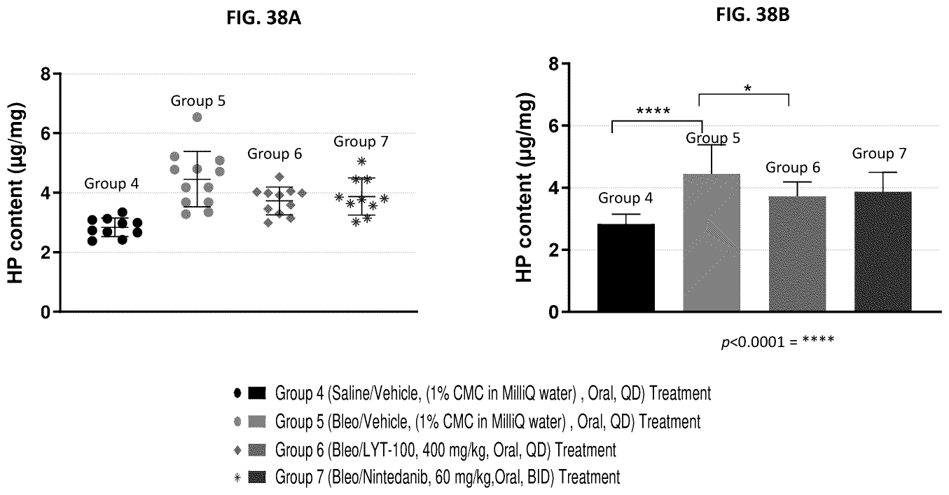

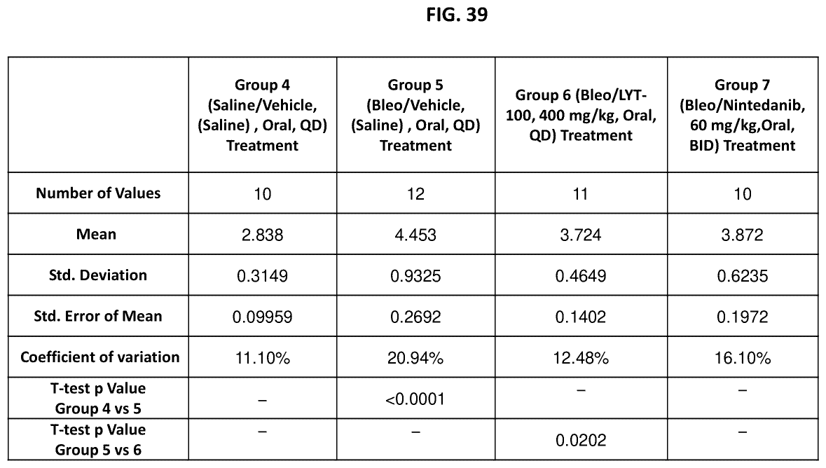

Lung hydroxyproline content was measured for all groups (FIGS. 36A, 36B, 37, 38A, 38B, 39). With reference to FIGS. 36A, 36B, 37, 38A, 38B, and 39, total left lung hydroxyproline (g per left lung) was higher in the bleomycin-treated rats (Group 4, saline vs Group 5, Bleomycin). LYT-100 treatment did not affect total hydroxyproline levels in the bleomycin-treated rats (Group 5, Bleomycin/vehicle vs Group 6, Bleomycin/LYT-100). Lungs from animals treated with Nintedanib had lower levels of total hydroxyproline (Group 7 vs Group 5) but higher than non-challenged rats (Group 7 vs Group 4). Hydroxyproline content (g per mg of wet lung) was higher in the bleomycin-treated rats (Group 4, saline vs Group 5, Bleomycin). LYT-100 treatment reduced the hydroxyproline content in the bleomycin-treated rats (Group 5, Bleomycin/vehicle vs Group 6, Bleomycin/LYT-100). Nintedanib treatment also reduced hydroxyproline content (Group 7 vs Group 5).

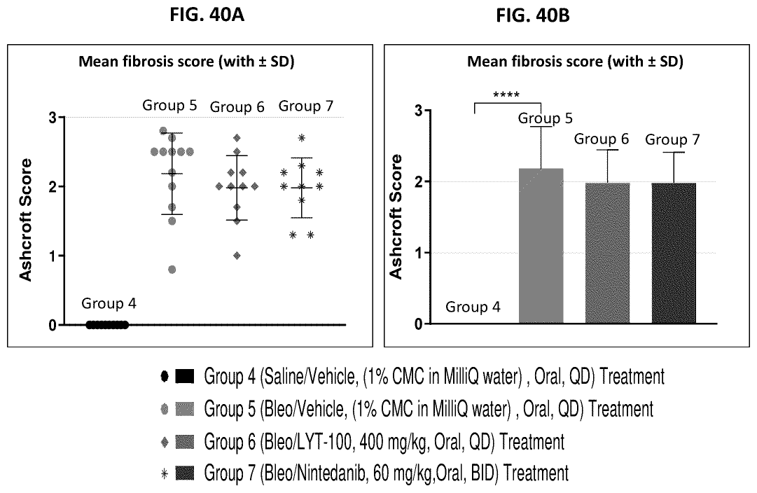

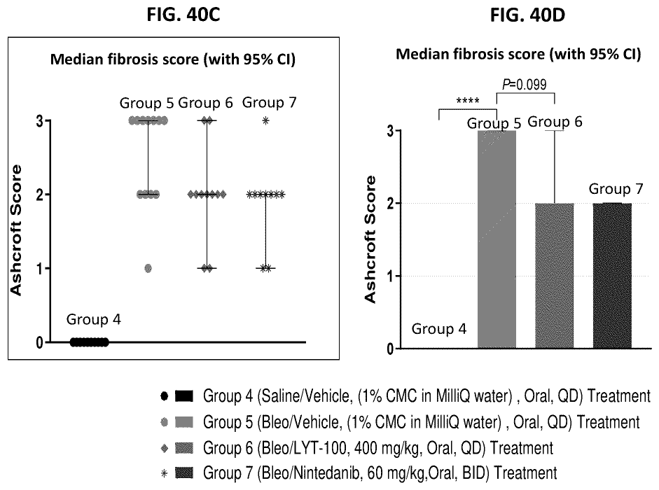

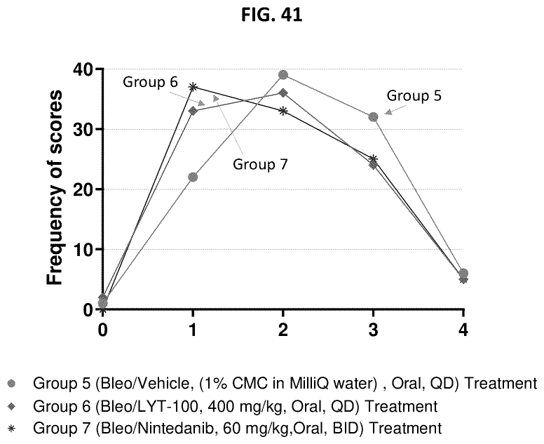

Histopathology studies were performed to evaluate the extent of fibrosis in lung (FIGS. 40A-40D and FIG. 41). Mean and median fibrosis scores increased in the Bleomycin-treated rats (Group 4, saline vs Group 5, Bleomycin). LYT-100 or nintedanib treatment did not affect the fibrosis scores (Group 5, Bleomycin/vehicle vs Group 6, Bleomycin/LYT-100 or Group 7, Bleomycin/Nintedanib). LYT-100 and nintedanib treatments reduced median fibrosis scores (Groups 6 and 7 compared with Group 5). The majority of the fibrosis scores in Group 5 (Bleomycin/vehicle) distributed around Score 2 (39% of the lung sections and 3 (32% of the lung sections). In the LYT-100 and nintedanib treatments (Groups 6 and 7, respectively) the distribution of lung section fibrosis scores shifted towards Scores 1 (33% and 37% respectively) and 2 (36% and 33% respectively).

Overall, Phase 2 was performed as per protocol and no deviations were considered to affect the integrity of the Phase's outcome. Mirroring Phase 1, LYT-100 administered QD at 400 mg/kg from Day 8 until (including) Day 27 was well tolerated by all animals and any side-effects observed were resolved within ˜5 hours after they were noticed and did not reappear before the following dosing occasions. Nintedanib administered twice daily (BID) at 60 mg/kg was used as a reference. LYT-100 did not negatively affect body weight developments, in contrast to nintedanib. LYT-100 reduced lung hydroxyproline content, suggesting reduced presence of connective tissue in the lungs. Consistent with the latter, lungs from LYT-100-treated rats also had reduced median fibrosis scores compared with vehicle controls.

### Example 12: Exploration of the Efficacy of LYT-100 in Treating Myocardial Fibrosis and Heart Failure

Patients with heart failure (HF) and evidence of myocardial fibrosis will be randomly assigned to receive LYT-100 or placebo for a period of time.

Inclusion criteria may include one or more of the following: HF with preserved ejection fraction (HFpEF), HF with reduced ejection fraction, HF with mid-range ejection fraction, elevated levels of natriuretic peptides, increased left ventricular end diastolic diameter, systolic dyssynchrony, and elevated filling pressures. The extent of myocardial fibrosis may be measured using using one or more of cardiovascular magnetic resonance, myocardial extracellular volume, and load-independent intrinsic left ventricular myocardial stiffness.

Endpoints for evaluation may include one or more of the following: reduction in myocardial extracellular volume (ECV); increase in 6 minute walk test (6MWT); improved KCCQ score (0-100); improved KCCQ clinical summary score (0-100); improved KCCQ total symptom score (0-100); improved Left ventricular EDVi, ml m⁻²; improved Left ventricular ESVi, ml m⁻²; improved Left ventricular EF, %; improved Left ventricular mass index, g m⁻²; improved Native T1, ms; improved absolute myocardial ECM volume, ml; improved absolute myocardial cell volume, ml; improved E/A ratio; improved Lateral e′, cm s⁻¹; Septal e′, cm s⁻¹, improved Average E/e′, cm s⁻¹; improved GLS, %; improved PCr:ATP ratio (BCPSC); improved Right ventricular EDVi, ml m⁻²; improved Right ventricular EF (%); improved Right ventricular PAP, mm Hg; improved Left atrium volume, ml; improved Left atrium volume index, ml m⁻²; improved Left atrium strain (reservoir), %; improved Left atrium strain (booster), %; improved Left atrium strain (conduit), %.

1. A method of treating a fibrotic-mediated pulmonary disease or disorder, comprising administering to a subject in need thereof total daily dose from about 825 to about 2475 mg of a deuterium-enriched pirfenidone having the structure: 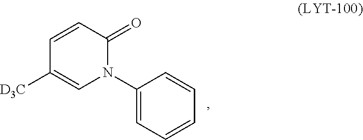 [SMILES: [2H]C([2H])([2H])c1ccc(=O)n(-c2ccccc2)c1]
wherein the fibrotic-mediated pulmonary disease or disorder is treated in the subject.

2. The method of claim 1, wherein the total daily dose is 1650 mg.

3. The method of claim 1, wherein the total daily dose is 2475 mg.

4. The method of any one of claims 1-3, wherein the total daily dose is administered in three equal administrations.

5. The method of claim 1, wherein the total daily dose is administered in three equal administrations of 825 mg each (825 mg TID).

6. The method of claim 1, wherein the total daily dose is administered in three equal administrations of 550 mg each (550 mg TID).

7. The method according to any one of claims 1-6, wherein the LYT-100 is administered without regard to food.

8. The method according to any one of claims 1-6, wherein the LYT-100 is administered without food.

9. The method according to any one of claims 1-6, wherein the LYT-100 is administered with food.

10. The method according to any one of claims 1-9, wherein the LYT-100 is administered without dose escalation.

11. The method according to any one of claims 1-9, wherein administering comprises titrating up to the total daily dose from an initial total daily dose which is below the total daily dose.

12. The method according to claim 5, wherein administering comprises titrating up to the total daily dose from an initial total daily dose which is below the total daily dose, and wherein titrating comprises administering the LYT-100 in three daily doses of 550 mg each for three days, followed by administering the LYT-100 in three daily doses of 825 mg each.

13. The method according to any one of claims 1-12, wherein the fibrotic-mediated pulmonary disease or disorder is an interstitial lung disease (ILD).

14. The method according to claim 14, wherein the ILD is an exposure-related ILD, a drug-induced ILD, an autoimmune interstitial lung disease, unclassifiable interstitial lung disease (uILD), progressive fibrotic interstitial lung disease (pfILD), respiratory bronchiolitis-ILD (RB-ILD), a connective tissue disease-related ILD (CTD-ILD), rheumatoid arthritis (RA-ILD), systemic sclerosis (SSc-ILD), mixed connective tissue disease-ILD, scleroderma related ILD, or ILD related to chronic sarcoidosis.

15. The method according to claim 14 or 15, wherein the ILD is a progressive fibrosing ILD (PF-ILD).

16. The method according to any one of claims 1-15, wherein the fibrotic- or collagen-mediated disease or disorder is alleviated.

17. The method according to any one of claims 1-16, wherein progression of the fibrotic- or collagen-mediated or disorder is delayed, slowed, or arrested.
